(19) United States
(12) Patent Application Publication
(10) Pub. No.: US 2024/0299510 A1
(43) Pub. Date: Sep. 12, 2024

(54) ANTIBODY-GUIDED PCSK9-MIMICKING IMMUNOGENS LACKING 9-RESIDUE SEQUENCE OVERLAP WITH HUMAN PROTEINS

(71) Applicant: The United States of America,as represented by the Secretary,Department of Health and Human Services, Bethesda, MD (US)

(72) Inventors: Peter D. Kwong, Washington, DC (US); Gwo-Yu Chuang, Rockville, MD (US); Baoshan Zhang, Bethesda, MD (US); Wing-Pui Kong, Germantown, MD (US); Yiran Wang, Nibley, UT (US); Cara Chao, Gaithersburg, MD (US); John R. Mascola, Rockville, MD (US); Li Ou, Potomac, MD (US); Tongqing Zhou, Boyds, MD (US); Yongping Yang, Potomac, MD (US); Bohan Liu, New York, NY (US); Cheng Cheng, Bethesda, MD (US); Daniel W. Biner, Rockville, MD (US); Reda Rawi, North Bethesda, MD (US)

(73) Assignee: The United States of America,as represented by the Secretary,Department of Health and Human Services, Bethesda, MD (US)

(21) Appl. No.: 18/270,436

(22) PCT Filed: Dec. 31, 2021
(86) PCT No.: PCT/US2021/065856

§ 371 Date: Jun. 29, 2023

Related U.S. Application Data
(60) Provisional application No. 63/132,640, filed on Dec. 31, 2020.

(51) Int. Cl. A61K 39/00; A61K 45/06; A61P 3/06; C12N 9/64

(52) U.S. Cl. CPC A61K 39/0005; A61K 45/06; A61P 3/06; C12N 9/6454; C12Y 304/21061

(57) ABSTRACT

The present disclosure describes the grafting of epitope residues from human PCSK9 to non-human PCSK9 or PCSK9 structural homologs and elimination of 9-mers oligopeptides that are found in human protein to reduce self-targeting response. Anti-genicity of the epitope-scaffold constructs was demonstrated toward anti-human PCSK9 antibodies. similar to wild type human PCSK9. It was shown that disclosed PCSK9 immunogens could significantly reduce LDL and cholesterol levels in immunized mice.

### CROSS-REFERENCE TO RELATED APPLICATIONS

This patent application claims the benefit of U.S. Provisional Patent Application No. 63/132,640, filed Dec. 31, 2020, which is incorporated by reference.

### STATEMENT REGARDING FEDERALLY SPONSORED RESEARCH AND DEVELOPMENT

This invention was made with Government support under project numbers Z01AI005024-19 and ZICAI005114-10 by the National Institutes of Health, National Institute of Allergy and Infectious Diseases. The Government has certain rights in the invention.

### TECHNICAL FIELD

The present invention is related to the fields of molecular biology, immunology and medicine. The invention provides a composition comprising immunogens with residues from human proprotein convertase subtilisin-kexin type 9 (PCSK9). The invention also provides methods for producing the compositions of the invention. The compositions of the invention are useful in the production of vaccines for the prevention, treatment or alleviation of PCSK9-related disorders, cardiovascular diseases, and other diseases and conditions, including dyslipidemias.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 863,561Byte ASCII (Text) file named “758174_ST25.txt,” dated Dec. 31, 2021.

### BACKGROUND OF THE INVENTION

Proprotein convertase subtilisin-kexin type 9 (hereinafter “PCSK9”), is a proteinase K-like subtilase identified as the 9th member of the mammalian PCSK family (Seidah et al, 2003 PNAS 100:928-933). The gene for PCSK9 localizes to human chromosome 1p33-p34.3, and is expressed in cells capable of proliferation and differentiation including, for example, hepatocytes, kidney mesenchymal cells, intestinal ileum, and colon epithelia as well as embryonic brain telencephalon neurons.

Structurally, PCSK9 includes a 30 amino acid signal peptide, followed by a prodomain (amino acids 31-152), a catalytic domain (amino acids 153-425) and a CHRD (C-terminal cysteine/histidine-rich domain) (amino acids 426-692). The NCBI reference sequence for human PCSK9 is NP_777596.2 (SEQ ID NO: 108). PCSK9 is synthesized as an approximately 72 kDa precursor protein that undergoes autocatalytic cleavage in the endoplasmic reticulum. After cleavage, the approximately 14 kDa prodomain remains tightly associated with the active site, rendering the mature protein catalytically inactive. PCSK9 undergoes a variety of post-translational modifications before being secreted efficiently from the cell.

In cell-culture systems, overexpression of PCSK9 resulted in a decrease in LDL-receptor (LDLR) levels via a post-transcriptional mechanism (Park, et al, 2004 J. Biol. Chem. 279:50630-638). It has been shown that PCSK9 forms a direct protein-protein interaction with the EGF-A (epidermal growth factor-like repeat A) domain of the LDLR that results in targeting of the LDLR to lysosomes for degradation (Kwon et al, 2008 PNAS 105:1820-1825: Zhang, et al, 2007 J. Biol. Chem. 282:18602-18612). This interaction appears to be necessary for PCSK9-mediated LDLR degradation (Li et al, 2007 Biochem. J. 406:203-207; McNutt et al, 2007 J. Biol. Chem. 282:20799-20803). Studies using surface plasmon resonance demonstrated that direct binding of PCSK9 to the LDLR could be abolished with three different anti-PCSK9 antibodies, and these antibodies blocked the PCSK9-LDLR interaction by inhibiting the action of PCSK9 produced endogenously (Duff et al, 2009 Biochem. J. 419:577-584). A neutralizing anti-PCSK9 monoclonal antibody that binds an epitope on PCSK9 adjacent to the region required for LDLR interaction was shown to inhibit PCSK9 binding to the LDLR and attenuate PCSK9-mediated reduction in LDLR protein levels, thereby increasing LDL uptake. Additionally, a combination of this antibody with a statin increased LDLR levels in HepG2 cells more than either treatment alone (Chan et al, 2009 PNAS 106:9820-25). Subsequently, a fully human PCSK9 monoclonal antibody was shown to increase the recycling of LDL receptors and reduce LDL cholesterol levels (Roth et al, N Engl J Med 2012, 367: 1891-900). Unfortunately, treatment to reduce LDL cholesterol by the administration of anti-PCSK9 antibodies is expensive and requires frequent administration of the antibodies to produce therapeutic effects.

Accordingly, it would be of great therapeutic benefit to produce a vaccine that could safely induce antibodies that antagonize the activity of PCSK9 for the treatment of various therapeutic conditions associated with PCSK9, such as increased plasma levels of LDL cholesterol.

### BRIEF SUMMARY OF THE INVENTION

The present invention relates to immunogens produced by the grafting of epitope residues from human PCSK9 to non-human PCSK9 or PCSK9 structural homologs and elimination of all 9 residue sequence overlaps with human protein to reduce self-targeting response. In the context of the invention, the grafted “epitope” residues are residues that are part of the recognition site of an antibody that can reduce cholesterol by binding to an epitope on PCSK9. Also disclosed are vaccine constructs comprising such immunogens and an immunogenic carrier, as well as methods for producing such immunogens, and pharmaceutical compositions comprising such immunogens, as well as uses of such compositions, in the treatment, alleviation or prophylaxis of PCSK9-related disorders. Such pharmaceutical compositions optionally comprise one or several adjuvants. Such pharmaceutical compositions optionally comprise one or several pharmaceutical carriers.

In particular, the present disclosure relates to an antigenic PCSK9 peptide of the invention, or an immunogenic or pharmaceutical composition thereof, for use as a medicament preferably in treatment, alleviation or prophylaxis of diseases associated with elevated levels of cholesterol in a mammal.

The immunogens of the present disclosure are useful in the treatment of patients having, or at risk for, elevated LDL-cholesterol or a condition associated with elevated LDL-cholesterol, e.g., a lipid disorder (e.g., hyperlipidemia, type I, type II, type III, type IV, or type V hyperlipidemia, secondary hypertriglyceridemia, hypercholesterolemia, familial hypercholesterolemia, xanthomatosis, cholesterol acetyltransferase deficiency). The immunogens of the present disclosure are also useful in the treatment of patients having arteriosclerotic conditions (e.g., atherosclerosis), coronary artery disease, cardiovascular disease, and patients at risk for these disorders, e.g., due to the presence of one or more risk factors, such as hypertension, cigarette smoking, diabetes, obesity, or hyperhomocysteinemia. The immunogens of the present disclosure are also useful in the treatment of Alzheimer's disease in a patient.

In certain aspects, the immunogens of the present disclosure may be administered together with another agent, such as, for example a statin.

In one aspect, an immunogen of the present disclosure comprises grafting of human PCSK9 antibody epitopes to iridescent shark PCSK9 and removing all 9-residue sequence overlaps with human proteins.

In another aspect, an immunogen of the present disclosure comprises grafting of human PCSK9 antibody epitopes to iridescent shark PCSK9 and removing all 9-residue sequence overlaps with human proteins and further inserting at least one HRV-3C cleavage site to remove the C-terminal domain.

In another aspect, an immunogen of the present disclosure comprises grafting of human PCSK9 antibody epitopes to iridescent shark PCSK9 and removing all 9-residue sequence overlaps with human proteins and further inserting at least one HRV-3C cleavage site to remove the C-terminal domain and flanking the cleavage site with the peptide sequence gly-ser-gly.

In yet another aspect, an immunogen of the present disclosure comprises grafting of human PCSK9 antibody epitopes to iridescent shark PCSK9 and removing all 9-residue sequence overlaps with human proteins and adding a T-cell help peptide from tetanus toxoid to C-terminal.

In another aspect, an immunogen of the present disclosure comprises grafting of human PCSK9 antibody epitopes to iridescent shark PCSK9 and removing all 9-residue sequence overlaps with human proteins and introducing mutations in the peptide sequence to reduce non-specific cleavage.

In another aspect, an immunogen of the present disclosure comprises grafting of human PCSK9 antibody epitopes to iridescent shark PCSK9 and removing all 9-residue sequence overlaps with human proteins and modifying the catalytic site of PCSK9, and optionally truncating the N-terminal.

In another aspect, an immunogen of the present disclosure comprises grafting of human PCSK9 antibody epitopes to iridescent shark PCSK9 and removing all 9-residue sequence overlaps with human proteins and modifying the putative cleavage site in the loop region of PCSK9 by introducing mutations in the peptide sequence of PCSK9.

In yet another aspect, an immunogen of the present disclosure comprises grafting of human PCSK9 antibody epitopes to iridescent shark PCSK9 and removing all 9-residue sequence overlaps with human proteins and inactivating PCSK9 by mutation D186, and adding a Furin cleavage site to make sure PCSK9 cleaves correctly.

In yet another aspect, an immunogen of the present disclosure comprises grafting of human PCSK9 antibody epitopes to iridescent shark PCSK9 and removing all 8-residue sequence overlaps with human proteins.

In another aspect, an immunogen of the present disclosure comprises grafting of human PCSK9 antibody epitopes to iridescent shark PCSK9 and removing all 8-residue sequence overlaps with human proteins comprising identifying a single mutation to remove one such 8-residue sequence overlap.

In another aspect, an immunogen of the present disclosure comprises identifying an alternative PCSK9 homologous sequence and removing all 9-residue sequence overlaps with human proteins.

In another aspect, an immunogen of the present disclosure comprises identifying an alternative PCSK9 homologous sequence and removing all 8-residue sequence overlaps with human proteins.

In another aspect, an immunogen of the present disclosure comprises grafting of human PCSK9 antibody epitopes to iridescent shark PCSK9 and removing four of six identical 8-residue sequence overlaps with human proteins.

In yet another aspect, an immunogen of the present disclosure comprises grafting of human PCSK9 antibody epitopes to iridescent shark PCSK9 and modifying the calcium binding loop to stabilize PCSK9.

In a further aspect, an immunogen of the present disclosure comprises grafting of human PCSK9 antibody epitopes to iridescent shark PCSK9 and inserting an inhibitory peptide to reduce catalytic activity.

In another aspect, an immunogen of the present disclosure comprises grafting of human PCSK9 antibody epitopes to iridescent shark PCSK9 and attaching an immunogenic carrier rTTHc.

A related aspect is a composition comprising the immunogen of this aspect of the invention. Such composition may further comprise at least one adjuvant.

Another related aspect is a nucleic acid encoding the immunogen of this aspect of the invention, as well as an expression vector comprising such nucleic acid, and a host cell comprising such expression vector.

Another aspect of the invention is a method of preventing, alleviating, or treating a dyslipidemia in an individual, comprising administering a therapeutically effective amount of the immunogen to the individual. Dyslipidemia may include lipid disorders such as hyperlipidemia from type I, type II, type III, type IV, or type V hyperlipidemia, secondary hypertriglyceridemia, secondary hypercholesterolemia, familial hypercholesterolemia, familial combined hyperlipidemia, xanthomatosis, and lecithin:cholesterol acetyltransferase deficiency.

Another aspect of the invention is a method of preventing, alleviating, or treating a condition selected from atherosclerosis, coronary artery disease, cardiovascular disease, and Alzheimer's disease in an individual, comprising administering a therapeutically effective amount of the immunogen to the individual.

The following numbered paragraphs [0032]-[0115] contain statements of broad combinations of the inventive technical features herein disclosed:
1. A composition comprising a non-human proprotein convertase subtilisin-kexin type 9 (PCSK9) structural homolog which has been grafted with one or more human PCSK9 peptides, wherein the composition does not comprise any 9 or more sequential residue overlaps with human PCSK9.
2. The composition of aspect 1, wherein the PCSK9 structural homolog is selected from the group consisting of iridescent shark PCSK9, channel catfish PCSK9, black rockcod PCSK9, climbing perch PCSK9, pupfish PCSK9, a cold adapted subtilisin-like serine proteinase, keratinase from Meiothermus taiwanensis WR-220e, Proteinase K like enzyme from a psychrotroph Serratia, a cold adapted subtilisin-like serine proteinase, Cuticle-Degrading Protease from Paecilomyces lilacinus, and a serine protease from an extreme thermophile.
3. The composition of aspect 2, wherein the PCSK9 structural homolog is iridescent shark PCSK9.
4. The composition of aspect 3, comprising the peptide sequence of SEQ ID NO: 1.
5. The composition of aspect 3, comprising a peptide sequence having at least 95% identity to SEQ ID NO: 1.
6. The composition of aspect 3, comprising a peptide sequence having at least 90% identity to SEQ ID NO: 1.
7. The composition of any one of aspects 1 to 3, wherein a HRV-3C cleavage site is inserted to remove a portion of the C-terminal domain.
8. The composition of aspect 7, comprising a peptide sequence selected from the group consisting of SEQ ID NOs: 2 to 9.
9. The composition of any one of aspects 1 to 3 and 7, further comprising a T-cell helper peptide from tetanus toxoid.
10. The composition of aspect 9, comprising a peptide sequence selected from the group consisting of SEQ ID NOs: 10 to 12.
11. The composition of any one of aspects 1 to 3, 7 and 9, wherein the one or more human PCSK9 peptides comprises amino acid residue substitutions to reduce non-specific cleavage.
12. The composition of aspect 11, comprising a peptide sequence selected from the group consisting of SEQ ID NOs: 13 to 14.
13. The composition of any one of aspects 1 to 3, 7, 9 and 11, wherein a PCSK9 catalytic site is modified.
14. The composition of aspect 13, comprising a peptide sequence selected from the group consisting of SEQ ID NOs: 15 to 24.
15. The composition of any one of aspects 1 to 3, 7, 9, 11 and 13, wherein a putative cleavage site in the loop region of the PCSK9 structural homolog is mutated.
16. The composition of aspect 15, comprising a peptide sequence selected from the group consisting of SEQ ID NOs: 25 to 29.
17. The composition of any one of aspects 1 to 3, 7, 9, 11, 13 and 15, wherein the PCSK9 structural homolog is mutated at position D186 and a Furin cleavage site is added.
18. The composition of aspect 17, comprising a peptide sequence selected from the group consisting of SEQ ID NOs: 30 to 37.
19. The composition of any one of aspects 1 to 3, 7, 9, 11, 13, 15 and 17, wherein all 8 or more sequential residue overlaps with human PCSK9 are removed.
20. The composition of aspect 19, comprising a peptide sequence selected from the group consisting of SEQ ID NOs: 38 to 53.
21. The composition of any one of aspects 1 to 3, 7, 9, 11, 13, 15 and 17, wherein at least one 8 sequential residue overlaps with human PCSK9 is removed.
22. The composition of aspect 21, comprising a peptide sequence selected from the group consisting of SEQ ID NOs: 54-63 and 71 to 85.
23. The composition of any one of aspects 1 to 3, 7, 9, 11, 13, 15, 17, 19 and 21 wherein a Ca2+binding loop of PCSK9 is modified.
24. The composition of aspect 23, comprising a peptide sequence selected from the group consisting of SEQ ID NOs: 87 to 89.
25. The composition of any one of aspects 1 to 3, 7, 9, 11, 13, 15, 17, 19, 21 and 23, wherein an inhibitory peptide is inserted in the Ca2+binding loop.
26. The composition of aspect 25, comprising a peptide sequence selected from the group consisting of SEQ ID NOs: 90 to 91.
27. The composition of any one of aspects 1 to 3, 7, 9, 11, 13, 15, 17, 19, 21, 23 and 25, further comprising an immunogenic carrier.
28. The composition of aspect 27, wherein the immunogenic carrier is selected from the group consisting of rTTHc, a nanoparticle, SPYTAG, and SPYCATCHER.
29. The composition of aspect 28, wherein the immunogenic carrier is rTTHc.
30. The composition of aspect 29, comprising the peptide sequence selected from the group consisting of SEQ ID NOs: 92, 114, and 116.
31. The composition of aspect 29, comprising a peptide sequence having at least 95% identity to a sequence selected from the group consisting of SEQ ID NOs: 92, 114, and 116.
32. The composition of any one of aspects 1 to 3, 7, 9, 11, 13, 15, 17, 19, 21, 23, 25, and 27 to 29, wherein the immunogenic carrier is conjugated to a cysteine residue which has been added to the PCSK9 structural homolog.
33. The composition of aspect 32, comprising a peptide sequence selected from the group consisting of SEQ ID NOS: 108 to 112.
34. The composition of aspect 28, wherein the nanoparticle contains a SPYTAG.
35. The composition of aspect 34, wherein the peptide sequence is selected from the group consisting of SEQ ID NOS: 114 to 116, 119, 121, 122, 135 to 144, and 146.
36. The composition of aspect 28, wherein the nanoparticle contains a SPYCATCHER.
37. The composition of aspect 36, wherein the peptide sequence is selected from the group consisting of SEQ ID NOS: 117, 118, 120, 123-134, and 145.
38. The composition of aspect 3, comprising the peptide sequence of SEQ ID NO: 93.
39. The composition of any one of aspects 1, 2, 7, 9, 11, 13, 15, 17, 19, 21, 23, 25, 27 to 29, 32, 34 and 36, wherein the PCSK9 structural homolog is channel catfish PCSK9.
40. The composition of aspect 39, comprising a peptide sequence selected from the group consisting of SEQ ID NOs: 64, 65 and 94.
41. The composition of any one of aspects 1, 2, 7, 9, 11, 13, 15, 17, 19, 21, 23, 25, 27 to 29, 32, 34 and 36, wherein the PCSK9 structural homolog is black rockcod PCSK9.
42. The composition of aspect 41, comprising a peptide sequence selected from the group consisting of SEQ ID NOs: 66, 95 and 96.
43. The composition of any one of aspects 1, 2, 7, 9, 11, 13, 15, 17, 19, 21, 23, 25, 27 to 29, 32, 34 and 36, wherein the PCSK9 structural homolog is climbing perch PCSK9.
44. The composition of aspect 43, comprising a peptide sequence selected from the group consisting of SEQ ID NOs: 67, 97 and 98.
45. The composition of any one of aspects 1, 2, 7, 9, 11, 13, 15, 17, 19, 21, 23, 25, 27 to 29, 32, 34 and 36, wherein the PCSK9 structural homolog is pupfish PCSK9.
46. The composition of aspect 45, comprising a peptide sequence selected from the group consisting of SEQ ID NOs: 68 and 99.
47. The composition of any one of aspects 1, 2, 7, 9, 11, 13, 15, 17, 19, 21, 23, 25, 27 to 29, 32, 34 and 36, wherein the PCSK9 structural homolog is cold adapted subtilisin-like serine proteinase.
48. The composition of aspect 47, comprising a peptide sequence selected from the group consisting of SEQ ID NOs: 100 and 104.
49. The composition of any one of aspects 1, 2, 7, 9, 11, 13, 15, 17, 19, 21, 23, 25, 27 to 29, 32, 34 and 36, wherein the PCSK9 structural homolog is keratinase from Meiothermus taiwanensis WR-220e.
50. The composition of aspect 49, comprising a peptide sequence selected from the group consisting of SEQ ID NOs: 101 and 107.
51. The composition of any one of aspects 1, 2, 7, 9, 11, 13, 15, 17, 19, 21, 23, 25, 27 to 29, 32, 34 and 36, wherein the PCSK9 structural homolog is a Proteinase K like enzyme from a psychrotroph Serratia.
52. The composition of aspect 51, comprising a peptide sequence selected from the group consisting of SEQ ID NOs: 102 and 103.
53. The composition of any one of aspects 1, 2, 7, 9, 11, 13, 15, 17, 19, 21, 23, 25, 27 to 29, 32, 34 and 36, wherein the PCSK9 structural homolog is a Cuticle-Degrading Protease from Paecilomyces lilacinus.
54. The composition of aspect 53, comprising the peptide sequence of SEQ ID NO: 105.
55. The composition of any one of aspects 1, 2, 7, 9, 11, 13, 15, 17, 19, 21, 23, 25, 27 to 29, 32, 34 and 36, wherein the PCSK9 structural homolog is a serine protease from an extreme thermophile.
56. The composition of aspect 55, comprising the peptide sequence of SEQ ID NO: 106.
57. The composition of any one of aspects 1, 2, 7, 9, 11, 13, 15, 17, 19, 21, 23, 25, 27 to 29, 32, 34, 36, 39, 41, 43, 45, 47, 49, 51, 53, and 55 wherein the composition selectively binds to PCSK9-specific antibodies.
58. The composition of any one of aspects 1, 2, 7, 9, 11, 13, 15, 17, 19, 21, 23, 25, 27 to 29, 32, 34, 36, 39, 41, 43, 45, 47, 49, 51, 53, 55 and 57, wherein the composition further comprises at least one adjuvant.
59. The composition of any one of aspects 1 to 3, comprising a peptide sequence selected from the group consisting of SEQ ID NOs: 123 to 146.
60. A method of preventing, alleviating, or treating a dyslipidemia in an individual, comprising administering a therapeutically effective amount of the composition of any one of aspects 1 to 59 to the individual.
61. The method according to aspect 60, wherein the dyslipidemia is selected from a group of hyperlipidemias consisting of type I, type II, type III, type IV, or type V hyperlipidemia, secondary hypertriglyceridemia, secondary hypercholesterolemia, hypercholesterolemia, familial hypercholesterolemia, familial combined hyperlipidemia xanthomatosis, and lecithin:cholesterol acetyltransferase deficiency.
62. The method of aspect 60 or 61, wherein the composition is administered in combination with at least one additional therapeutic agent selected from the group consisting of a statin, a bile acid sequestrant, niacin, a fibric acid derivative, and a long chain alpha, omega-dicarboxylic acid.
63. A method of preventing, alleviating, or treating a condition selected from atherosclerosis, coronary artery disease, cardiovascular disease, acute coronary syndrome, and Alzheimer's disease in an individual, comprising administering a therapeutically effective amount of the composition of any one of aspects 1 to 59 to the individual.
64. The method of aspect 63, wherein the composition is administered in combination with at least one additional therapeutic agent selected from the group consisting of a statin, a bile acid sequestrant, niacin, a fibric acid derivative, and a long chain alpha, omega-dicarboxylic acid.
65. A method of preventing, alleviating, or treating hypertriglyceridemia in an individual, comprising administering a therapeutically effective amount of the composition of any one of aspects 1 to 59 to the individual.
66. The method of aspect 65, wherein the composition is administered to the individual in combination with at least one adjuvant.
67. The method of aspect 65 or 66, wherein the composition is administered to the individual in combination with at least one of a statin and a fibric acid derivative.
68. Use of the composition of any one of aspects 1 to 59 in the manufacture of a medicament for the treatment of a dyslipidemia selected from a group of hyperlipidemias consisting of type I, type II, type III, type IV, or type V hyperlipidemia, secondary hypertriglyceridemia, secondary hypercholesterolemia, hypercholesterolemia, familial hypercholesterolemia, familial combined hyperlipidemia xanthomatosis, and lecithin: cholesterol acetyltransferase deficiency.
69. Use of the composition of any one of aspects 1 to 59 in the manufacture of a medicament for the treatment of a disease state selected from atherosclerosis, coronary artery disease, cardiovascular disease, acute coronary syndrome, and Alzheimer's disease.
70. The composition of any one of aspects 1 to 59 for use in the treatment of a dyslipidemia selected from a group of hyperlipidemias consisting of type I, type II, type III, type IV, or type V hyperlipidemia, secondary hypertriglyceridemia, secondary hypercholesterolemia, hypercholesterolemia, familial hypercholesterolemia, familial combined hyperlipidemia xanthomatosis, and lecithin:cholesterol acetyltransferase deficiency.
71. The composition of any one of aspects 1 to 59 for use in the treatment of a disease state selected from atherosclerosis, coronary artery disease, cardiovascular disease, acute coronary syndrome, and Alzheimer's disease.
72. A nucleic acid encoding an antigenic proprotein convertase subtilisin-kexin type 9 (PCSK9) peptide and a non-human PCSK9 structural homolog wherein the nucleic acid does not encode any 9 or more sequential residue overlaps with human PCSK9.
73 The nucleic acid of aspect 72, wherein the nucleic acid is an RNA molecule.
74. The nucleic acid of aspect 72 or 73, wherein the nucleic acid encodes HIT01 (SEQ ID NO:1), HIT01-K21Q-R218E (SEQ ID NO:14), HIT01-Combo3 (SEQ ID NO:85), or HIT01-K21Q-R218E 9glycans-SpyT 3CHS (SEQ ID NO:146).
75. The nucleic acid of any one of aspects 72-74, operably linked to a promoter.
76. A vector comprising the nucleic acid of any one of aspects 71-75.
77. The vector of aspect 76, wherein the vector is a viral vector.
78. A host cell comprising the vector of aspect 76 or 77.
79. An immunogenic composition comprising the vector of aspect 76 or 77.
80. A method of preventing, alleviating, or treating a cancer in an individual, comprising administering a therapeutically effective amount of the composition of any one of aspects 1 to 59 to the individual.
81. The method of aspect 80, further comprising administering at least one cancer therapeutic agent to the individual.
82. The method of aspect 81, wherein the cancer therapeutic agent is an immune checkpoint therapy agent.

This Summary of the Invention is neither intended nor should it be construed as being representative of the full extent and scope of the present invention. Moreover, references made herein to “the present invention,” or aspects thereof, should be understood to mean certain aspects of the present invention and should not necessarily be construed as limiting all aspects to a particular description. The present invention is set forth in various levels of detail in the Summary of the Invention as well as in the attached drawings and the Description of Invention and no limitation as to the scope of the present invention is intended by either the inclusion or non-inclusion of elements, components, etc. in this Summary of the Invention. Additional aspects of the present invention will become more readily apparent from the Description of Invention, particularly when taken together with the drawings.

### BRIEF DESCRIPTION OF THE SEVERAL VIEWS OF THE DRAWINGS

FIGS. 1A-1D illustrate a particular design of a hPCKS9-mimic, referred to as HIT01, produced by grafting human PCSK9 antibody epitope residues on to Iridescent shark PCKS9, with no 9-residue sequence overlaps with human proteins. FIG. 1A illustrates the published crystal structures of human PCSK9 in complex with LDL-R and various therapeutic antibodies respectively. FIG. 1B shows the buried surface area of PCSK9 residues to LDL-R and various therapeutic antibodies respectively. FIG. 1C shows non-human PCSK9 with the least number of 9-residue sequences identical to 9-residue sequence found in human proteins. FIG. 1D illustrates schematic of design PCSK9-mimic HIT01 by grafting the hPCSK9 epitope residues on to iridescent shark PCKS9 and removing 9-residue sequence overlaps with human proteins.

FIGS. 2A-2C illustrate the antigenic and biophysical properties of HIT01. FIG. 2A illustrates the size-exclusion chromatography (SEC) profile of PCSK9-mimic HIT01. FIG. 2B illustrates the binding profile (left) of PCSK9-mimic HIT01 and (right) of human PCSK9 to various PCKS9-binding antibodies. FIG. 2C illustrates the apparent KD of HIT01 binding to AMG145 and J16 as determined by using Octet, solid lines are observed response curves and dotted lines are fitting curves.

FIGS. 3A-3D illustrate that immunization of HIT01 significantly reduces cholesterol and LDL-C in mice. FIG. 3A illustrates the immunization scheme. FIG. 3B illustrates the sera concentration of LDL-C, total cholesterol, and HDL-C at week 4, revealing that immunization with PCSK9 HIT01 showed significant reduction of LDL-C, HDL-C, and cholesterol compared to the PBS control group. FIG. 3C illustrates the sera Octet response against hPCSK9 and mPCSK9, with and without competition by antibody AMG145. FIG. 3D illustrates the correlation of sera response targeting AMG145 epitope, as defined by mPCSK9 binding Octet minus mPCSK9 binding Octet when competed by antibody AMG145 for cholesterol (left) and LDL-C (right).

FIG. 4 are photographs of SDS PAGE, which concern the stability of PCSK9 protein and PCSK9 HIT01. FIG. 4 illustrates SDS-PAGE gel image of recombinant PCSK9 wild type protein at reducing and non-reducing conditions (left panel): the image on right panel illustrates SDS-PAGE gel image of recombinant HIT01 protein at fresh, incubated 4° C. about 3-4 weeks. HIT01 proteins was cleaved into 5 fragments after incubation at 4° C. indicating HIT01 protein is not stable when stored at 4° C.

FIG. 5 depicts data concerning the Mass Spec of the PCSK9 HIT01 protein in solution (left) juxtaposed with a photograph of SDS-PAGE showing the five bands resolved from proteolysis of the PCSK9 HIT01 protein (right). Protein bands of cleaved HIT01 protein were cut off from SDS-PAGE gel and sent for Mass Spec analysis. The upper panel in FIG. 5 indicated the molecular weight of each band. Protein N-terminal sequences analysis were performed to identify the cleavage sites.

FIGS. 6A through 6D present data concerning the stability of HIT01-K21Q-R218E. FIG. 6A is SDS-PAGE data obtained at different protein storage buffer conditions. The results indicate the upper band shift between reducing and non-reducing condition is consistent at various buffers. FIG. 6B is the profile of HIT01-K21Q-R218E analyzed on a size exclusion chromatography column. FIG. 6C illustrates the size distribution of HIT01-K21Q-R218E protein analyzed on a dynamic light scattering instrument. FIG. 6D is SDS-PAGE data for fresh HIT01-K21Q-R218E and at one-week intervals for four successive weeks.

FIG. 7A through 7C present data from a mouse immunization study involving HIT01-K21Q-R218E. FIG. 7A illustrates the immunization scheme. FIG. 7B illustrates serum antibody responses of immunized animals when measured with each immunogen. FIG. 7C illustrates the sera concentration of LDL-C at week 4.

FIGS. 8A through 8M present data concerning nonhuman primate (NHP) immunization and antibody responses for HIT01-K21Q-R218E. FIG. 8A depicts the study protocol. FIG. 8B depicts data concerning longitudinal serum antibody levels for antibodies against human PCSK9 and HIT01-K21Q-R218E in each animal. FIG. 8C depicts data concerning the anti-HIT-01 or PCSK9 IgG response in response to HIT01-K21Q-R218E injection in NHP. FIG. 8D depicts data concerning the serum LDL levels pre- and post HIT01-K21Q-R218E injection in NHP. FIG. 8E depicts data concerning the serum cholesterol levels pre- and post HIT01-K21Q-R218E injection in NHP. FIG. 8F depicts data concerning the serum HDL levels pre- and post HIT01-K21Q-R218E injection in NHP. FIG. 8G presents pooled data for LDL, HDL, and cholesterol serum levels measured for animals in the NHP study at pre-bleed vs week 2 following the third immunization. FIG. 8H presents pooled data for LDL, HDL, and cholesterol serum levels measured for animals in the NHP study at pre-bleed vs week 2 following the fourth immunization. FIG. 8I presents pooled data for LDL, HDL, and cholesterol serum levels measured for animals in the NHP study pre-bleed vs week 14 to 26 following the third immunization. FIG. 8J presents data comparing pre-bleed t0 vs week 2 following the final immunization. FIG. 8K presents data comparing the average levels pre-bleed week-27 to week-4 vs week 2 following the fourth immunization. FIG. 8L presents data comparing the LDL, HDL, and cholesterol serum levels measured at the third immunization vs week 2 following the third immunization. FIG. 8M presents data comparing the LDL, HDL, and cholesterol serum levels measured at the fourth immunization vs week 2 following the fourth immunization.

FIG. 9 presents the results of a competition assay using AMG145 demonstrating that immunization in the NHP study elicited antibody responses targeting LDL-R binding sites. The top two panels compare serum probed with HIT01-K21Q-R218E alone or with AMG145, and the bottom two panels compare serum probed with wild type PCSK9 alone or with AMG145.

FIGS. 10A and 10B graphically represent embodiments of the invention in which an PCSK9 immunogen of the invention is optionally conjugated to an immunogenic carrier. FIG. 10A represents an embodiment in which the immunogenic carrier comprises the tetanus toxoid heavy chain C fragment (rTTHC), and in which a linker is employed to conjugate the PCSK9 immunogen (indicated by an oval) to the carrier (indicated by a rounded rectangle). FIG. 10B represents an embodiment in which the immunogenic carrier comprises a protein nanoparticle, and in which SPYCATCHER/SPYTAGS are employed to conjugate multiple copies of the PCSK9 immunogen of the invention to enable multivalent display of the immunogen on self-assembling nanoparticles.

FIGS. 11A and 11B graphically illustrate maps of exemplary vectors. FIG. 11A represents HIT01_3CHS in pVRC8400, and FIG. 11B represents HIT01-K12Q-R218E also in pVRC8400.

FIGS. 12A through 12C present data pertaining to HIT01-Combo3. FIG. 12A presents images of SDS-PAGE analysis. FIG. 12B presents a sequence alignment comparing HIT01-Combo3 with HIT01, with an indication of normal processing and unintended cleavage of HIT01. For proposes of FIG. 12B, the sequence presented for HIT01-Combo3 lacks the C-terminal tag (gglvprgshhhhhhsawshpqfek (SEQ ID NO: 159)) present in SEQ ID NO:85. FIG. 12C depicts data concerning the SEC profile of HIT01-Combo3 (upper right) and binding curves to various antibodies (bottom).

FIGS. 13A through 13Q present data pertaining to the construction of HIT01 derivative nanoparticle conjugates. FIG. 13A graphically illustrates glycan designs (N-linked sequons) to enhance HIT01-K21Q-R218E nanoparticle solubility. FIG. 13B presents the results of SDS-PAGE gel analysis for purified HIT01-K21Q-R218E 9glycans-SpyT protein. FIG. 13C presents data concerning the elution of HIT01 9 Glycan+SpyT-Encapsulin Spy Catcher from an SEC column. FIG. 13D are electromicrographs reflecting negative staining of HIT01 9 Glycan+SpyT-Encapsulin Spy Catcher conjugates. FIGS. 13E through 13N present data concerning the antibody binding activity of the HIT01-K21Q-R218E 9glycans-encapsulin nanoparticles immunogen (FIGS. 13E through 13I) as compared with HIT01QE (FIGS. 13J through 13N) to various antibodies as stated in the figures. FIGS. 13O through 13Q present data from a mouse immunization study involving the HIT01-K21Q-R218E 9glycans-encapsulin nanoparticles immunogen. FIG. 13O illustrates the immunization scheme for an animal (mouse) study involving HIT01-K21Q-R218E 9glycans-encapsulin nanoparticles immunogens. FIG. 13P illustrates serum antibody responses of immunized animals when measured with each immunogen. FIG. 13Q illustrates the sera concentration of LDL-C at week 4.

### DETAILED DESCRIPTION OF THE INVENTION

The present disclosure is drawn to immunogens produced by the grafting of epitope residues from human PCSK9 to non-human PCSK9 or PCSK9 structural homologs and elimination of all 9 residue sequence overlaps with human protein to reduce self-targeting response, and methods of using the same that are effective to lower blood cholesterol levels in a mammal and treat dyslipidemias and related disease states in a mammal without the frequency of administration required by passive immunity strategies. In certain aspects, discontinuous human PCSK9 epitopes were preserved in the grafting to non-human PCSK9 or PCSK9 structural homologs. As noted above, in the context of the invention, the grafted “epitope” residues are residues that are part of the recognition site of an antibody that can reduce cholesterol by binding to an epitope in PCSK9.

As used herein, the term “grafted” refers to attaching one or more linear organic polymer to at least one second linear organic polymer. In certain aspects, a first linear organic polymer can be a polypeptide, for example a PCSK9 homolog, and the second linear organic monomer can be a peptide of human PCSK9. In certain aspects, the grafting can be accomplished by preparing a polynucleotide which codes for a PCSK9 homolog as well as peptides of human PCSK9 along different regions of the same polynucleotide.

As used herein, the term “polypeptide” refers to a linear organic polymer consisting of 50 or more amino-acid residues bonded together in a chain wherein the amino acid residues are bonded to adjacent residues by a peptide bond.

As used herein, the term “peptide” refers to a linear organic polymer consisting of less than 50 amino-acid residues bonded together in a chain wherein the amino acid residues are bonded to adjacent residues by a peptide bond.

As used herein, “sequential residue overlap” refers to a region of comparison between two separate chains of amino acids which share amino acid sequence identity.

As used herein, “protein” encompasses any polypeptide or peptide.

As used herein, “homolog” refers to proteins of similar structure. The similarity may be due to shared ancestry, but it not necessarily due to shared ancestry. Thus, homolog as used herein refers to similarity in structure, independent of any phylogenetic relationship between the organisms from which the structures derive or the in vivo biological function of the structures.

As used herein, “self-targeting response” is a specific adaptive immune response mounted against self-antigens.

As used herein, “T-cell helper peptide” is any peptide which can enhance T-cell response to an immunogenic composition.

### PCSK9 Immunogens of the Present Disclosure

In one aspect, the epitope residues from human PCSK9 were grafted to non-human PCSK9 or PCSK9 structural homologs and all 9-residue sequence overlaps with human protein were eliminated to reduce self-targeting response. It was found that some of such epitope-scaffold designs demonstrated antigenicity toward anti-human PCSK9 antibodies similar to wild-type human PCSK9. Such epitope scaffolds were conjugated to common carriers, nanoparticles or nanoparticle-carrier constructs to further enhance immunogenicity.

In one aspect, the PCSK9 immunogens comprise epitopes of PCSK9 comprising from 4 to 20 amino acids and, when administered to a subject, are able to lower the LDL-cholesterol level in blood of the subject. Preferably, the subject is a mammal, preferably a human. Preferably, the PCSK9 immunogen is able to lower the LDL-cholesterol level by at least 2%, 5%, 10%, 20%, 30%, 50%, or more. In one aspect, the PCSK9 immunogen is a portion of PCSK9, which participates in the interaction of PCSK9 with the LDL receptor. In one aspect, the PCSK9 immunogens is a portion of PCSK9 which has been mutated to change the peptide sequence, which participates in the interaction of PCSK9 with the LDL receptor.

In one aspect, an immunogen of the present disclosure comprises grafting of human PCSK9 antibody epitopes to iridescent shark PCSK9 and removing all 9-residue sequence overlaps with human proteins, e.g. SEQ ID NO: 1 (Table 1).

In another aspect, an immunogen of the present disclosure comprises grafting of human PCSK9 antibody epitopes to iridescent shark PCSK9 and removing all 9-residue sequence overlaps with human proteins and further inserting at least one HRV-3C cleavage site to remove the C-terminal domain, e.g. SEQ ID NOs: 2-5 (Table 2).

In another aspect, an immunogen of the present disclosure comprises grafting of human PCSK9 antibody epitopes to iridescent shark PCSK9 and removing all 9-residue sequence overlaps with human proteins and further inserting at least one HRV-3C cleavage site to remove the C-terminal domain and flanking the cleavage site with the peptide sequence gly-ser-gly, e.g. SEQ ID NOs: 6-9 (Table 2).

In yet another aspect, an immunogen of the present disclosure comprises grafting of human PCSK9 antibody epitopes to iridescent shark PCSK9 and removing all 9-residue sequence overlaps with human proteins and adding a T-cell help peptide from tetanus toxoid to C-terminal, e.g. SEQ ID NOs: 10-12 (Table 3).

In another aspect, an immunogen of the present disclosure comprises grafting of human PCSK9 antibody epitopes to iridescent shark PCSK9 and removing all 9-residue sequence overlaps with human proteins and introducing mutations in the peptide sequence to reduce non-specific cleavage, e.g. SEQ ID NOs: 13-14 (Table 4). These two nonlimiting examples are also identified as HIT01-K21Q-R218Q (SEQ ID NO:13) and HIT01-K21Q-R218E (SEQ ID NO:14).

In another aspect, an immunogen of the present disclosure comprises grafting of human PCSK9 antibody epitopes to iridescent shark PCSK9 and removing all 9-residue sequence overlaps with human proteins and modifying the catalytic site of PCSK9, and optionally truncating the N-terminal, e.g. SEQ ID NOs: 15-24 (Table 4).

In another aspect, an immunogen of the present disclosure comprises grafting of human PCSK9 antibody epitopes to iridescent shark PCSK9 and removing all 9-residue sequence overlaps with human proteins and modifying the putative cleavage site in the loop region of PCSK9 by introducing mutations in the peptide sequence of PCSK9, e.g. SEQ ID NOs: 25-29 (Table 4), and SEQ ID NO: 93 (Table 9).

In yet another aspect, an immunogen of the present disclosure comprises grafting of human PCSK9 antibody epitopes to iridescent shark PCSK9 and removing all 9-residue sequence overlaps with human proteins and inactivating PCSK9 by mutation D186, and adding a Furin cleavage site to make sure PCSK9 cleaves correctly, e.g. SEQ ID NOs: 30-37 (Table 4).

In yet another aspect, an immunogen of the present disclosure comprises grafting of human PCSK9 antibody epitopes to iridescent shark PCSK9 and removing all 8-residue sequence overlaps with human proteins, e.g. SEQ ID NOs: 38-53 (Table 5).

In another aspect, an immunogen of the present disclosure comprises grafting of human PCSK9 antibody epitopes to iridescent shark PCSK9 and removing all 8-residue sequence overlaps with human proteins comprising identifying a single mutation to remove one such 8-residue sequence overlap, e.g. SEQ ID NOs: 54-63, 73-84 (Table 5).

In another aspect, an immunogen of the present disclosure comprises identifying an alternative PCSK9 homologous sequence and removing all 9-residue sequence overlaps with human proteins, e.g. SEQ ID NO: 64 (Table 5).

In another aspect, an immunogen of the present disclosure comprises identifying an alternative PCSK9 homologous sequence and removing all 9-residue and 8-residue sequence overlaps with human proteins, e.g. SEQ ID NO: 65 (Table 5).

In another aspect, an immunogen of the present disclosure comprises identifying an alternative PCSK9 homologous sequence and removing some or all 8-residue sequence overlaps with human proteins, e.g. SEQ ID NOs: 66-70, 85-86 (Table 5).

In another aspect, an immunogen of the present disclosure comprises grafting of human PCSK9 antibody epitopes to iridescent shark PCSK9 and removing four of six identical 8-residue sequence overlaps with human proteins, e.g. SEQ ID NOs: 71-72 (Table 5).

In yet another aspect, an immunogen of the present disclosure comprises grafting of human PCSK9 antibody epitopes to iridescent shark PCSK9 and modifying the calcium binding loop to stabilize PCSK9, e.g. SEQ ID NOs: 87-89 (Table 6).

In a further aspect, an immunogen of the present disclosure comprises grafting of human PCSK9 antibody epitopes to iridescent shark PCSK9 and inserting an inhibitory peptide to reduce catalytic activity, e.g. SEQ ID NOs: 90-91 (Table 7).

In another aspect, an immunogen of the present disclosure comprises grafting of human PCSK9 antibody epitopes to iridescent shark PCSK9 and attaching an immunogenic carrier rTTHc, e.g. SEQ ID NOs: 92 (Table 8).

In another aspect, an immunogen of the present disclosure comprises grafting of human PCSK9 antibody epitopes to channel catfish PCSK9, blackrock cod PCSK9, climbing perch PCSK9, pupfish PCSK9, cold adapted subtilisin-like serine proteinase, keratinase from Meiothermus taiwanensis WR-220e, Proteinase K like enzyme from a psychrotroph Serratia, Cuticle-Degrading Protease from Paecilomyces lilacinus, serine protease from an extreme thermophile, Thermus aquaticus YT-1, or keratinase from Meiothermus taiwanensis WR-220e and removing all 9-residue sequence overlaps with human proteins, e.g. SEQ ID NOs: 94-107 (Table 9).

In another aspect, an immunogen of the present disclosure comprises grafting of human PCSK9 antibody epitopes to iridescent shark PCSK9 and removing all 9-residue sequence overlaps with human proteins, introducing mutations in the peptide sequence to facilitate N-linked glycosylation, and optionally introducing mutations in the peptide sequence to reduce non-specific cleavage, e.g. SEQ ID NOs: 123-146 (Table 11).

Such PCSK9 immunogens may be used alone or in combination, preferably when conjugated to an immunogenic carrier, to induce auto anti-PCSK9 antibodies in a subject in order to treat, prevent or ameliorate PCSK9-related disorders. The ability of the immunogenic PCSK9 immunogens of the invention to induce auto anti-PCSK9 antibodies may be measured in mice, using the test disclosed in Example 6 of the present application. The ability of auto-antibodies induced by the antigenic PCSK9 peptide of the invention to decrease the level of circulating plasma cholesterol may be measured in mice using the Roche Cobas 6000 c501 automated chemistry analyzer. The term “PCSK9 immunogen biological activity”, when used herein, refers to the ability of the antigenic PCSK9 peptides of the invention to induce auto anti-PCSK9 antibodies in a patient.

Vaccine constructs of the present disclosure, when administered to a subject, are able to lower the LDL-cholesterol level in blood of the subject. Preferably, the subject is a mammal, preferably a human. Preferably, the vaccine construct is able to lower the LDL-cholesterol level by at least 2%, 5%, 10%, 20%, 30%, 50%, or more.

In certain aspects, PCSK9 immunogens may be used alone or in combination to induce anti-PCSK9 antibodies in a subject in order to treat, prevent or ameliorate cancer. In certain aspects, the PCSK9 immunogens disclosed herein can be administered to a subject together with at least one cancer therapeutic agent. In certain aspects, the PCSK9 immunogens disclosed herein can be administered to a subject to potentiate immune checkpoint therapy.

PCSK9 immunogens can be further modified by amino acids, especially at the N- and C-terminal ends to allow the antigenic PCSK9 peptide to be conformationally-constrained and/or to allow coupling of the antigenic PCSK9 peptide to an immunogenic carrier after appropriate chemistry has been carried out.

The PCSK9 immunogens disclosed herein encompass functionally active variant peptides derived from the amino acid sequence of PCSK9 in which amino acids have been deleted, inserted or substituted without essentially detracting from the immunological properties thereof, i.e. such functionally active variant peptides retain a substantial antigenic PCSK9 peptide biological activity.

In one aspect, such functionally active variant peptides exhibit at least 60%, 65%, 70%, 75%, 80%, 85%, 90% or 95% identity to any one of SEQ ID NOs: 1-146.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA using default or recommended parameters. A program in GCG Version 6.1. FASTA (e.g., FASTA2 and FASTA3) provides alignments and percent sequence identity of the regions of the best overlap between the query and search sequences (Pearson, Methods Enzymol. 183:63-98 (1990): Pearson, Methods Mol. Biol. 132:185-219 (2000)). An alternative algorithm when comparing a sequence of the invention to a database containing a large number of sequences from different organisms is the computer program BLAST, especially blastp or tblastn, using default parameters. See, e.g., Altschul et al., J. Mol. Biol. 215:403-410 (1990): Altschul et al., Nucleic Acids Res. 25:3389-402 (1997).

Included in the scope of the aspects of the invention are functional variants of the inventive polypeptides or proteins described herein. The term “functional variant” as used herein refers to a polypeptide or protein having substantial or significant sequence identity or similarity to a parent polypeptide or protein, which functional variant retains the biological activity of the polypeptide or protein of which it is a variant. Functional variants encompass, for example, those variants of the polypeptide or protein described herein (the parent polypeptide or protein) that retain the ability to be recognized by PCSK9 antibodies to a similar extent, the same extent, or to a higher extent, as the parent polypeptide or protein. In reference to the parent polypeptide or protein, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the parent polypeptide or protein.

Functionally active variants comprise naturally occurring functionally active variants such as allelic variants and species variants and non-naturally occurring functionally active variants that can be produced by, for example, mutagenesis techniques or by direct synthesis.

A functional variant can, for example, comprise the amino acid sequence of the parent polypeptide or protein with at least one conservative amino acid substitution. Alternatively or additionally, the functional variants can comprise the amino acid sequence of the parent polypeptide or protein with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent polypeptide or protein.

Methods of introducing a mutation into amino acids of a protein are well known to those skilled in the art. See, e.g., Ausubel (ed.), Current Protocols in Molecular Biology, John Wiley and Sons, Inc. (2003): T. Maniatis, E. F. Fritsch and J. Sambrook, Molecular Cloning: A Laboratory Manual, Cold Spring Harbor laboratory, Cold Spring Harbor, N.Y. (2012).

Mutations can also be introduced using commercially available kits such as “QUIKCHANGE™ SITE-DIRECTED MUTAGENESIS KIT” (STRATAGENE™) or directly by peptide synthesis. The generation of a functionally active variant to an antigenic PCSK9 peptide by replacing an amino acid which does not significantly influence the function of the antigenic PCSK9 peptide can be accomplished by one skilled in the art.

Amino acid substitutions of the inventive polypeptides or proteins are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

Examples of groups of amino acids that have side chains with similar chemical properties include 1) aliphatic side chains: glycine, alanine, valine, leucine, and isoleucine: 2) aliphatic-hydroxyl side chains: serine and threonine: 3) amide-containing side chains: asparagine and glutamine: 4) aromatic side chains: phenylalanine, tyrosine, and tryptophan: 5) basic side chains: lysine, arginine, and histidine: 6) acidic side chains: aspartic acid and glutamic acid: and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

The polypeptide or protein can consist essentially of the specified amino acid sequence or sequences described herein, such that other components, e.g., other amino acids, do not materially change the biological activity of the polypeptide, protein, functional portion, or functional variant.

The polypeptides or proteins of aspects of the invention (including functional portions and functional variants) can be of any length, i.e., can comprise any number of amino acids, provided that the polypeptides or proteins (or functional portions or functional variants thereof) retain their biological activity, e.g., antigenicity toward human PCSK9 antibodies. For example, the polypeptide or protein can be about 50 to about 5,000 amino acids long, such as 50, 70, 75, 100, 125, 150, 175, 200, 300, 400, 500, 600, 700, 800, 900, 1,000, or more amino acids in length.

The polypeptides or proteins of aspects of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxy proline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′, N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α, γ-diaminobutyric acid, α,β-diaminopropionic acid, homophenylalanine, and α-tert-butylglycine.

The polypeptides or proteins of aspects of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized. Alternatively, in preferred embodiments, the polypeptides or proteins of aspects of the invention (including functional portions and functional variants) can be engineered to eliminate or reduce glycosylation, amidation, carboxylation, phosphorylation, esterification, N-acylatation, cyclization, etc. In one example, an embodiment of the invention includes a sequence, such as set forth herein, modified to remove N-linked glycosylation. For example, see SEQ ID Nos:96, 98, 99-101, 103-107; similar amino acid substitutions can be made to other embodiments of the polypeptides or proteins of aspects of the invention to remove sites for N-linked glycosylation.

The polypeptides or proteins of aspects of the invention (including functional portions and functional variants thereof) can be obtained by methods known in the art. The polypeptides or proteins may be made by any suitable method of making polypeptides or proteins. Also, polypeptides and proteins can be recombinantly produced using the nucleic acids described herein using standard recombinant methods. See, e.g., Ausubel et al., supra. Further, some of the polypeptides or proteins of the aspects of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the polypeptides or proteins described herein (including functional portions and functional variants thereof) can be synthesized by any of a variety of commercial entities. In this respect, the inventive polypeptides or proteins can be synthetic, recombinant, isolated, and/or purified.

PCSK9 immunogens of the present disclosure can also comprise those that arise as a result of the existence of multiple genes, alternative transcription events, alternative RNA splicing events, and alternative translational and postranslational events. A peptide can be expressed in systems, e.g. cultured cells, which result in substantially the same postranslational modifications present as when the peptide is expressed in a native cell, or in systems that result in the alteration or omission of postranslational modifications, e.g. glycosylation or cleavage, present when expressed in a native cell.

A PCSK9 immunogen of the present disclosure can be produced as a fusion protein that contains other non-PCSK9 or non-PCSK9-derived amino acid sequences, such as amino acid linkers or signal sequences or immunogenic carriers as defined herein, as well as ligands useful in protein purification, such as glutathione-S-transferase, histidine tag, and staphylococcal protein A. More than one PCSK9 immunogen of the disclosure can be present in a fusion protein. The heterologous polypeptide can be fused, for example, to the N-terminus or C-terminus of the peptide of the invention. A peptide of the present disclosure can also be produced as fusion proteins comprising homologous amino acid sequences, i.e., other PCSK9 or PCSK9-derived sequences.

The PCSK9 immunogen of the present disclosure might be linear or conformationally constrained. As used herein in reference to a peptide, the term “conformationally constrained” means a peptide, in which the three-dimensional structure is maintained substantially in one spatial arrangement over time. Conformationally constrained molecules can have improved properties such as increased affinity, metabolic stability, membrane permeability or solubility.

In addition, such conformationally constrained peptides are expected to present the antigenic PCSK9 epitope in a conformation similar to their native loop conformation, thereby inducing anti-PCSK9 antibodies more susceptible to recognize intact, native self PCSK9 molecules or with an increased affinity to recognize self PCSK9 molecules. Methods of conformational constraint are well known in the art and include, without limitation, bridging and cyclization.

Several approaches are known in the art to introduce conformational constraints into a linear peptide. For example, bridging between two neighboring amino acids in a peptide leads to a local conformational modification, the flexibility of which is limited in comparison with that of regular peptides.

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some aspects, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some aspects, the nucleic acid may encode additional amino acid sequences that do not affect the function of the polypeptide or protein and which may or may not be translated upon expression of the nucleic acid by a host cell. In an aspect of the invention, the nucleic acid is complementary DNA (cDNA). In an aspect of the invention, the nucleic acid comprises a codon-optimized nucleotide sequence.

The nucleic acids of an aspect of the invention may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can consist essentially of the specified nucleotide sequence or sequences described herein, such that other components, e.g., other nucleotides, do not materially change the biological activity of the encoded polypeptide, protein, PCSK9 immunogens, functional portion, or functional variant.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. The nucleic acids can be constructed based on chemical synthesis and/or enzymatic ligation reactions using procedures known in the art. See, for example, Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxy hydroxy methyl) uracil, 5-carboxy methylaminomethyl-2-thiouridine, 5-carboxy methylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxy carboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil. and 2.6-diaminopurine. Alternatively, one or more of the nucleic acids of the aspects of the invention can be purchased from any of a variety of commercial entities.

The nucleic acid can comprise any isolated or purified nucleotide sequence which encodes any of the polypeptides. proteins. PCSK9 immmunogens. conjugates. or functional portions or functional variants thereof disclosed herein. By way of non-limiting examples. a nucleic acid encoding HIT01 is

ATGCATCTGGTGCTGGTGCTGTGTCTGGCTGCCCTGGCCGCCTGTGATTACAGCG AGGACAAAGAAGTGAAGGCCCCTCAGCTGGATCACCCCGATCCTGGAACAGAGA GAGTGGCCGAGCTGCTGAGATGCACCAAGAGCGTTTGGAGAATCCCCGAGCAGT ACCTGGTGGTGCTGAGAGAGGGCACCAGAGATAGCCACGTGCAGAGAACCGTGT CTACCCTGAGAGCCCAGGCCGCTAGAAGAGGACACGCCATCCACATCATGCACA CCTACAGCGGCGTGTTCCACGGCTTTCTGATCAAGATGAGCAGCGAGGTGCTGCC CATGGCTCTGAAACTGCCTCACGTGGCCTACATCGAAGAGGACTCCTCTATCTTC GCCCAGAGCATCCCCTGGAACCTGCAGAGAATCATCCAGACCAAGCACGAGACA GGCAAGTACACCCCTCCTAACGATGGCGCCCAAGTGACCGTGTTCCTGCTGGATA CAAGCGTGCAGACCGACCACCGGGAAATCGAGGGCAAAGTGATGGTCACCGACT TCAACAGCATGCCCAAAGAGGACGGCACCCGGTTTCACAGATCTGCCAGCAAGT GTGAAAGCCACGGCACCCATATTGCCGGCGTGCTGTCTGGAAGAGATGCTGGCG TTGCAAGAGGCGTGTCCGTGAATACCGTGCGGGTGCTGAATTGCCAAGGCAGAG GAACAGTGTCAGGCGCTCTGGCCGGACTCGAGTATATCAGAGCCAGTCTGCAGG CCCAGCCTGTGTCTCCCGTGATCATCCTGCTGCCTTTCGTCGGCGGCTTCAGCAGA ACACTGAACACCGCCTGTCGCGAGATGGTGCATTCTGGCGCTGTGCTGATTGCCG CCGCTGGCAACTATCAGGACGACGCCTGTATGTACAGCCCCGCCTCTGAGCCTGA AGTGATCACAGTGGGAGCCAGCAACGCCGCCGATAGACCTCTCAGCTCTGGCAC CACAGGCACCAATCTGGGCAGATGCGTGGACGTTTTCGCCCCTGGCGAGGATATC ATCAGCGCCTCTGGCGATTGCAGCACCTGTTTCGTGTCTATGAGCGGCACCTCTC AGAGCGCTGCACATGCCGCTGGAATTGCTGCCGTCCTGCTGAACGCCTATCCTTC TGCTTCTCCCGCCGAAGTGCTGCAGCTGCTCAGATATCACGCTGTGCAGAGAGTG ATCAACCCCGACTCTCTGCCTCCTGAGCACTACCTGACCACACCTGATATGGTGG CCGCTCTGCCTACATCTGCCGCCACAGGCGAAAAGCTGCTGTGTAGAAGCGTGTG GTCCAAGAGAAGCGGCGTGGGCAGCTTTGATACAGCCGTGGCCAGATGCAGACA CGGCGAGGAAATGTTCAGCTGCTCCAGCTACAGCCCCAATGGCGTTCACGCCGG CGAGAGAATCGAGATCAGAGATGGCCAGAAAGTGTGCGAGGCCCACCACGGAA TTGGAGGACAAGGCGTGTACGCCGTGGCTAGGTGTTGTACAGGCAGCAGAGTGA AGTGTCACGCCTCTGCCTCACTGCATGTGGGCATTGATGCCGAGTGTCCCAGCCA AGAGTTTCAGCTGACCGGCTGCAGCAGCCACTACATCAGATCCCAGGATGTGGCT CAGCCCAGCTGGCCACTGCACAGCAATAGAAAAGCCTGTCCTGCCGGCGAAGGC GGAACAAGCCATGCCTTTTGTTGTCACGCCCCTAACCTGGAATGCCACCTGATCG AACACCACCAGAGCGAGTTCACCAAACAGGTGGAAGTGTCCTGCGAGGACAGCT GGACCCTGACAGGCTGTAATGCCGTGTCTCACGGCTCTGTGACCCATGCCGCTTA CACCAGAGGCAATACCTGCGTGATCCAGATGTTCGGCGGCGATAAGGGCGCTGC CGCCATTGCCATCTGCTGCAGATACAGACCCCTGGACCAGCAGTCCAACAACAA CCACGAGCAGAATACCGGCGGACTGGAAGTGCTGTTTCAAGGCCCTGGACACCA CCACCATCACCACTCTGCTTGGAGCCATCCTCAGTTCGAGAAGtgatga (SEQ ID NO:150), and a nucleic acid encoding HIT01-K21Q-R218E is ATGCATCTGGTGCTGGTGCTGTGTCTGGCTGCCCTGGCCGCCTGTGATTACAGCG AGGACCAGGAAGTGAAGGCCCCTCAGCTGGATCACCCCGATCCTGGAACAGAGA GAGTGGCCGAGCTGCTGAGATGCACCAAGAGCGTTTGGAGAATCCCCGAGCAGT ACCTGGTGGTGCTGAGAGAGGGCACCAGAGATAGCCACGTGCAGAGAACCGTGT CTACCCTGAGAGCCCAGGCCGCTAGAAGAGGACACGCCATCCACATCATGCACA CCTACAGCGGCGTGTTCCACGGCTTTCTGATCAAGATGAGCAGCGAGGTGCTGCC CATGGCTCTGAAACTGCCTCACGTGGCCTACATCGAAGAGGACTCCTCTATCTTC GCCCAGAGCATCCCCTGGAACCTGCAGAGAATCATCCAGACCAAGCACGAGACA GGCAAGTACACCCCTCCTAACGATGGCGCCCAAGTGACCGTGTTCCTGCTGGATA CAAGCGTGCAGACCGACCACCGGGAAATCGAGGGCAAAGTGATGGTCACCGACT TCAACAGCATGCCCAAAGAGGACGGCACCCGGTTTCACGAGTCTGCCAGCAAGT GTGAAAGCCACGGCACCCATATTGCCGGCGTGCTGTCTGGAAGAGATGCTGGCG TTGCAAGAGGCGTGTCCGTGAATACCGTGCGGGTGCTGAATTGCCAAGGCAGAG GAACAGTGTCAGGCGCTCTGGCCGGACTCGAGTATATCAGAGCCAGTCTGCAGG CCCAGCCTGTGTCTCCCGTGATCATCCTGCTGCCTTTCGTCGGCGGCTTCAGCAGA ACACTGAACACCGCCTGTCGCGAGATGGTGCATTCTGGCGCTGTGCTGATTGCCG CCGCTGGCAACTATCAGGACGACGCCTGTATGTACAGCCCCGCCTCTGAGCCTGA AGTGATCACAGTGGGAGCCAGCAACGCCGCCGATAGACCTCTCAGCTCTGGCAC CACAGGCACCAATCTGGGCAGATGCGTGGACGTTTTCGCCCCTGGCGAGGATATC ATCAGCGCCTCTGGCGATTGCAGCACCTGTTTCGTGTCTATGAGCGGCACCTCTC AGAGCGCTGCACATGCCGCTGGAATTGCTGCCGTCCTGCTGAACGCCTATCCTTC TGCTTCTCCCGCCGAAGTGCTGCAGCTGCTCAGATATCACGCTGTGCAGAGAGTG ATCAACCCCGACTCTCTGCCTCCTGAGCACTACCTGACCACACCTGATATGGTGG CCGCTCTGCCTACATCTGCCGCCACAGGCGAAAAGCTGCTGTGTAGAAGCGTGTG GTCCAAGAGAAGCGGCGTGGGCAGCTTTGATACAGCCGTGGCCAGATGCAGACA CGGCGAGGAAATGTTCAGCTGCTCCAGCTACAGCCCCAATGGCGTTCACGCCGG CGAGAGAATCGAGATCAGAGATGGCCAGAAAGTGTGCGAGGCCCACCACGGAA TTGGAGGACAAGGCGTGTACGCCGTGGCTAGGTGTTGTACAGGCAGCAGAGTGA AGTGTCACGCCTCTGCCTCACTGCATGTGGGCATTGATGCCGAGTGTCCCAGCCA AGAGTTTCAGCTGACCGGCTGCAGCAGCCACTACATCAGATCCCAGGATGTGGCT CAGCCCAGCTGGCCACTGCACAGCAATAGAAAAGCCTGTCCTGCCGGCGAAGGC GGAACAAGCCATGCCTTTTGTTGTCACGCCCCTAACCTGGAATGCCACCTGATCG AACACCACCAGAGCGAGTTCACCAAACAGGTGGAAGTGTCCTGCGAGGACAGCT GGACCCTGACAGGCTGTAATGCCGTGTCTCACGGCTCTGTGACCCATGCCGCTTA CACCAGAGGCAATACCTGCGTGATCCAGATGTTCGGCGGCGATAAGGGCGCTGC CGCCATTGCCATCTGCTGCAGATACAGACCCCTGGACCAGCAGTCCAACAACAA CCACGAGCAGAATACCGGCGGACTGGTGCCCAGGGGCAGCCACCACCACCATCA CCACTCTGCTTGGAGCCATCCTCAGTTCGAGAAGTGATGA (SEQ ID NO:151). Alternatively, the nucleotide sequence can comprise a nucleotide sequence which is degenerate to any of the sequences or a combination of degenerate sequences.

An aspect of the invention also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive polypeptides, proteins, PCSK9 immunogens, conjugates, or functional portions or functional variants thereof. It is generally appreciated that conditions can be rendered more stringent by the addition of increasing amounts of formamide.

The aspects of the invention also provide a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In an aspect, the nucleic acids of the aspects of the invention can be incorporated into a recombinant expression vector. In this regard, an aspect of the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the aspects of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

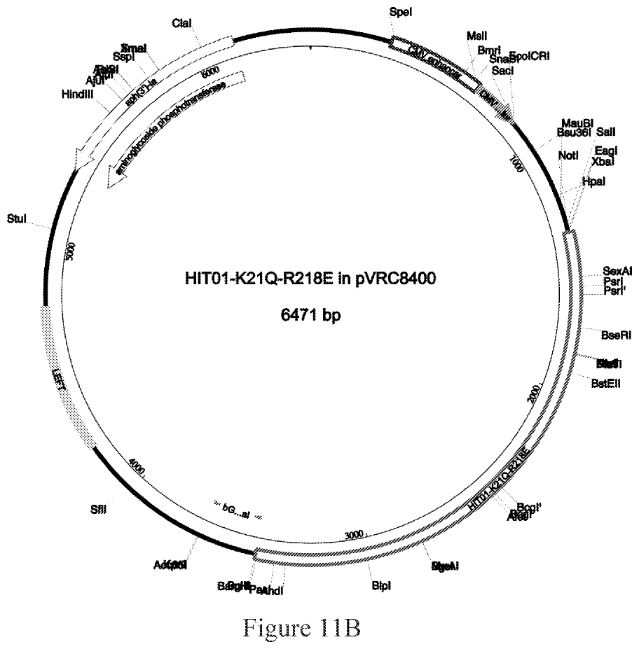

In an aspect, the recombinant expression vector can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (FERMENTAS LIFE SCIENCES, Glen Burnie, MD), the pBluescript series (STRATAGENE, LaJolla, CA), the pET series (NOVAGEN, Madison, WI), the pGEX series (PHARMACIA BIOTECH, Uppsala, Sweden), and the pEX series (CLONTECH, Palo Alto, CA). Nonlimiting examples of two vectors suitable for expressing HIT01 and HIT01-K21Q-R218E, respectively, based on pVRC8400, are diagrammed in FIGS. 11A and 11B, based on pVRC8400. Bacteriophage vectors, such as λGT10, λGT11, λZapII (STRATAGENE), λEMBL4, and λNM1149, also can be used. Examples of plant expression vectors include pBI01, pBI101.2, pBI101.3, pBI121 and pBIN19 (CLONTECH). Examples of animal expression vectors include pEUK-Cl, pMAM, and pMAMneo (CLONTECH). The recombinant expression vector may be a viral vector, e.g., a retroviral vector.

A number of transfection techniques are generally known in the art. Transfection methods include calcium phosphate co-precipitation, direct micro injection into cultured cells, electroporation, liposome mediated gene transfer, lipid mediated transduction, and nucleic acid delivery using high velocity microprojectiles.

In an aspect, the recombinant expression vectors can be prepared using standard recombinant DNA techniques described in, for example, Ausubel et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the polypeptides, proteins, PCSK9 immunogens, conjugates, or functional portions or functional variants thereof, or to the nucleotide sequence which is complementary to or which hybridizes to the nucleotide sequence encoding the inventive polypeptides, proteins, PCSK9 immunogens, conjugates, or functional portions or functional variants thereof. The selection of promoters, e.g., strong, weak, inducible, tissue-specific and developmental-specific, is within the ordinary skill of the artisan. Similarly, the combining of a nucleotide sequence with a promoter is also within the ordinary skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent.

An aspect of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant polypeptide, protein, PCSK9 immunogens, conjugate, or functional portion or functional variant thereof, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a B cell or a T cell.

Also provided by an aspect of the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one aspect of the invention, the population of cells is a clonal population comprising host cells comprising a recombinant expression vector as described herein.

### Immunogenic Carriers

In some aspects of the vaccine constructs of the present disclosure, an antigenic PCSK9 peptide of the invention is linked to an immunogenic carrier molecule to form immunogens for vaccination protocols, preferably wherein the carrier molecule is not related to the native PCSK9 molecule.

The term “immunogenic carrier” herein includes those materials which have the property of independently eliciting an immunogenic response in a host animal and which can be covalently coupled to a peptide, polypeptide or protein either directly via formation of peptide or ester bonds between free carboxyl, amino or hydroxyl groups in the peptide, polypeptide or protein and corresponding groups on the immunogenic carrier material, or alternatively by bonding through a conventional bifunctional linking group, or as a fusion protein.

In specific aspects, the immunogenic carrier may be a virus-like particle (VLP), preferably a recombinant virus-like particle.

As used herein, the term “virus-like particle” refers to a structure resembling a virus particle but which has been demonstrated to be nonpathogenic. In general, virus-like particles lack at least part of the viral genome. Also, virus-like particles can often be produced in large quantities by heterologous expression and can be easily purified. A virus-like particle in accordance with the invention may contain nucleic acid distinct from their genome. A typical and preferred aspect of a virus-like particle in accordance with the present invention is a viral capsid such as the viral capsid of the corresponding virus, bacteriophage, or RNA-phage.

In specific aspects, the immunogenic carrier used in combination with an PCSK9 immunogen of the invention is a Qbeta coat protein. Qbeta coat protein was found to self- assemble into capsids when expressed in E. coli (Kozlovska et al., 1993 Gene 137:133-137). The obtained capsids or virus-like particles showed an icosahedral phage-like capsid structure with a diameter of 25 nm and T=3 quasi symmetry. Further, the crystal structure of phage Qss has been solved. The capsid contains 180 copies of the coat protein, which are linked in covalent pentamers and hexamers by disulfide bridges (Golmohammadi et al., 1996 Structure 4: 5435554) leading to a remarkable stability of the capsid of Qbeta coat protein. Qbeta capsid protein also shows unusual resistance to organic solvents and denaturing agents. The high stability of the capsid of Qbeta coat protein is an advantageous feature, in particular, for its use in immunization and vaccination of mammals and humans in accordance of the present invention.

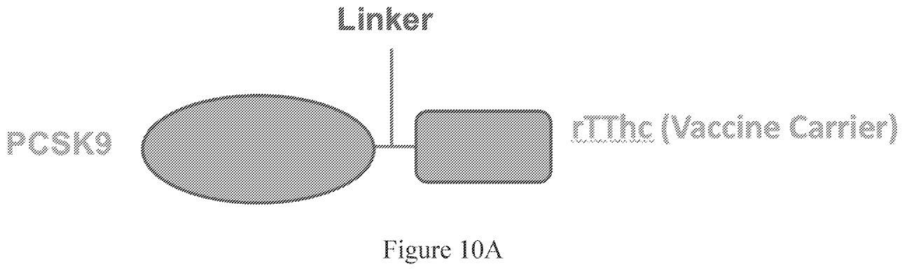

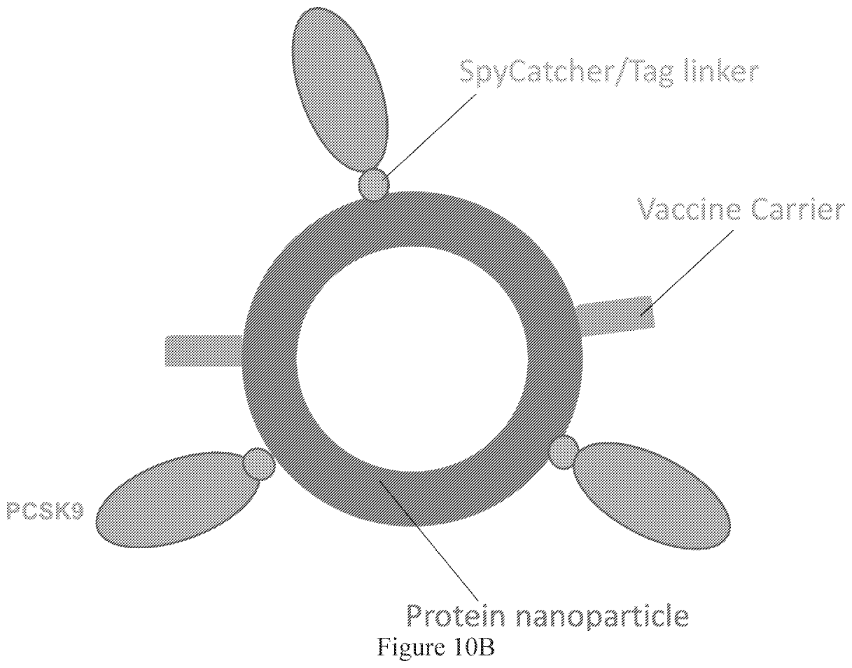

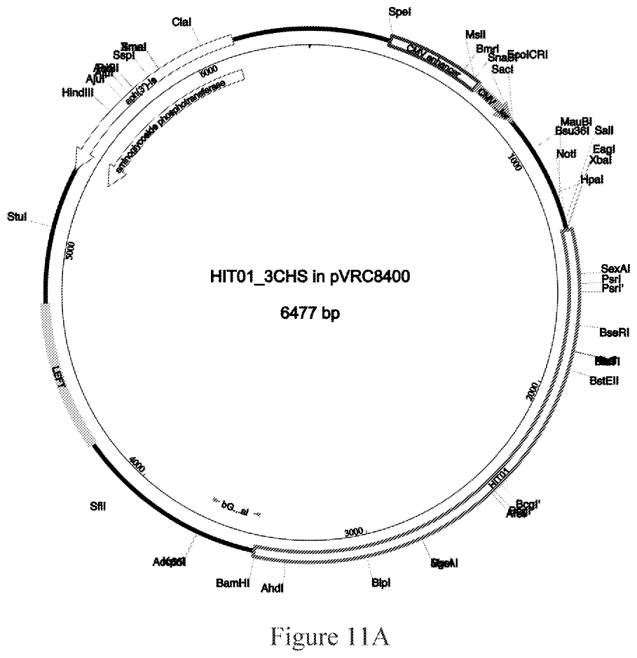

In some aspects, the immunogenic carrier is selected from keyhole limpet hemocyanin (KLH), tetanus toxoid heavy chain C fragment (rTTHC), H. influenzae protein D (HiD), cross-reactive material from diphtheria toxin (CRM197), and any combination thereof. In such an embodiment, an PCSK9 immunogen of the invention can be conjugated to the immunogenic carrier using a linker, such as is known in the art. Such a conformation is represented diagrammatically in FIG. 10A. Non-limiting examples of embodiments of the present invention incorporating rTTHC are set forth at SEQ ID NO:92 (HIT01 attached to immunogenic carrier rTTHc), SEQ ID NO: 114 (HIT01 fused to TTHC and SPYTAG), SEQ ID NO:116 (HIT01 fused to TTHC and SPYCATCHER), SEQ ID NO: 118 (HIT01 fused to TTHC and SPYCATCHER with five residue linker).

In some aspects, the immunogenic carrier can be or comprise a nanoparticle, SPYTAG, SPYCATCHER, and the like. Such a conformation is represented diagrammatically in FIG. 10B. “SPYTAG” and “SPYCATCHER” are components of a system for irreversible conjugation of recombinant proteins. The peptide SPYTAG (13 amino acids) spontaneously reacts with the protein SPYCATCHER (12.3 kDa) to form an intermolecular isopeptide bond between the pair. SPYTAG and SPYCATCHER were formed from the splitting and engineering of the CnaB2 domain of the FbaB protein from Streptococcus pyogenes. In such an embodiment, the use of SPYCATCHER/SPYTAG linkers enables multivalent display of the PCSK9 immunogen of the invention on self-assembling nanoparticles. Non-limiting examples of embodiments of the present invention incorporating SPYTAG and SPYCATCHER are set forth at SEQ ID NOs: 114-118 and 123-146. Non-limiting examples of self-assembling nanoparticles that can be employed as conjugates for multivalent display of the PCSK9 immunogen of the invention comprise for example, encapsulin (see, e.g., SEQ ID No: 119 (stabilized encapsulin fused with SPYTAG) and SEQ ID NO:120 (stabilized encapsulin fused with SPYCATCHER)), lumazine synthase (see, e.g., SEQ ID NO: 121 (lumazine synthase, with N71 mutation, fused with SPYTAG)), and ferritin (see, e.g., SEQ ID NO: 122 (ferritin, with N96 mutation, fused with SPYTTAG)), among others known to persons of ordinary skill.

Additionally, glycans can be used to enhance the solubility of the PCSK9 immunogen of the invention to facilitate multivalent display of the PCSK9 immunogen of the invention on such self-assembling nanoparticles. See, for example, Zhang et al. 2020 Sci Rep. 2020 Oct. 23:10(1): 18149. doi: 10.1038/s41598-020-74949-2.PMID: 33097791, demonstrating that the addition of an N-linked glycan to ferritin increases solubility of this nanoparticle. Thus, for example, the PCSK9 immunogen of the invention can comprise a molecule such as set forth in Sequence Table 11 (SEQ ID Nos: 123-146).

### Linker Constructs of the Invention

The antigenic PCSK9 peptides of the invention may be coupled to immunogenic carriers via chemical conjugation or by expression of genetically engineered fusion partners. The coupling does not necessarily need to be direct, but can occur through linker sequences (see, e.g., FIG. 10A). More generally, in the case that immunogens either fused, conjugated or otherwise attached to an immunogenic carrier, spacer or linker sequences are typically added at one or both ends of the immunogens. Such linker sequences generally comprise sequences recognized by the proteasome, proteases of the endosomes or other vesicular compartment of the cell.

In one aspect, the immunogens of the present disclosure are expressed as fusion proteins with the immunogenic carrier. Fusion of the immunogen can be effected by insertion into the immunogenic carrier primary sequence, or by fusion to either the N- or C-terminus of the immunogenic carrier. Hereinafter, when referring to fusion proteins of a peptide to an immunogenic carrier, the fusion to either ends of the subunit sequence or internal insertion of the peptide within the carrier sequence are encompassed. Fusion, as referred to hereinafter, may be effected by insertion of the antigenic peptide into the sequence of the carrier, by substitution of part of the sequence of the carrier with the antigenic peptide, or by a combination of deletion, substitution or insertions.

When the immunogenic carrier is a VLP, the chimeric immunogen-VLP subunit will be in general capable of self-assembly into a VLP. VLP displaying epitopes fused to their subunits are also herein referred to as chimeric VLPs. For example, EP 0 421 635 B describes the use of chimeric hepadnavirus core antigen particles to present foreign peptide sequences in a virus-like particle.

Flanking amino acid residues may be added to either end of the sequence of the antigenic peptide to be fused to either end of the sequence of the subunit of a VLP, or for internal insertion of such peptide sequence into the sequence of the subunit of a VLP. Glycine and serine residues are particularly favored amino acids to be used in the flanking sequences added to the peptide to be fused. Glycine residues confer additional flexibility, which may diminish the potentially destabilizing effect of fusing a foreign sequence into the sequence of a VLP subunit.

### Compositions Comprising an PCSK9 Immunogens

The present disclosure further relates to compositions, particularly immunogenic compositions comprising an antigenic PCSK9 immunogen linked to an immunogenic carrier, and optionally at least one adjuvant. Such immunogenic compositions are useful to prevent, treat or alleviate PCSK9-related disorders.

In some aspects, an immunogenic composition according to the invention comprises a PCSK9 immunogen, optionally comprising a linker, linked to an immunogenic carrier, selected from Qbeta, DT, PP7, PPV or Norwalk Virus VLP.

In some aspects, an immunogenic composition according to the invention comprises a PCSK9 immunogen, optionally comprising a linker, linked to an immunogenic carrier, selected from keyhole limpet hemocyanin (KLH), tetanus toxoid heavy chain fragment (rTTHC), H. influenzae protein D (HiD), and cross-reactive material from diphtheria toxin (CRM197).

In some aspects, a subject immunogenic composition comprises single species of antigenic PCSK9 peptide, e.g., the immunogenic composition comprises a population of antigenic PCSK9 peptides, substantially all of which have the same amino acid sequence. In other aspects, a subject immunogenic composition comprises two or more different antigenic PCSK9 peptides, e.g., the immunogenic composition comprises a population of antigenic PCSK9 peptides, the members of which population can differ in amino acid sequence. A subject immunogenic composition can comprise from two to about 20 different antigenic PCSK9 peptides, e.g., a subject immunogenic composition can comprise 2, 3, 4, 5, 6, 7, 8, 9, 10, 11-15, or 15-20 different antigenic PCSK9 peptides, each having an amino acid sequence that differs from the amino acid sequences of the other antigenic PCSK9 peptides. In some aspects, the PCSK9 immunogen is selected from one of residue regions 153-155, 192-197, 212-217, 220-226, 237-243, 366-372, and 374-381 of PCSK9, as defined by NP_777596.2 (SEQ ID NO:86). The PCSK9 immunogen can comprise one or more of residue regions 153-155, 192-197, 212-217, 220-226, 237-243, 366-372, and 374-381 of PCSK9.

In some aspects, the PCSK9 immunogen comprises peptides of PCSK9 comprising non-native (engineered) mutations. Non-limiting examples of such motifs include :. KEDGTRFHRsASKCeS (SEQ ID NO: 147), RDAGVAr (SEQ ID NO: 148), and EDIIsASgDCSTCFVS (SEQ ID NO: 149) (Upper case letters denote human PCSK9 residues, lower case letters denote engineered mutations). These three sequence motifs are exemplary only. A review of the sequences in the tables (SEQ ID Nos: 1-146) reveals several instances in which non-native (engineered) mutations are present in the PCSK9 immunogen. The inclusion of such motifs can facilitate removal of 9-mers oligopeptides found in human protein to reduce self-targeting response.

In other aspects, a subject immunogenic composition comprises a multimerized antigenic PCSK9 polypeptide, as described above. As used herein, the terms “immunogenic composition comprising an antigenic PCSK9 peptide” or “immunogenic composition of the invention” or “subject immunogenic composition” refers to an immunogenic composition comprising either single species (multimerized or not) or multiple species of antigenic PCSK9 peptide(s) coupled or not to an immunogenic carrier. Where two or more peptides are used coupled to a carrier, the peptide may be coupled to the same carrier molecule or individually coupled to carrier molecules and then combined to produce an immunogenic composition.

Another aspect of the invention relates to methods for producing an immunogen according to the invention, the method comprising coupling an antigenic PCSK9 peptide to an immunogenic carrier. In one aspect, the coupling is chemical.

### Adjuvants

In some aspects, a subject immunogenic composition comprises at least one adjuvant. Suitable adjuvants include those suitable for use in mammals, preferably in humans. Examples of known suitable adjuvants that can be used in humans include, but are not necessarily limited to, alum, aluminum phosphate, aluminum hydroxide, MF59 (4.3% w/v squalene, 0.5% w/v polysorbate 80 (Tween 80), 0.5% w/v sorbitan trioleate (Span 85)), CpG-containing nucleic acid (where the cytosine is unmethylated), QS21 (saponin adjuvant), MPL (Monophosphoryl Lipid A), 3DMPL (3-O-deacylated MPL), extracts from Aquilla, ISCOMS (see, e.g., Sjolander et al. (1998) J. Leukocyte Biol. 64:713: WO90/03184, WO96/11711, WO 00/48630, WO98/36772, WO00/41720, WO06/134423 and WO07/026,190), LT/CT mutants, poly(D.L-lactide-co-glycolide) (PLG) microparticles, Quil A, TiterMax classic, TiterMax Gold, interleukins, and the like. For veterinary applications including but not limited to animal experimentation, one can use Freund's adjuvant, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(l′-2′-dipalmitoyl-s-n-glycero-3-hydroxy phosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™, containing 5% Squalene, 0.5% Tween 80 (polyoxyethylene sorbitan mono-oleate), and 0.5% Span 85 (sorbitan trioleate) (optionally containing muramyl tri-peptide covalently linked to dipalmitoyl phosphatidylethanolamine (MTP-PE)) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (RIBI IMMUNOCHEM, HAMILTON, Mont.) containing 2% Squalene, 0.2% Tween 80, and one or more bacterial cell wall components such as monophosphory lipid A (MPL), trehalose dimycolate (TDM), and cell wall skeleton (CWS), preferably MPL+CWS (DETOX™): (2) saponin adjuvants, such as QS21, STIMULON™ (Cambridge Bioscience, Worcester, Mass.), ABISCO® (ISCONOVA, Sweden), or ISCOMATRIX® (COMMONWEALTH SERUM LABORATORIES, Australia), may be used or particles generated therefrom such as ISCOMs (immunostimulating complexes), which ISCOMS may be devoid of additional detergent e.g. WO00/07621: (3) Complete Freund's Adjuvant (CFA) and Incomplete Freund's Adjuvant (IFA): (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.: (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO00/56358: (6) combinations of 3dMPL with, for example, QS21 and/or oil-in-water emulsions e.g. EP-A-0835318, EP-A-0735898, EP-A-0761231: (7) oligonucleotides comprising CpG motifs: International patent applications WO96/02555, WO98/16247, WO98/18810, WO98/40100, WO98/55495, WO98/37919 and WO98/52581] i.e. containing at least one CG dinucleotide, where the cytosine is unmethylated: (8) a polyoxyethylene ether or a polyoxyethylene ester e.g. WO99/52549: (9) a polyoxyethylene sorbitan ester surfactant in combination with an octoxynol (WO01/21207) or a polyoxyethylene alkyl ether or ester surfactant in combination with at least one additional non-ionic surfactant such as an octoxynol (WO01/21152): (10) a saponin and an immunostimulatory oligonucleotide (e.g. a CpG oligonucleotide) (WO00/62800): (11) an immunostimulant and a particle of metal salt e.g. WO00/23105: (12) a saponin and an oil-in-water emulsion e.g. WO99/11241: (13) a saponin (e.g. QS21)+3dMPL+IM2 (optionally+a sterol) e.g. WO98/57659: (14) other substances that act as immunostimulating agents to enhance the efficacy of the composition, such as Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxy phosphoryloxy)-ethylamine MTP-PE), (15) ligands for toll-like receptors (TLR), natural or synthesized (e.g. as described in Kanzler et al 2007, Nature Medicine 13, p1552-9), including TLR3 ligands such as polyl:C and similar compounds such as HILTONOL and AMPLIGEN. Another suitable adjuvant for use in certain embodiments of the present invention is as described, for example, in U.S. Pat. Nos. 6,676,958 and 7,879,333 and sold under the tradename ADJUPLEX.

In a particular aspect, the adjuvant is an immunostimulatory oligonucleotide and more preferably a CpG oligonucleotide. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another aspect the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

### Pharmaceutical Compositions of the Invention

The invention also provides pharmaceutical compositions comprising an antigenic PCSK9 peptide of the invention or an immunogenic composition thereof, in a formulation in association with one or more pharmaceutically acceptable excipient(s) and optionally combined with one or more adjuvants, as described above. The term ‘excipient’ is used herein to describe any ingredient other than the active ingredient, i.e. the antigenic PCSK9 peptide of the invention eventually coupled to an immunogenic carrier and optionally combined with one or more adjuvants. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the active ingredient.

Pharmaceutical compositions of the present invention and methods for their preparation will be readily apparent to those skilled in the art. Such compositions and methods for their preparation may be found, for example, in Remington: The Science and Practice of Pharmacy, 23rd Edition (Academic Press 2020). Pharmaceutical compositions are preferably manufactured under GMP conditions.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Any method for administering peptides, or proteins accepted in the art may suitably be employed for the peptides or proteins of the invention.

The pharmaceutical compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions: and kidney dialytic infusion techniques. Preferred aspects include the intravenous, subcutaneous, intradermal and intramuscular routes, even more preferred aspects are the intramuscular or the subcutaneous routes.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one aspect of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, microparticles, or in a liposomal preparation. Formulations for parenteral administration may be formulated to be immediate and/or modified release. Modified release formulations include delayed-, sustained-, pulsed-, controlled-, targeted and programmed release.

For example, in one aspect, sterile injectable solutions can be prepared by incorporating the anti-PCSK9 peptide, preferably coupled to an immunogenic carrier, optionally in combination with one or more adjuvants, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An exemplary, non-limiting pharmaceutical composition of the invention is a formulation as a sterile aqueous solution having a pH that ranges from about 5.0 to about 6.5 and comprising from about 0.1 mg/mL to about 20 mg/mL of a peptide of the invention, from about 1 millimolar to about 100 millimolar of histidine buffer, from about 0.01 mg/mL to about 10 mg/mL of polysorbate 80, from about 100 millimolar to about 400 millimolar of trehalose, and from about 0.01 millimolar to about 1.0 millimolar of disodium EDTA dihydrate.

The PCSK9 immunogens of the invention can also be administered intranasally or by inhalation, typically in the form of a dry powder (either alone, as a mixture, or as a mixed component particle, for example, mixed with a suitable pharmaceutically acceptable excipient) from a dry powder inhaler, as an aerosol spray from a pressurized container, pump, spray, atomizer (preferably an atomizer using electrohydrodynamics to produce a fine mist), or nebulizer, with or without the use of a suitable propellant, or as nasal drops.

The pressurized container, pump, spray, atomizer, or nebulizer generally contains a solution or suspension of an immunogen of the invention comprising, for example, a suitable agent for dispersing, solubilizing, or extending release of the active, a propellant(s) as solvent.

Prior to use in a dry powder or suspension formulation, the drug product is generally micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the compound of the invention, a suitable powder base and a performance modifier.

A suitable solution formulation for use in an atomizer using electrohydrodynamics to produce a fine mist may contain a suitable dose of the antigenic PCSK9 peptide of the invention per actuation and the actuation volume may vary from 1 μL to 100 μL.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” of an immunogen of the invention. The overall daily dose will typically be administered in a single dose or, more usually, as divided doses throughout the day.

A pharmaceutical composition comprising an PCSK9 immunogen may also be formulated for an oral route administration. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets: soft or hard capsules containing multi- or nano-particulates, liquids, or powders: lozenges (including liquid-filled): chews: gels: fast dispersing dosage forms: films: ovules: sprays: and buccal/mucoadhesive patches.

Liquid formulations include suspensions, solutions, syrups and elixirs. Such formulations may be employed as fillers in soft or hard capsules (made, for example, from gelatin or hydroxy propylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compositions of the invention can be used to treat, alleviate or prevent PCSK9-mediated disorders or symptoms in a subject at risk or suffering from such disorder or symptom by stimulating an immune response in the subject by immunotherapy. Immunotherapy can comprise an initial immunization followed by additional, e.g. one, two, three, or more boosters.

An “immunologically effective amount” of an antigenic PCSK9 peptide of the invention, or composition thereof, is an amount that is delivered to a mammalian subject, either in a single dose or as part of a series, which is effective for inducing an immune response against PCSK9 in the subject. This amount varies depending upon the health and physical condition of the individual to be treated, the taxonomic group of individual to be treated, the capacity of the individual's immune system to synthesize antibodies, the formulation of the vaccine, and other relevant factors.

A “pharmaceutically effective dose” or “therapeutically effective dose” is that dose required to treat or prevent, or alleviate one or more PCSK9-related disorder or symptom in a subject. The pharmaceutically effective dose depends on inter alia the specific compound to administer, the severity of the symptoms, the susceptibility of the subject to side effects, the type of disease, the composition used, the route of administration, the type of mammal being treated, the physical characteristics of the specific mammal under consideration such as health and physical condition, concurrent medication, the capacity of the individual's immune system to synthesize antibodies, the degree of protection desired, and other factors that those skilled in the medical arts will recognize. For prophylaxis purposes, the amount of peptide in each dose is selected as an amount which induces an immunoprotective response without significant adverse side effects in typical vaccines. Following an initial vaccination, subjects may receive one or several booster immunizations adequately spaced.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For example, PCSK9 immunogens or pharmaceutical composition of the invention can be administered to a subject at a dose of about 0.1 μg to about 5 mg, e.g., from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg, with optional boosters given at, for example, 1 week, 2 weeks, 3 weeks, 4 weeks, two months, three months, 6 months and/or a year later.

In some aspects, a single dose of an antigenic PCSK9 peptide or pharmaceutical composition according to the invention is administered. In other aspects, multiple doses of an antigenic PCSK9 peptide or pharmaceutical composition according to the invention are administered. The frequency of administration can vary depending on any of a variety of factors, e.g., severity of the symptoms, degree of immunoprotection desired, whether the composition is used for prophylactic or curative purposes, etc. For example, in some aspects, an PCSK9 immunogen or pharmaceutical composition according to the invention is administered once per month, twice per month, three times per month, every other week, once per week, twice per week, three times per week, four times per week, five times per week, six times per week, every other day, daily, twice a day, or three times a day. When the composition of the invention is used for prophylaxis purposes, they will be generally administered for both priming and boosting doses. It is expected that the boosting doses will be adequately spaced, or preferably given yearly or at such times where the levels of circulating antibody fall below a desired level. Boosting doses may consist of the PCSK9 immmunogen in the absence of the original immunogenic carrier molecule. Such booster constructs may comprise an alternative immunogenic carrier or may be in the absence of any carrier. Such booster compositions may be formulated either with or without adjuvant.

The duration of administration of an antigenic PCSK9 peptide according to the invention, e.g., the period of time over which an antigenic PCSK9 peptide is administered, can vary, depending on any of a variety of factors, e.g., patient response, etc. For example, an antigenic PCSK9 peptide can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

A variety of treatment methods are also contemplated by the present disclosure, which methods comprise administering an antigenic PCSK9 peptide according to the invention. Subject treatment methods include methods of inducing an immune response in an individual to self-PCSK9, and methods of preventing, alleviating or treating a PCSK9-related disorder or symptom in an individual.

In one aspect, the present invention provides a method for treating, preventing or alleviating a PCSK9-related disorder or symptom in a subject, comprising administering a therapeutically effective amount of an antigenic PCSK9 peptide of the invention, or immunogenic or pharmaceutical composition thereof, to the subject.

In another aspect, the present invention provides a method for inducing an immune response against self-PCSK9 in a subject, comprising administering a therapeutically or immunogenically effective amount of an antigenic PCSK9 peptide of the invention, or immunogenic or pharmaceutical composition thereof, to the subject.

A PCSK9 related disease or a PCSK9 mediated disease is, for example, a disease where the inhibition of PCSK9 activity or the inhibition of the interaction of PCSK9 with the LDL receptor could be beneficial.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative and palliative treatment. The subject is preferably human, and may be either male or female, of any age.

Other aspects of the invention relate to an antigenic PCSK9 peptide according to the invention or of an immunogenic composition or a pharmaceutical composition thereof, for use as a medicament, preferably in treatment, alleviation or prophylaxis of PCSK9-related disorders.

In yet another aspect, the present invention provides the use of an antigenic PCSK9 peptide of the invention or of an immunogenic composition or a pharmaceutical composition thereof, in the manufacture of a medicament, preferably for treating a PCSK9- related disorder.

In particular, the invention relates to an antigenic PCSK9 peptide of the invention, or an immunogenic or pharmaceutical composition thereof, for use as a medicament preferably in treatment, alleviation or prophylaxis of diseases associated with an elevated level of cholesterol.

In yet another aspect, the present invention provides the use of an antigenic PCSK9 peptide of the invention or of an immunogenic composition or a pharmaceutical composition thereof, in the manufacture of a medicament, preferably for lowering the LDL-cholesterol level in blood in a subject in need thereof.

In some aspects of the uses or methods of the invention, the PCSK9-related disorder is selected from the group consisting of elevated cholesterol, a condition associated with elevated LDL-cholesterol, e.g., a lipid disorder (e.g., hyperlipidemia, type I, type II, type III, type IV, or type V hyperlipidemia, secondary hypertriglyceridemia, hypercholesterolemia, familial hypercholesterolemia, xanthomatosis, lecithin:cholesterol acetyltransferase deficiency), arteriosclerotic conditions (e.g., atherosclerosis), coronary artery disease, and cardiovascular disease.

In yet another aspect, the present invention provides the use of an antigenic PCSK9 peptide of the invention or of an immunogenic composition or a pharmaceutical composition thereof, in the manufacture of a medicament for treating or alleviating diseases where an up-regulation of the LDL receptor or an inhibition of the interaction between PCSK9 and the LDL receptor is beneficial.

In yet another aspect, the present invention provides the use of an antigenic PCSK9 peptide of the invention or of an immunogenic composition or a pharmaceutical composition thereof, in the manufacture of a medicament for the treatment of Alzheimer's disease.

In other aspects of the uses or methods of the invention, the subject is a mammal, preferably a human subject.

In still other aspects of the uses or methods of the invention, the subject suffers from the PSCK9-related disorder. Alternatively, the subject is at risk of suffering from the PCSK9-related disorder, e.g., due to the presence of one or more risk factors (e.g., hypertension, cigarette smoking, diabetes, obesity, or hyperhomocysteinemia).

The PCSK9 immunogen of the invention or an immunogenic composition or a pharmaceutical composition thereof are useful for subjects who are intolerant to therapy with another cholesterol-reducing agent, or for whom therapy with another cholesterol-reducing agent has produced inadequate results (e.g., subjects who experience insufficient LDL-c reduction on statin therapy). The PCSK9 immunogen of the invention described herein can be administered to a subject with elevated LDL-cholesterol.

Preferably a subject with elevated cholesterol is a human subject with total plasma cholesterol levels of 200 mg/dl or greater. Preferably a subject with elevated cholesterol is a human subject with LDL-cholesterol levels of 160 mg/dl or greater.

Total plasma cholesterol levels and LDL-cholesterol levels are measured using standard methods on blood samples obtained after an appropriate fast. Protocols to measure total plasma cholesterol levels and LDL-cholesterol levels are well-known to persons skilled in the art.

In one aspect the PCSK9 immunogen or an immunogenic composition or a pharmaceutical composition thereof is administered together with another agent, the two can be administered sequentially in either order or simultaneously. In some aspects, an PCSK9 immunogen or an immunogenic composition or a pharmaceutical composition thereof is administered to a subject who is also receiving therapy with a second agent (e.g., a second cholesterol-reducing agent). Cholesterol reducing agents include statins, bile acid sequestrants, niacin, fibric acid derivatives, and long chain alpha, omego-dicarboxylic acids. Statins inhibit cholesterol synthesis by blocking HMGCoA, a key enzyme in cholesterol biosynthesis. Examples of statins are lovastatin, pravastatin, atorvastatin, cerivastatin, fluvastatin, and simvastatin. In some aspects, combination therapy with an PCSK9 immunogen or an immunogenic composition or a pharmaceutical composition thereof and a statin produces synergistic results (e.g., synergistic reductions in cholesterol). In some subjects, this can allow reduction in statin dosage to achieve the desired cholesterol levels.

Another aspect of the invention relates to the use of a vaccine construct of the invention in the manufacture of a medicament for the treatment of a PCSK9-related disorder or condition, as described above.

Another aspect of the invention relates to the use of an immunogenic composition of the invention in the preparation of a medicament for the treatment of a PCSK9-related disorder or condition, as described above.

Another aspect of the invention relates to a vaccine construct of the invention for use in the treatment of a PCSK9-related disorder or condition, as described above. In these aspects, the vaccine construct may be designed to be administered as a pharmaceutical composition including at least one excipient, as described above.

Other specific aspects include methods of treating a PCSK9-related disorder or condition, as described above, in an individual comprising administering an effective amount of a vaccine construct of the invention to an individual in need thereof.

Each publication or patent document cited in this disclosure is incorporated herein by reference in its entirety.

The invention now being generally described will be more readily understood by reference to the following examples, which are included merely for the purposes of illustration of certain aspects of the aspects of the present invention. The examples are not intended to limit the invention, as one of skill in the art would recognize from the above teachings and the following examples that other techniques and methods can satisfy the claims and can be employed without departing from the scope of the claimed invention.

### EXAMPLES

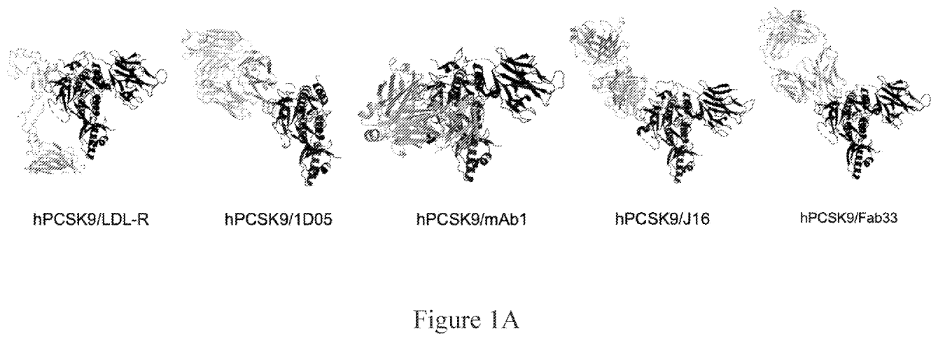

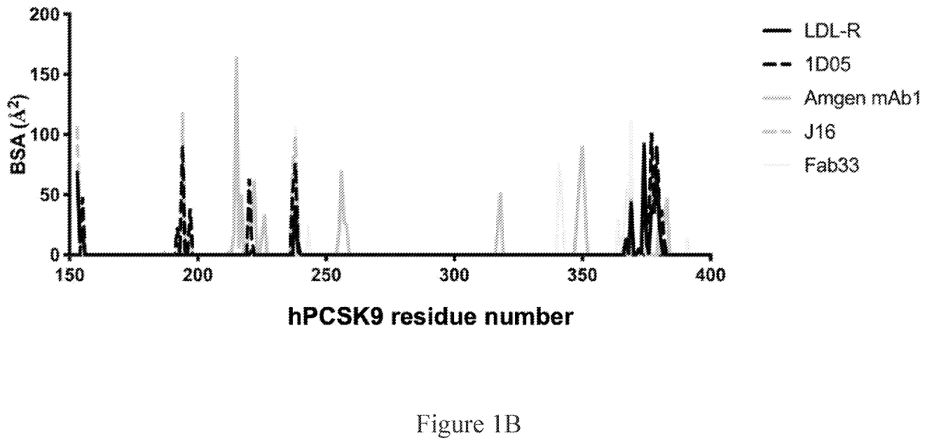

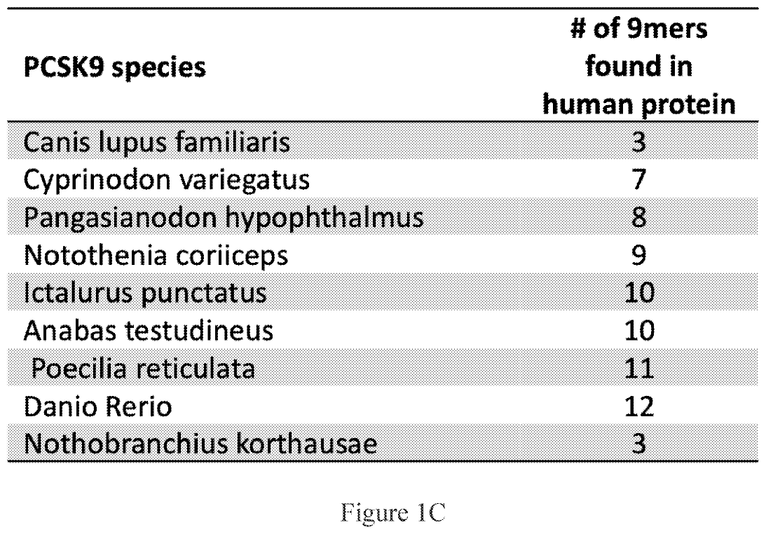

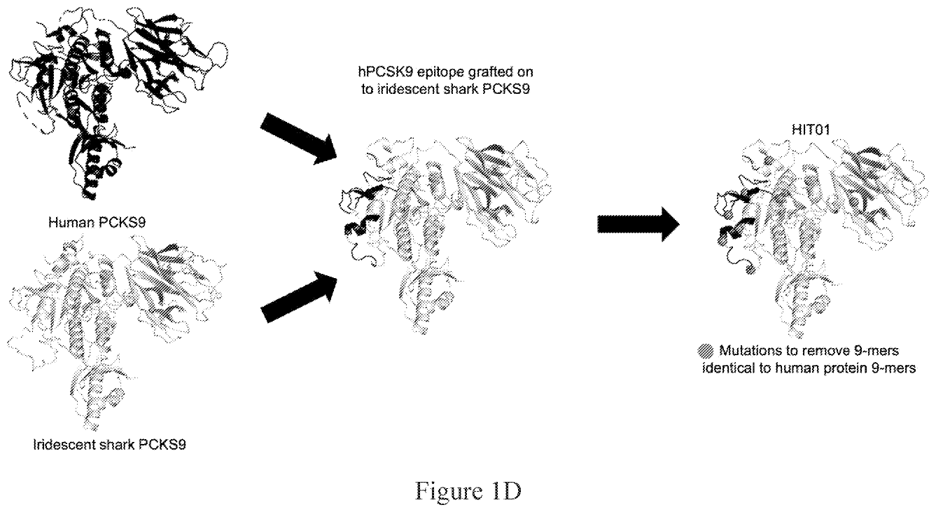

### Example 1: Design of PCSk9 Mimic Immunogens. (FIGS. 1A-1D)

To identify the epitope residues for antibodies targeting human PCSK9, buried surface area was calculated between human PCSK9 and LDL-R, and between human PCSK9 and antibodies 1D05, mAb1, J16, or Fab33 using NACCESS. Human PCSK9 residues that contact multiple antibodies/LDL-R, such as the following, were grafted on various scaffold proteins:
153-155
192-197
212-217
220-226
237-243
366-372
374-381

To identify the scaffold to graft the Human PCSK9 residues, non-human PCSK9 deposited at the NCBI (ncbi.nlm.nih.gov) was identified and the DALI webserver (ekhidna2.biocenter.helsinki.fi/dali/) was used to identify structural homologs to Human PCSK9. Once the Human PCSK9 epitope residues were grafted on to the scaffold sequence based on sequence alignment, 9-mers identical to human protein 9-mers were identified, by comparing all 9-mer of the epitope-scaffold to all 9-mers of all human (Build 37, hg19 (February 2009) from the International Human Genome Consortium). For 9-mers identical to human 9-mers, mutations were made to remove this identity and choice of mutations were based on minimizing the impact of the mutation to antibody binding or stability of the protein.

### Example 2: Antigenic Screening to Identify PCSk9 Mimic Immunogens With Desired Antigenic Profile

Logarithmic growth cell preparation: HEK 293T cells (Invitrogen, CA) were thawed and incubated with Growth Medium (DMEM with 10% Fetal Bovine Serum and 1% streptomycin-penicillin) at 37° C., 5% CO₂. The cells are cultured under logarithmic growth conditions, and passed by dislodging the cells by gently tapping the side of the flask until the cells reached logarithmic growth status.

Seeding: 24 hours prior to DNA-transient transfection, 100 μl of logarithmic growth cells were seeded in each well of a 96-well microplate at a density of 2.5×10⁵ cells/ml (prepared with 0.05% Trypsin-EDTA separation), spun down at 200×g for 6 min, and resuspended in optimized expression medium (REALFECT-Medium, ABI SCIENTIFIC, VA), and incubated at 37° C., 5% CO₂ for 24 hours (cell confluence: ˜80%). Prior to transfection, 40 μl of spent medium in each well was removed without replacement.

Transfection: DNA-TRUEFECT-MAX complexes were prepared by mixing 0.25 μg DNA in 10 μl of Opti-MEM (Invitrogen) with 0.75 μl of TRUEFECT-MAX (UNITED BIOSYSTEMS, Bethesda, MD) in 10 μl of Opti-MEM, and incubating for 15 min prior to transfection. 20 μl of the complex was added into each well and gently mixed manually (or agitated at 40 rpm on a shaker), and then the 96-well plate was incubated at 37° C., 5% CO₂. During incubation, the plate was gently mixed hourly a couple of times.

Feeding: 6 hours to 20 hours post-transfection, 30 μl of enriched feed medium (CELL GROWTH ENHANCER FOR ADHERENT CELLS, ABI SCIENTIFIC, VA) was added to each well. (Option: during incubation, gently mixing the incubated 96-well plate daily.)

Antigenic assay: After incubation for four days post-transfection, the antigenic analysis of PCKS9 immunogens was characterized by 96-well-formatted ELISA. Briefly, 30 μl per well of the expressed immunogen supernatant mixed with 70 μl per well of PBS was incubated in a Nickel coated 96-well plate (Thermo Scientific, IL) for two hours at room temperature (RT). After washing. 100 μl per well of 10 μg/ml primary antibody in 50% CELBOOSTER CELL GROWTH ENHANCER MEDIUM and PBS with 0.02% tween 20 was incubated for 1 hour at RT. After washing, 100 μl per well of Horseradish peroxidase (HRP)-conjugated goat anti-human IgG antibody (JACKSON IMMUNORESEARCH LABORATORIES INC., PA), diluted at 1:10,000 in CELBOOSTER CELL GROWTH ENHANCER MEDIUM with 0.02% tween 20, was incubated for 30 min at RT. After washing, the reaction signal was developed using 100 μl of BioFX-TMB (SURMODICS, MN) at RT for 10 min, and then stopped with 100 μl of 0.5 N H₂SO₄. The readout was measured at a wavelength of 450 nm, and OD₄₅₀ values were normalized and analyzed. All samples were performed in duplicate.

For the antigenic analysis of PCSK9 variants, following primary antibodies that inhibit PCKS9 function were assessed: AMG145, J16, Fab33, Alirocumab, 1D05, mAb1, and antibodies for negative control: Mota, VRC01, and antibody used to quantitate the expression levels of immunogens: anti-Strep mAb.

HIT01—The leading candidate, HIT01 (SEQ ID NO: 1), also termed “PCSK9 HIT01”, was based on grafting of human PCSK9 epitope residues on to iridescent shark PCSK9, with 9-mers identical to human 9-mers removed.

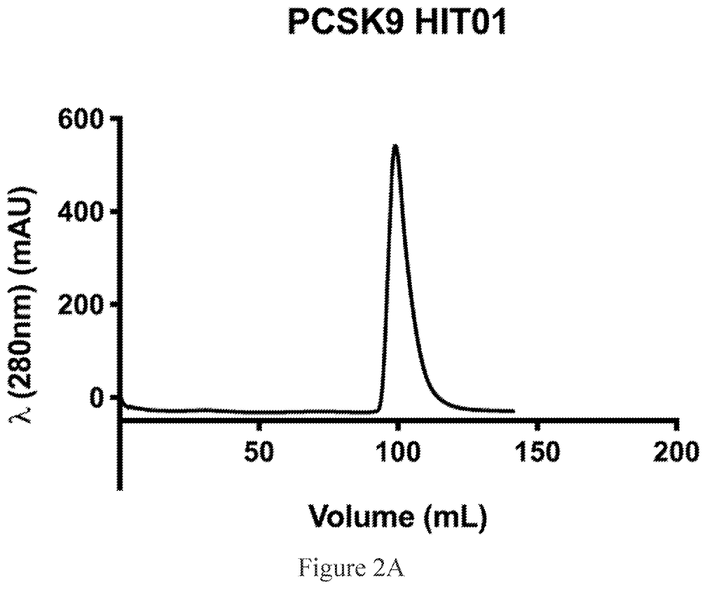

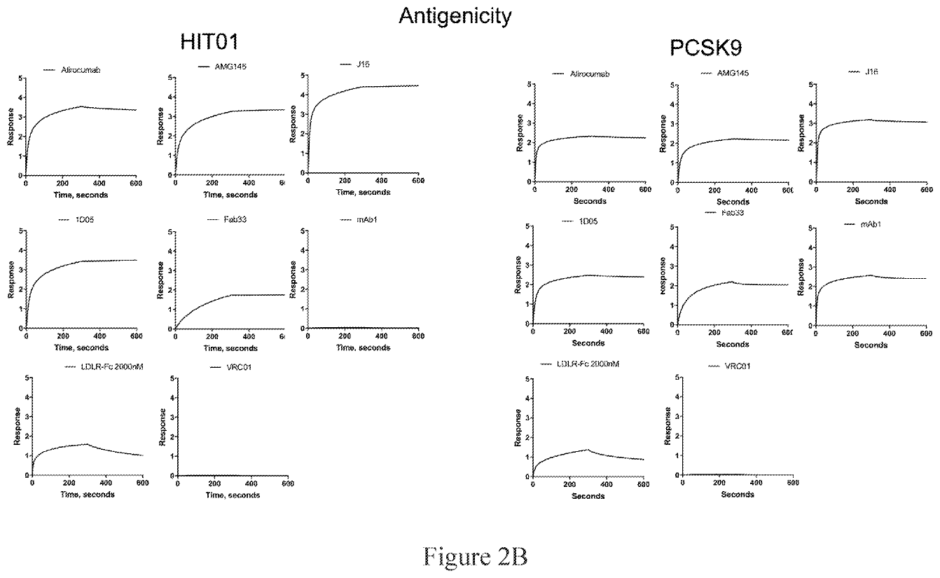

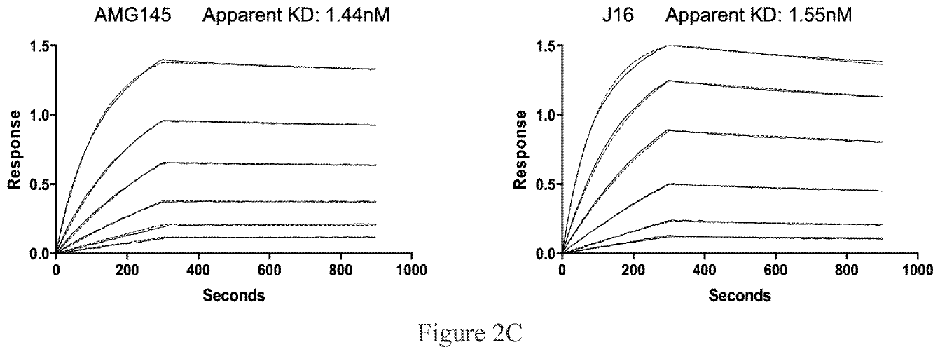

### Example 3: Protein Production and Purification (FIG. 2A)

The DNA constructs for protein expression were synthesized and subcloned in pVRC8400 expression vector. For protein expression, 3 ml of TURBO293 transfection reagent (SPEED BIOSYSTEMS) were mixed with 50 ml OPTI-MEM medium (LIFE TECHNOLOGY) and incubated at room temperature for 5 min. 1 mg plasmid DNA was mixed with 50 ml of OPTI-MEM medium in a separate tube, and the mixture were added to the TURBO293 OPTI-MEM MIXTURE. The transfection mixture was incubated for 15 min at room temperature then added to 800 ml of EXPI293 cells (LIFE TECHNOLOGY) at 2.5 million cells/ml. The transfected cells were incubated overnight in a shaker incubator at 9% CO₂, 37° C., and 120 rpm. On the second day, about 100 ml of EXPI293 expression medium was added. On day 5 post transfection, supernatants were harvested and filtered. Proteins were purified from the supernatant using Ni-NTA and strep chromatography, consecutively, followed by size exclusion column on SUPERDEX 200 INCREASE 10/300 GL in PBS. FIG. 2A illustrates the size-exclusion chromatography (SEC) profile of PCSK9-mimic HIT01.

### Example 4: Antigenicity Analysis (FIGS. 2B-2C)

A fortéBio Octet HTX instrument was used to measure antibody recognition of hPCSK9. Specific binding responses were obtained using Ni-NTA sensor tips and sera dilutions in the range of 1:200 in PBS supplemented with 1% BSA. Assays were performed at 30° C. in tilted black 384-well plates (GEIGER BIO-ONE) with agitation set to 1,000 rpm in PBS supplemented with 1% BSA and a well volume of 55 μl. All experiments were performed in duplicate.

hPCSK9 probes (20 μg/ml) were immobilized for 300 s on Ni-NTA biosensor tips. The association after 300 s was recorded. For antibody blocking assay, sensor tips were loaded with 20 μg/ml blocking antibody for 300 s. Data analysis was performed using Octet data analysis program. Data are reflected in FIG. 2B.

In a separate experiment, for AMG145 and J16, FIG. 2C illustrates the apparent KD of HIT01 binding as determined by using Octet, solid lines are observed response curves and dotted lines are fitting curves. The results indicate the designed PCSK9 mimic immunogen HIT01 contains epitopes that are recognized by a panel of monoclonal antibodies, which have been shown to reduce cholesterol when passively infused, such as mAb1 and AMG145: Schiele F. et al, JMB 2013 (pubmed.ncbi.nlm.nih.gov/24252255/), Alirocumab: Gusarova V. et al, Clin Lipidol. 2012 (tandfonline.com/doi/pdf/10.2217/clp. 12.70), J16: Liang H, et al, Journal of Pharmacology and Experimental Therapeutics, 2012 (jpet.aspetjournals.org/content/340/2/228), Fab 33: Zhang Y. et al, Nature Structural & Molecular Bio. (nature.com/articles/nsmb.3453?proof=t), and 1D05: Ni Y.G. et al, J Lipid Res. 2011 (pubmed.ncbi.nlm.nih.gov/20959675/).

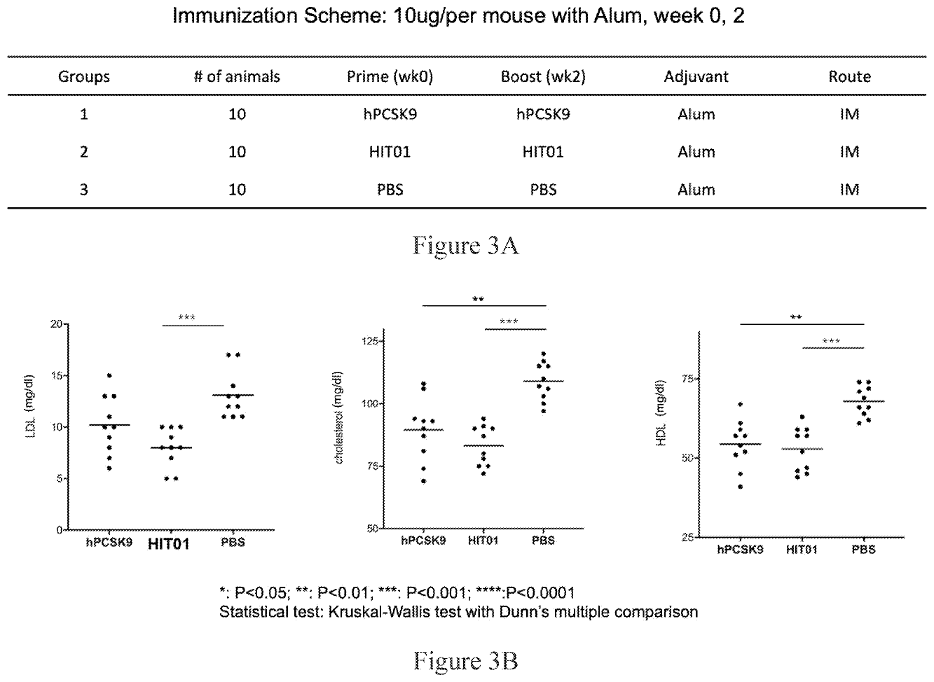

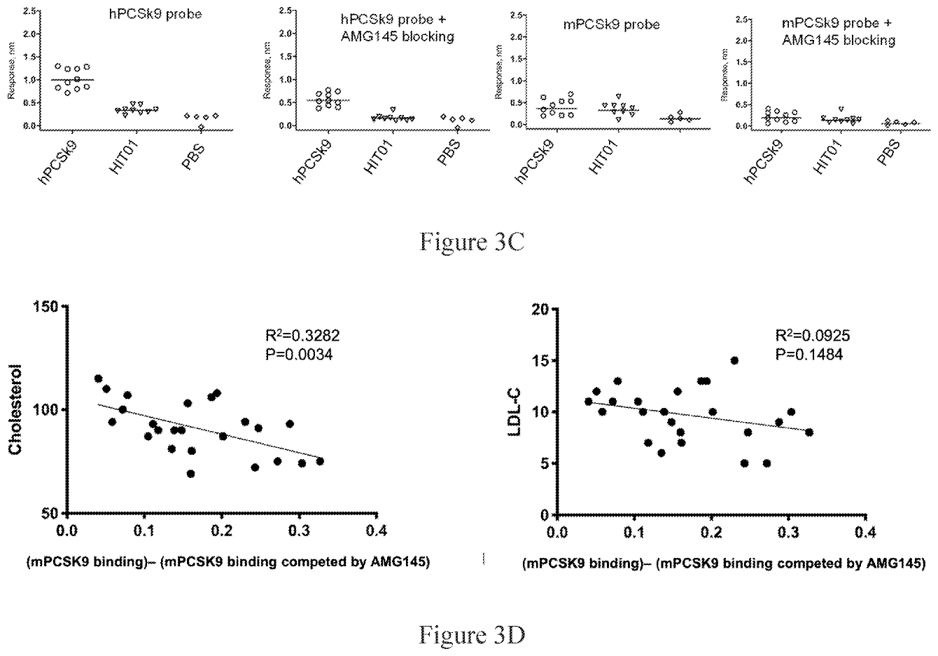

### Example 5: Mouse Immunization Study (FIGS. 3A and 3B)

Groups of 10 Balb/cJ mice were immunized 2 times in two-week intervals. For each immunization, 10 μg human wt PCSK9, HIT01, or PBS formulated with 100 μg of Aluminum hydroxide gel in the final volume of 100 μL were injected intramuscularly to the caudle thigh of the two hind legs.

Serum samples were taken six weeks after the last immunization. The results (FIG. 3B) reveal that the PCSK9-mimic HIT01 significantly reduced LDL-C, HDL-C, and cholesterol compared to the PBS group.

### Example 6: Sera Binding Analysis (FIGS. 3C and 3D)

A fortéBio Octet HTX instrument was used to measure sera recognition of PCSK9. Specific binding responses were obtained using Ni-NTA sensor tips and antibody dilutions in PBS OCTET KINETICS BUFFER (FORTÉBIO). Assays were performed at 30° C. in tilted black 384-well plates (GEIGER BIO-ONE) with agitation set to 1,000 rpm in OCTET KINETICS BUFFER and a well volume of 55 μl. PCSK9 probes (20 μg/ml) were immobilized for 300 s on Ni-NTA biosensor tips and equilibrated for 60 s prior to measuring serum samples from immunized animals at 200 fold dilution. Data analysis was performed using OCTET and GRAPHPAD PRISM 6 software.

FIG. 3C illustrates the sera antibody Octet binding response of hPCSK9, hPCSK9 HIT01, and a PBS control against hPCSK9 and mPCSK9, with and without competition by antibody AMG145. FIG. 3D illustrates the correlation of sera response targeting AMG145 epitope, as defined by mPCSK9 Octoberet binding minus mPCSK9 Octoberet binding when competed by antibody AMG145 for cholesterol (left) and LDL-C (right). The results demonstrated LDL-R competitive antibodies are elicited in HIT01-immunized animals.

### Example 7: Inhibition of LDL-R/hPCSK9 Interaction by Immunized Sera. (Prophetic)

50 microgram/ml of hPCSK9 protein (HIS tagged) were loaded onto OCTET Ni-NTA sensors. The hPCSK9-loaded sensors were then dipped into 100-300× dilution of serum collected from immunized animals. These sensors were then dipped into baseline buffer and 2000 nM of LDL-R (with human Fc tag) respectively. The response levels of both serum antibody and LDL-R were analyzed by using OCTET DATA analysis program. LDL-R competition activity was calculated as the percentage ration of LDL-R response compared to serum antibody response.

### Example 8: Quantification of Amount of PCSK9 in Sera. (Prophetic)

PCSK9 in vitro SIMPLESTEP ELISAR (Enzyme-Linked Immunosorbent Assay) kit is used for the quantitative measurement of mouse PCSK9 in serum. Briefly, the SIMPLESTEP ELISAR employs an affinity tag labeled capture antibody and a reporter conjugated detector antibody which immunocaptures the sample analyte in solution. This entire complex (capture antibody/analyte/detector antibody) is in turn immobilized via immunoaffinity of an anti-tag antibody coating the well. To perform the assay, samples or standards are added to the wells, followed by the antibody mix. After incubation, the wells are washed to remove unbound material. TMB Development Solution is added and during incubation is catalyzed by HRP, generating blue coloration. This reaction is then stopped by addition of Stop Solution completing any color change from blue to yellow. Signal is generated proportionally to the amount of bound analyte and the intensity is measured at 450 nm. Optionally, instead of the endpoint reading, development of TMB can be recorded kinetically at 600 nm.

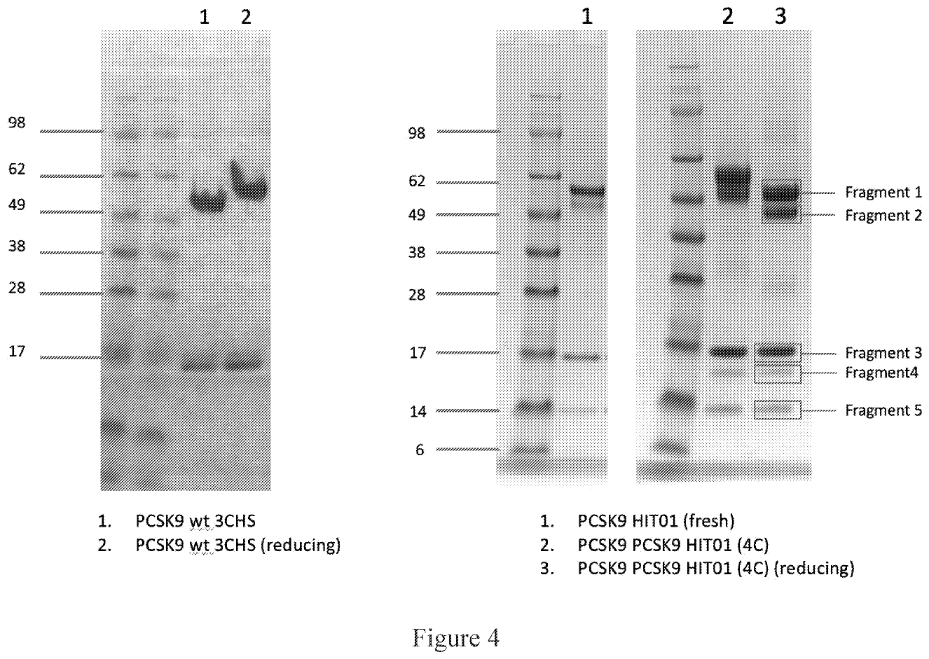

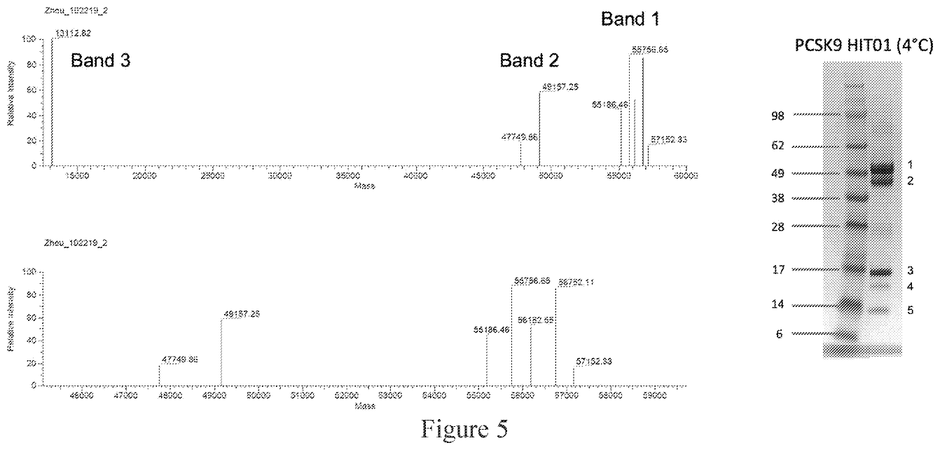

### Example 9: Proteolytic Digestion of PSCK9-Mimicking Immunogen “PCSK9 HIT01” (FIGS. 4 and 5)

PCSK9) and PCSK9 HIT01 samples were tested for proteolytic cleavage as indicated in FIG. 4 either as fresh samples or incubated at 4° C. Thereafter, the protein samples were run on gel and stained. The results. as depicted in FIG. 4. revealed that. whereas the PCSK9 shows two major bands at (a) between 49 kDa and 62 kDa and (b) at approximately 17 kDa. the PCSK9 HIT01 samples resulted in five discrete proteolytic bands. representing five distinct products of proteolytic cleavage. Based on a sequence analysis (see. e.g., SEQ ID Nos: 2-4). it was predicted that proteolytic cleavage of the PCSK9 HIT01 protein would occur at the site “FAQSIP” within the sequence. resulting in two products of MW 12994.86 and 59284.28, respectively:

DYSEDKEVKAPQLDHPDPGTERVAELLRCTKSVWRIPEQYLVVLREGTRDSHVQRT VSTLRAQAARRGHAIHIMHTYSGVFHGFLIKMSSEVLPMALKLPHVAYIEEDSSIFAQ (SEQ ID NO:152), having MW 12994.83 and
SIPWNLqRIIQTKHETGKYTPPNDGAQVTVFLLDTSVQTDHREIEGKVMVTDFNSMPK EDGTRFHRsASKCeSHGTHIAGVLSGRDAGVArGVSVNTVRVLNCQGRGTVSGALAG LEYIRASLQAQPVSPVIILLPFVGGFSRTLNTACREMVHSGAVLIAAAGNYQDDACM YSPASEPEVITVGASNAADRPLSSGTTGTNLGRCVDVFAPGEDIIsASgDCSTCFVSMS GTSQsAAHaAGIAAVLLNAYPSASPAEVLQLLRYHAVQRVINPDSLPPEHYLTTPDMV AALPTSAATGEKLLCRSVWSKRSGVGSFDTAVARCRHGEEMFSCSSYSPNGVHAGE RIEIRDGQKVCEAHHGIGGQGVYAvARCCTGSRVKCHASASLHVGIDAECPSQEFQL TGCSSHYIRSQDVAQPSWPLHSNRKACPAGEGGTSHAFCCHAPNLECHLIEHHQSEF TKQVEVSCEDSWTLTGCNAVSHGSVTHAAYTRGNTCVIQMFGGDKGAAAIAICCRY RPLDQQSNNNHEQNTgglevlfqgpghhhhhhsawshpqfek (SEQ ID NO: 153), having MW 59284.28. Accordingly, the observation that the proteolysis of the pro-protein of PCSK9 HIT01 resulted in five identifiable products revealed that it was not cleaved as expected. resulting from multiple off-target cleavages in the PCSK9 HIT01 immunogen.

The bands were separated on SDS-PAGE gel and then were cut out for N-terminal protein sequencing analysis. The sequences of the constituents of the bands determined to be as follows:

Band I, with tags MW 59284.28, = band 2 + 5:
(SEQ ID NO: 154)
SIPWNLqRIIQTKHETGKYTPPNDGAQVTVFLLDTSVQTDHREIEGKVMVTDFNSMPK
EDGTRFHRsASKCeSHGTHIAGVLSGRDAGVArGVSVNTVRVLNCQGRGTVSGALAG
LEYIRASLQAQPVSPVIILLPFVGGFSRTLNTACREMVHSGAVLIAAAGNYQDDACM
YSPASEPEVITVGASNAADRPLSSGTTGTNLGRCVDVFAPGEDIIsASgDCSTCFVSMS
GTSQsAAHaAGIAAVLLNAYPSASPAEVLQLLRYHAVQRVINPDSLPPEHYLTTPDMV
AALPTSAATGEKLLCRSVWSKRSGVGSFDTAVARCRHGEEMFSCSSYSPNGVHAGE
RIEIRDGQKVCEAHHGIGGQGVYAvARCCTGSRVKCHASASLHVGIDAECPSQEFQL
TGCSSHYIRSQDVAQPSWPLHSNRKACPAGEGGTSHAFCCHAPNLECHLIEHHQSEF
TKQVEVSCEDSWTLTGCNAVSHGSVTHAAYTRGNTCVIQMFGGDKGAAAIAICCRY
RPLDQQSNNNHEQNTgglevlfqgpghhhhhhsawshpqfek,
Band 2, most possibly is the C-term portion of Band I, 51689.76:
(SEQ ID NO: 155)
sASKCeSHGTHIAGVLSGRDAGVArGVSVNTVRVLNCQGRGTVSGALAGLEYIRASL
QAQPVSPVIILLPFVGGFSRTLNTACREMVHSGAVLIAAAGNYQDDACMYSPASEPE
VITVGASNAADRPLSSGTTGTNLGRCVDVFAPGEDIIsASgDCSTCFVSMSGTSQsAAH
aAGIAAVLLNAYPSASPAEVLQLLRYHAVQRVINPDSLPPEHYLTTPDMVAALPTSAA
TGEKLLCRSVWSKRSGVGSFDTAVARCRHGEEMFSCSSYSPNGVHAGERIEIRDGQK
VCEAHHGIGGQGVYAvARCCTGSRVKCHASASLHVGIDAECPSQEFQLTGCSSHYIR
SQDVAQPSWPLHSNRKACPAGEGGTSHAFCCHAPNLECHLIEHHQSEFTKQVEVSCE
DSWTLTGCNAVSHGSVTHAAYTRGNTCVIQMFGGDKGAAAIAICCRYRPLDQQSNN
NHEQNTgglevlfqgpghhhhhhsawshpqfek,
Band 3, MW: 12994.83:
(SEQ ID NO: 156)
DYSEDKEVKAPQLDHPDPGTERVAELLRCTKSVWRIPEQYLVVLREGTRDSHVQRT
VSTLRAQAARRGHAIHIMHTYSGVFHGFLIKMSSEVLPMALKLPHVAYIEEDSSIFAQ,
Band 4, cleavage product of band 3, cleaved after a Lys:
(SEQ ID NO: 157)
EVKAPQLDHPDPGTERVAELLRCTKSVWRIPEQYLVVLREGTRDSHVQRTVSTLRAQ
AARRGHAIHIMHTYSGVFHGFLIKMSSEVLPMALKLPHVAYIEEDSSIFAQ,
and
Band 5, (N-term portion of Band I, cleavage product of band 1..., 7612.54):
(SEQ ID NO: 158)
SIPWNLqRIIQTKHETGKYTPPNDGAQVTVFLLDTSVQTDHREIEGKVMVTDFNSMPk
EDGTRFHR.

Accordingly, reviewing the sequences of band 1 (beginning with SIP) and band 4 (ending with FAQ) indicates that, despite the observation of non-specific proteolytic digestion, the proprotein did exhibit appropriate processing at the FAQ/SIP location. Lysine (K21) was identified as a potential cleavage site, as was R218. Accordingly, two variants were designed to assess whether the observed non-specific proteolytic cleavage could be mitigated: these are identified as HIT01-K21Q-R218Q (SEQ ID NO: 13) and HIT01-K21Q- R218E (SEQ ID NO:14).

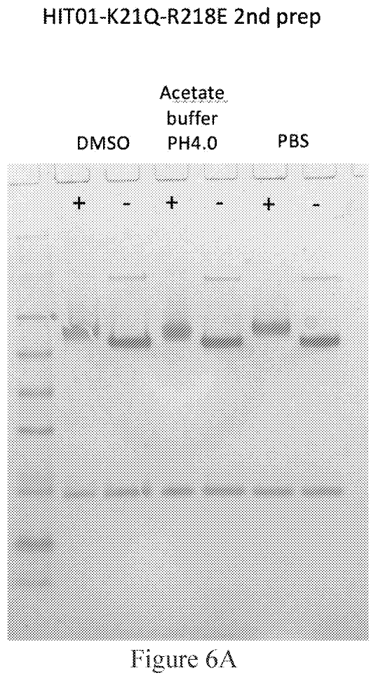

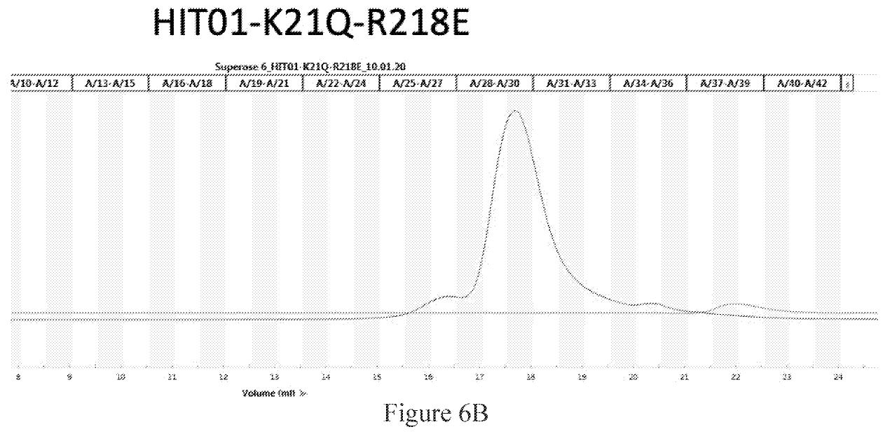

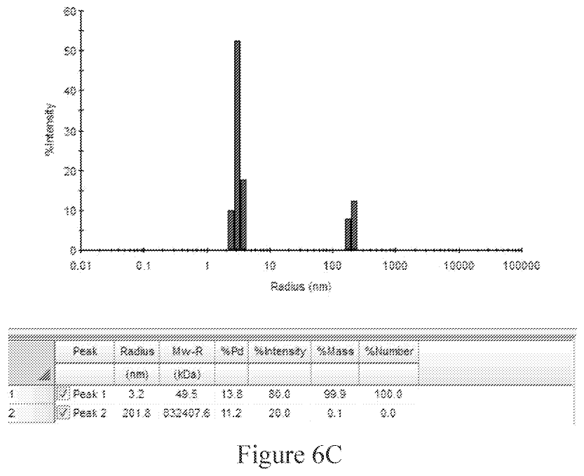

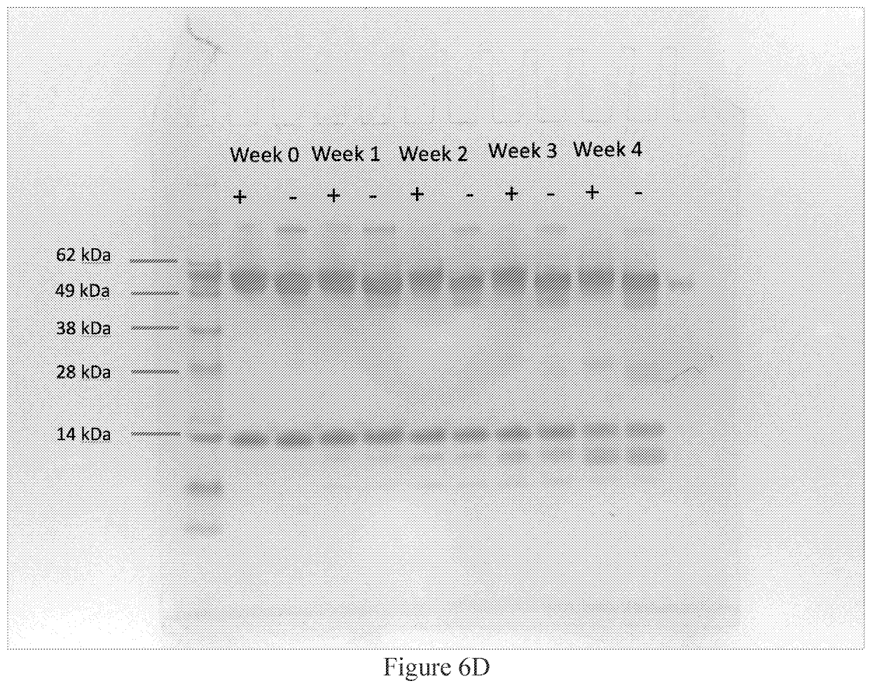

### Example 10—Stability and Binding Affinity of HIT01-K21Q-R218E (FIGS. 6A Through 6D)

The stability of HIT01-K21Q-R218E (SEQ ID NO:14) was assessed (see FIGS. 6A through 6D). FIG. 6A is SDS-PAGE data obtained at different protein storage buffer condition. The results indicate the upper band shift between reducing and non-reducing condition is consistent at various buffers. FIG. 6B is the profile of HIT01-K21Q-R218E analyzed on a size exclusion chromatography column. FIG. 6C illustrates the size distribution of HIT01-K21Q-R218E protein analyzed on a dynamic light scattering instrument. FIG. 6D is DSD_PAGE data for fresh HIT01-K21Q-R218E and at one-week intervals for four successive weeks. The results show that the HIT01-K21Q-R218E protein is stable for several weeks, although some progressive degradation is observed over the four-week period.

The binding affinity of HIT01-K21Q-R218E was assessed in comparison to unmodified PCSK9 HIT01. Briefly, antibodies (AMG145, Alirocumab, J16, Fab33, and 1D05) were separately loaded at 50 μg/ml on AHC sensors, and then dipped into PCSK9 HIT01, HIT-01 combo3 (representing a combination of mutations to reduce the amount of 8-mer and 7-mer human sequences in PCKS9), and HIT01-K21Q-R218E at 500 nm and 2× serial dilutions. The binding affinity was then calculated using the Octet Data Analysis program. The results are presented in the following table (“HIT01” refers to “PCSK9 HIT01”):

Binding Affinity KD (nM)

| Antibody IgG | HIT01 | HIT01-Combo3 | HIT01-K21Q-R218E |
|---|---|---|---|
| AMG145 | <0.001 | 0.005 | <0.001 |
| Alirocumab | 97.3 | 112.2 | 58.0 |
| J16 | 0.2 | 1.2 | 0.4 |
| Fab33 | 9.4 | 20.7 | 15.3 |
| 1D05 | 0.2 | 1.2 | 0.5 |

These results show that HIT01-K21Q-R218E retains the antigenicity similarly to its parent protein HIT01. The K21Q and R218E mutations have no significant impact on immunogenicity of HIT01 as an immunogen.

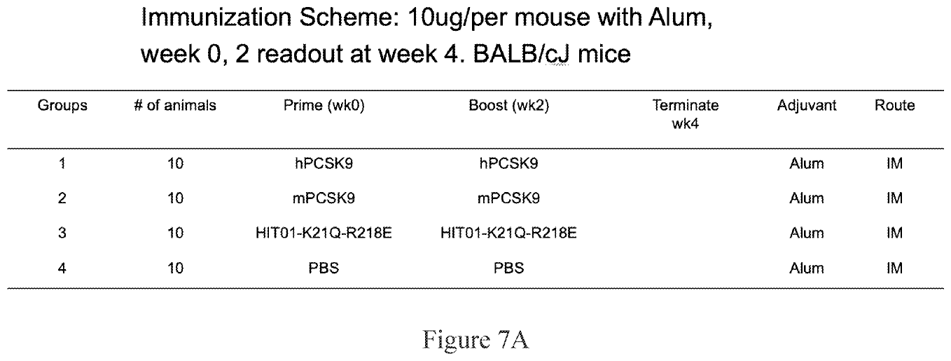

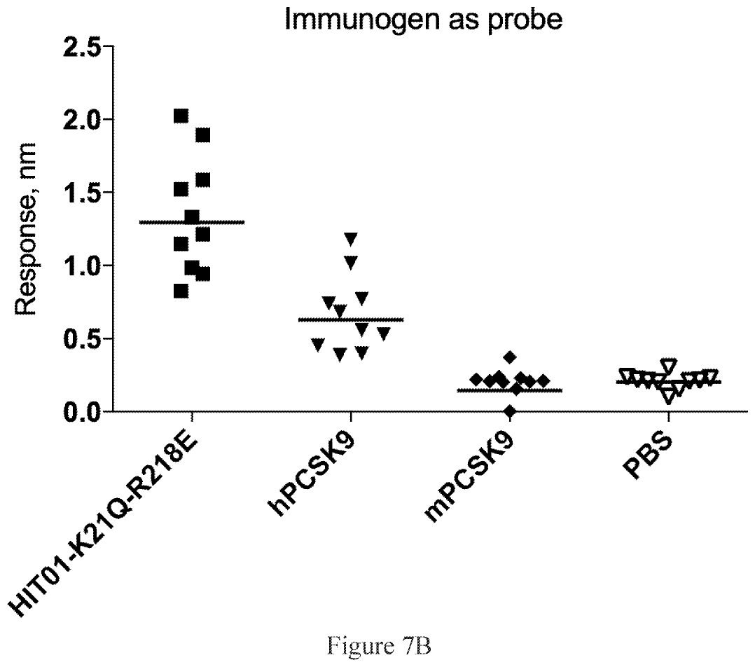

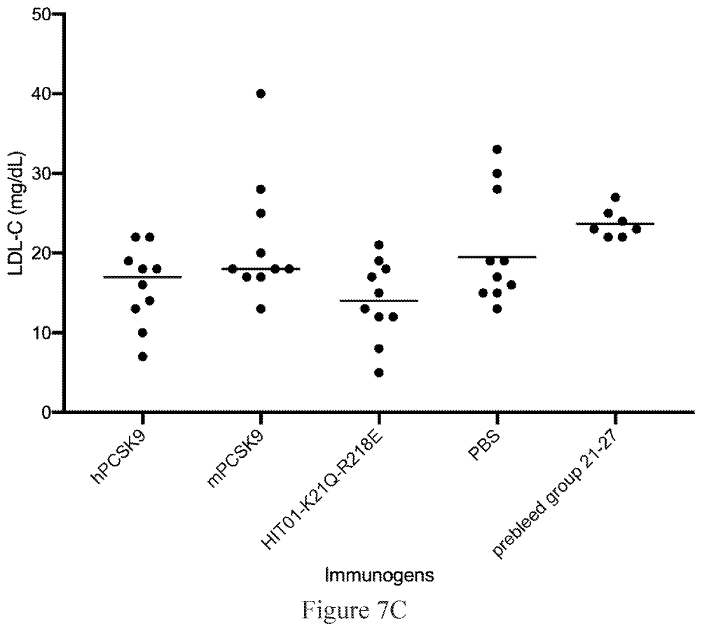

### Example 11—Mouse Immunization Study With HIT01-K21Q-R218E (FIGS. 7A Through 7C)

A mouse immunization study was conducted to assess the effect of HIT01-K21Q-R218E in comparison to wilt type human PCSK99, wild type mouse PCSK9, as well as a PBS control. The protocol is outlined in FIG. 7A and was similar to the immunization study discussed above in Example 5.

One result of the study is depicted in FIG. 7B. Terminal bleed sera from the mice in the study (Group 3 from FIG. 7A) was diluted 200-fold in 1% BSA PBS. 50 μg/ml of the indicated immunogen was immobilized on a Ni-NTA sensor. For antibody blocking, 50 μg/ml was loaded onto the immunogen-loaded sensor. The results indicated that the best recognition of this sera is to HIT01-K21Q-R281E (first column on left). With some reactivity to human PCKS9 (hPCSK9 shown in the 2nd column from the left). The other columns show different reactivity of this sera to the different immunogens.

Another result of the study is depicted in FIG. 7C, which depicts the LDL-C level in the mice treated with HIT01-K21Q-R218E, wild type human PCSK9, wild type mouse PCSK9, as well as a PBS control. The analysis was conducted in accordance with a two-tailed Mann-Whitney test. The results in FIG. 7C show that HIT01QE can reduce LDL-C slightly more than human PCSK9 (first column), and substantially better than mouse PCSK9 (second column) or PBS (fourth column). However, the range of LDL-C was too variable and there was no statistical difference between these groups.

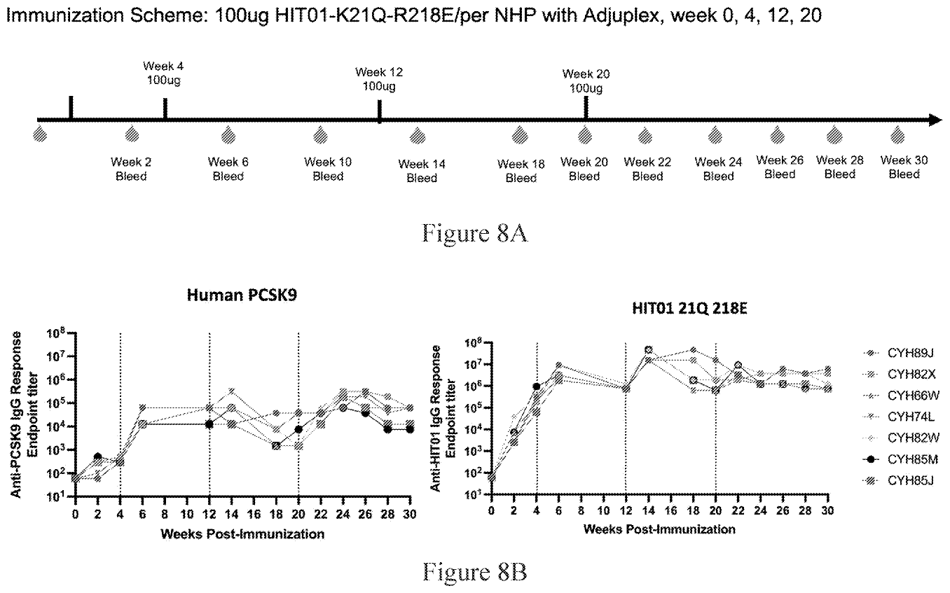

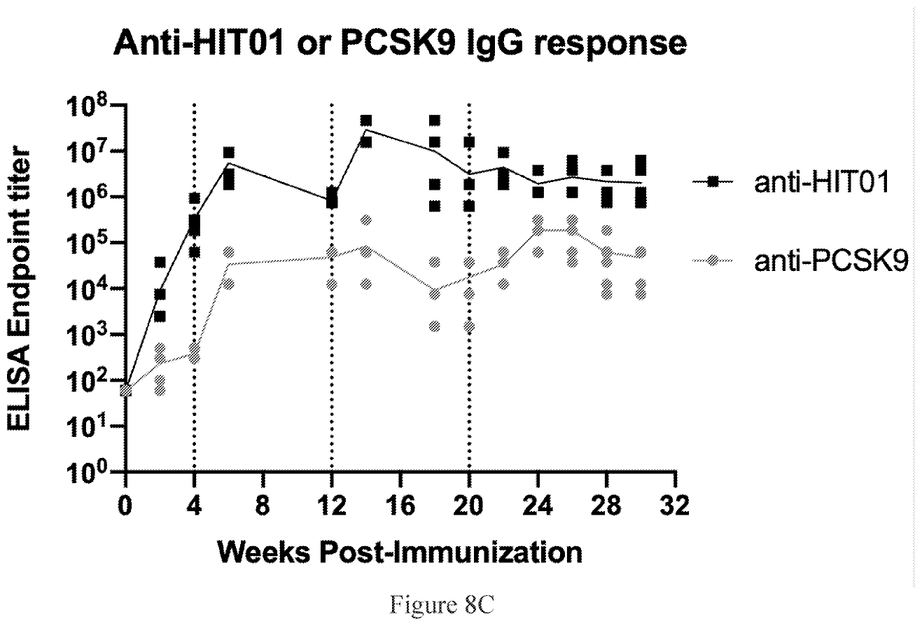

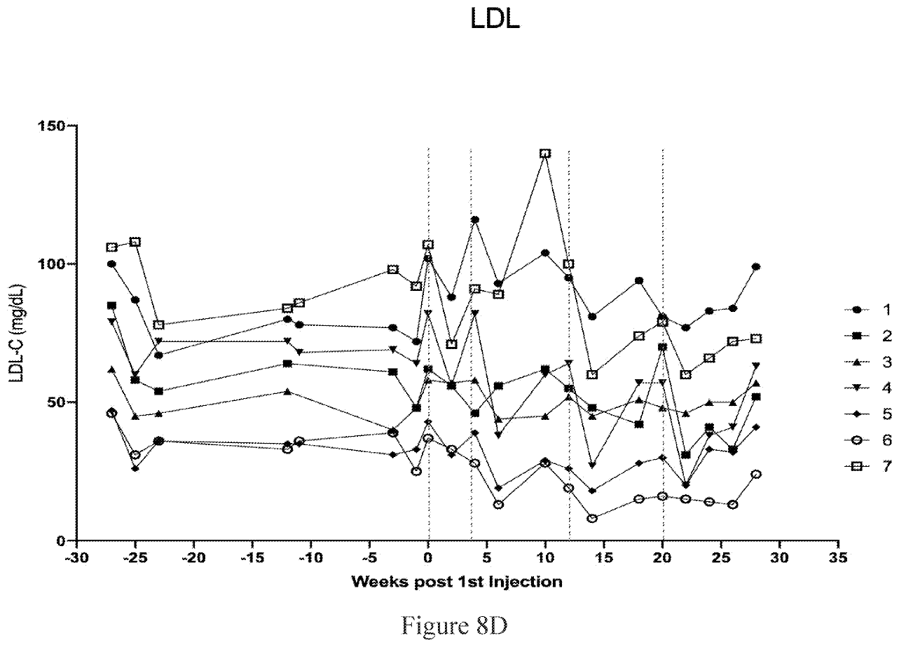

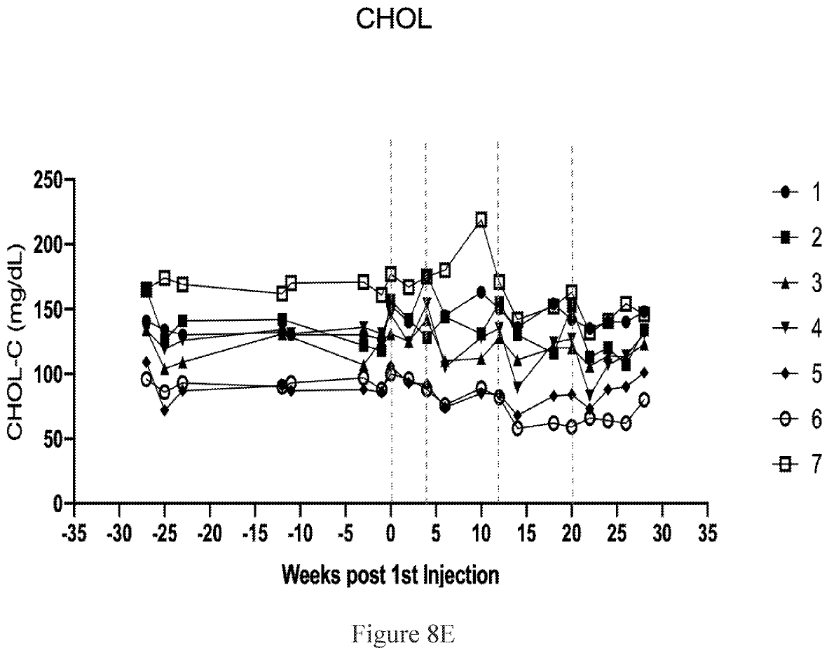

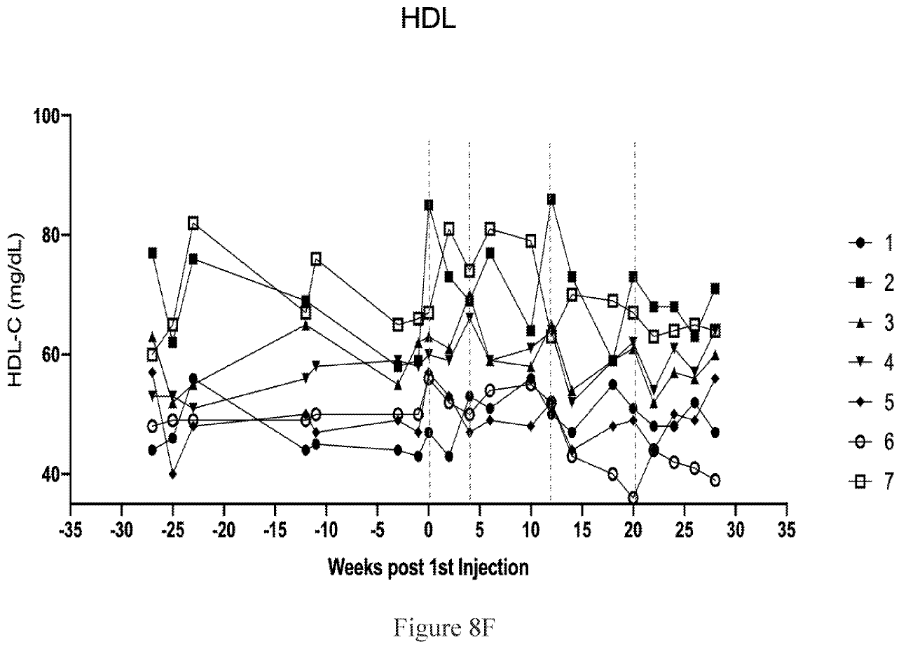

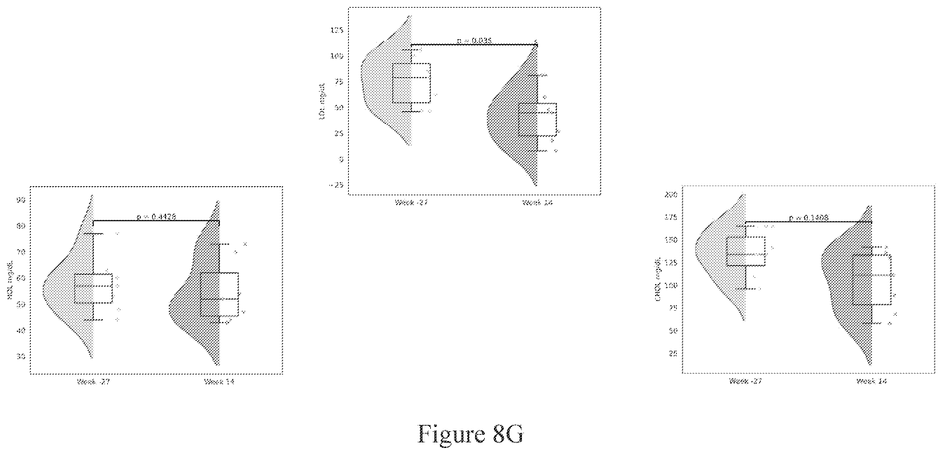

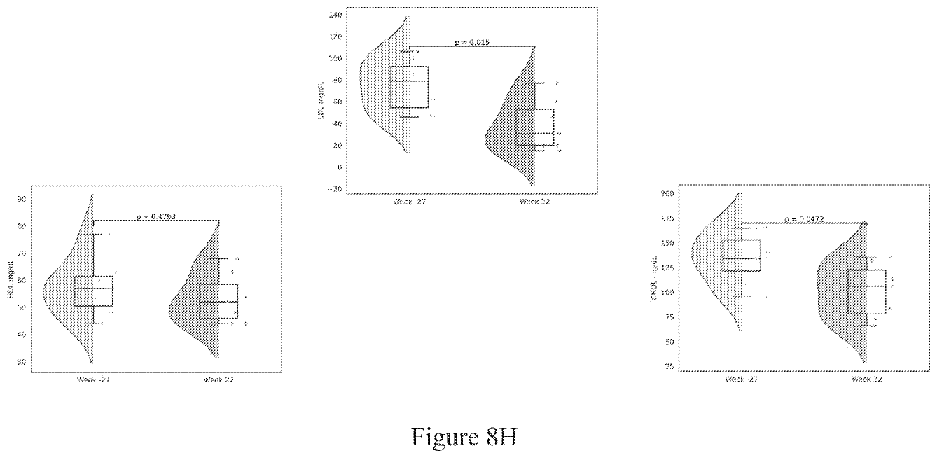

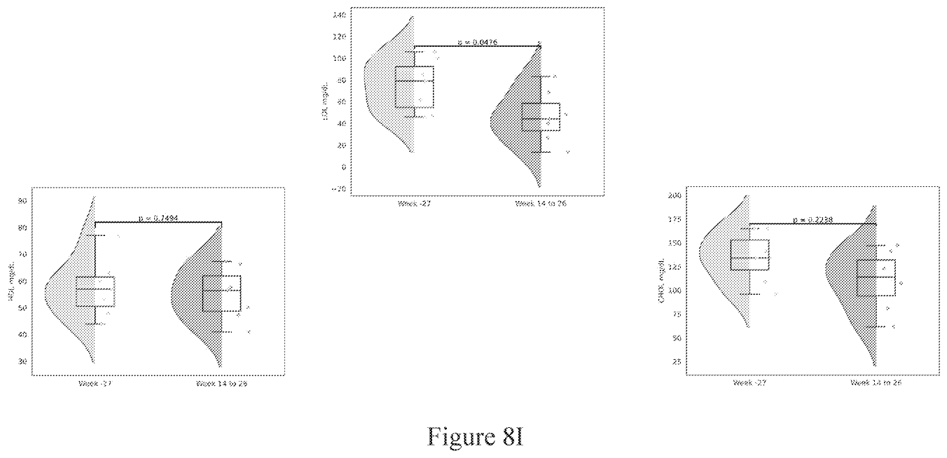

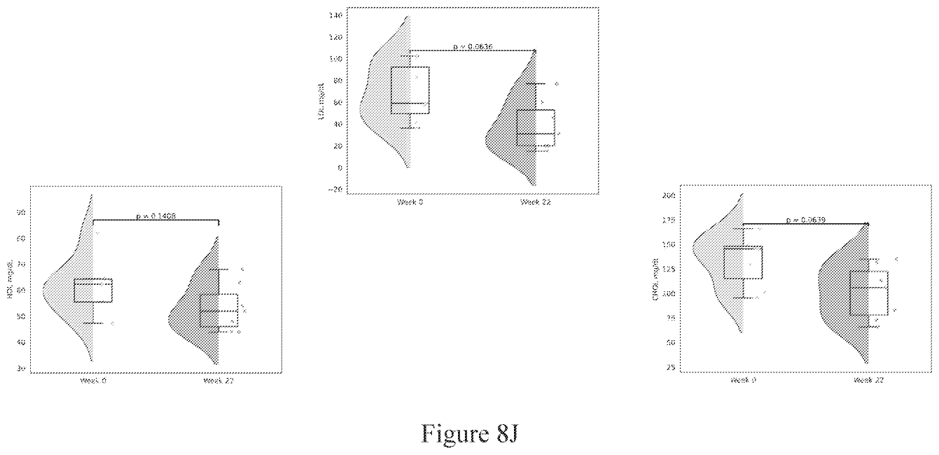

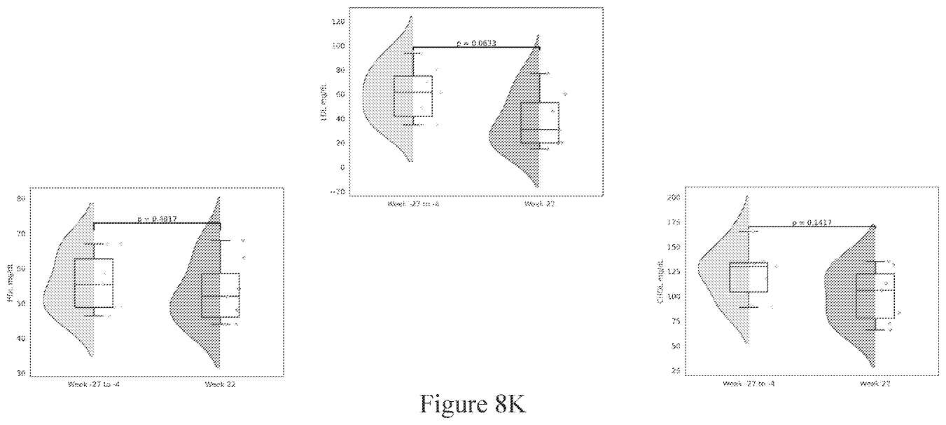

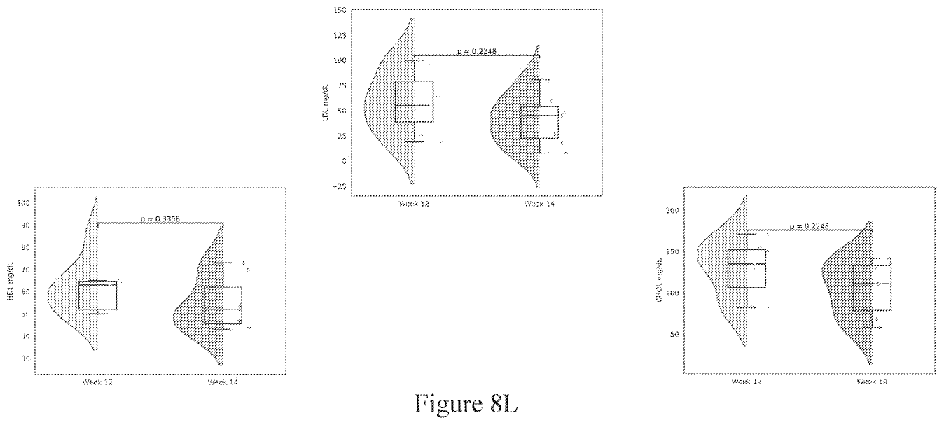

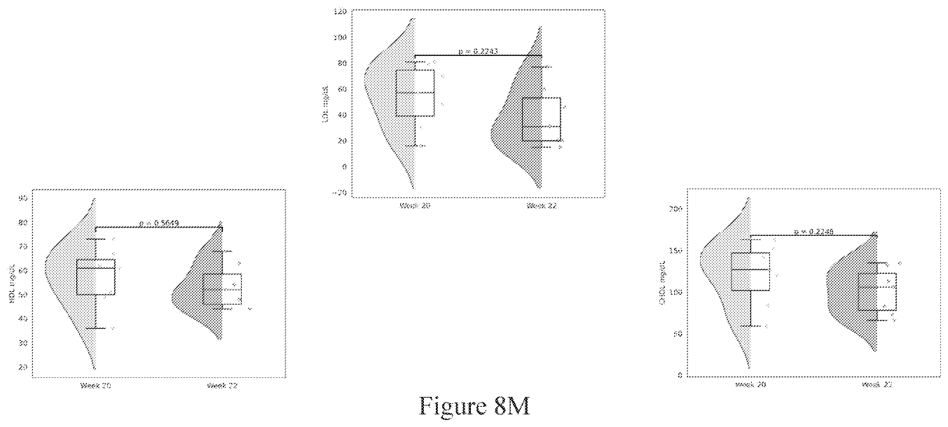

### Example 12—NHP Analysis of HIT01-K21Q-R218E (FIGS. 8A Through 8M)

This Example presents the results of a nonhuman primate (NHP) study concerning the immunization and antibody responses of HIT01-K21Q-R218E. The study protocol is schematically summarized in FIG. 8A. Briefly, one group of seven Cynomolgus monkeys was employed. The immunization scheme involved injecting 100 μg HIT01-K21Q-R218E into each animal with a carbomer-based adjuvant, ADJUPLEX at weeks 0, 4, 12, and 20. Serum was obtained from each animal at two-week intervals over 30 weeks.

Data from the study concerning the longitudinal serum antibody levels for each of the animals in the study are plotted in FIG. 8B, and a comparison between the anti-HIT01 or PCSK9 IgG response is depicted in FIG. 8C.

To assess efficacy, the LDL, cholesterol, and HDL levels from serum collected from each animal in the study were assessed for 30 weeks prior to and 30 weeks post injection. The data are presented in FIGS. 8D, 8E, and 8F.

Pooled data for LDL, HDL, and cholesterol serum levels measured for animals in the NHP study at pre-bleed t0 vs week 2 following the third immunization are presented in FIG. 8G. Similar data comparing pre-bleed t0 vs week 2 following the fourth immunization are presented in FIG. 8H. Similar data comparing pre-bleed t0 vs week 13 following the third immunization are presented in FIG. 8I. Similar data comparing pre-bleed t0 vs week 2 following the final immunization are presented in FIG. 8J. Similar data comparing the average levels pre-bleed vs week 2 following the fourth immunization are presented in FIG. 8K. Similar data comparing the LDL, HDL, and cholesterol serum levels measured at the third immunization vs week 2 following the third immunization are presented in FIG. 8L. Similar data comparing the LDL, HDL, and cholesterol serum levels measured at the fourth immunization vs week 2 following the fourth immunization are presented in FIG. 8M. The statistical analysis for these FIGS. 8G through 8M) was performed by a two-tailed Mann-Whitney U-Test with no continuity correction.

The results of this NHP study indicate that HIT01-K21Q-R218E immunization lowered LDL levels without disruption HDL levels. Significant differences were observed between (a) pre-bleed t0 vs week 2 post 3rd immunization for LDL, but not for HDL and Cholesterol (b) pre-bleed t0 vs week 2 post 4th immunization for LDL and Cholesterol, but not for HDL and (c) pre-bleed t0 vs all weeks post 3rd immunization for LDL, but not for HDL and Cholesterol. Differences which border on significant were observed between (a) week 0 vs week 2 post 4th immunization for LDL and Cholesterol, but not for HDL and (b) the average of all pre-bleed weeks vs week 2 post 4th immunization for LDL, but not HDL and cholesterol. No significant difference was observed between (a) the 3rd Immunization vs week 2 after for LDL, HDL, or Cholesterol or (b) the 4th Immunization vs week 2 after for LDL, HDL, or Cholesterol.

Thus, these results indicate that the impact of immunization in NHP with HIT01-K21Q-R218E is relatively short term in terms of reduced cholesterol, observed at 2 weeks after immunization but appearing to decline at 4 weeks. Moreover, individual differences between the separate animals in the study are noted, and these may complicate interpretation of the LDL changes.

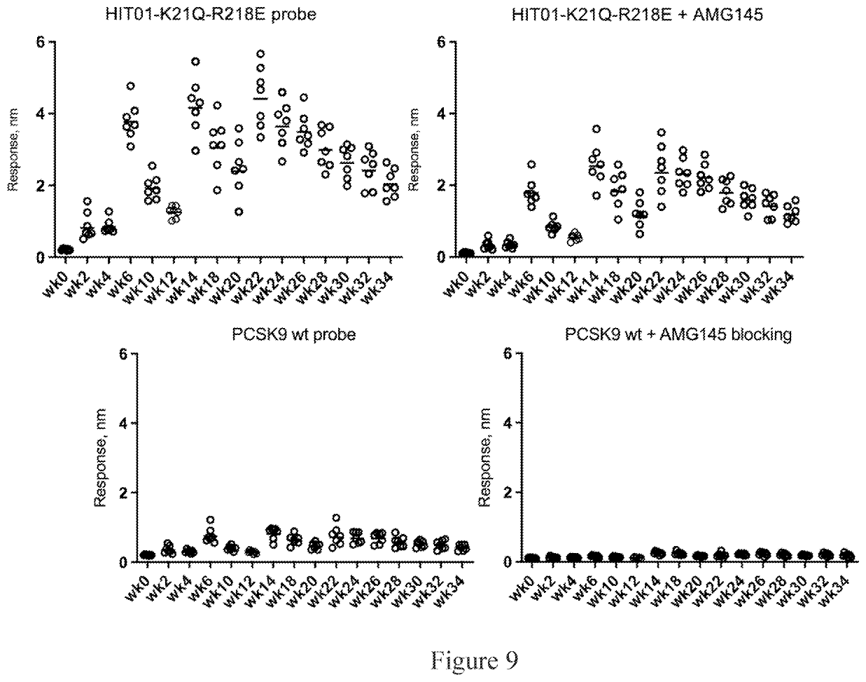

### Example 13—Competition Assay Using AMG145 (FIG. 9)

this Example presents the results of a study assessing the target specificity of HIT01-K21Q-R218E and wild type PCSK9.

Using sera from the NHP study (Example 11) through week 34 post injection, a competition assay was performed using antibody AMG145, which targets the LDL-R binding site. Immunogen-specific antibody response was assessed by immobilizing 50 μg/ml wt PCSK9 or HIT01-K21Q-R218E onto Ni-NTA sensors. The association with the indicated serum samples at 200-fold dilution was recorded and plotted out as depicted in FIG. 9. Similarly, for antibody blocking assays, wt PCSK9 or HIT01-K21Q-R218E loaded Ni-NTA sensors were loaded additionally with 50 μg/ml blocking antibody AMG145 for 300 s and assayed using serum samples from immunized animals. The results, plotted past 500 s, are depicted in FIG. 9. The data from this assay are presented in FIG. 9.

The results reveal a lower response when the serum is assayed using HIT01-K21Q-R218E blocked with AMG145 than when assayed using HIT01-K21Q-R218E alone. This result supports a conclusion that HIT01-K21Q-R218E elicits antibody responses targeting the LDL binding site. A comparison using wt PCSK9 alone or in conjunction with AMG145 blocking also was conducted. A reduction also is observed with AMG145 blocking in comparison to PCSK9 probe alone. The overall reduction is around 50% when the probes were block with AMG145, a LDL-R blocking antibody.

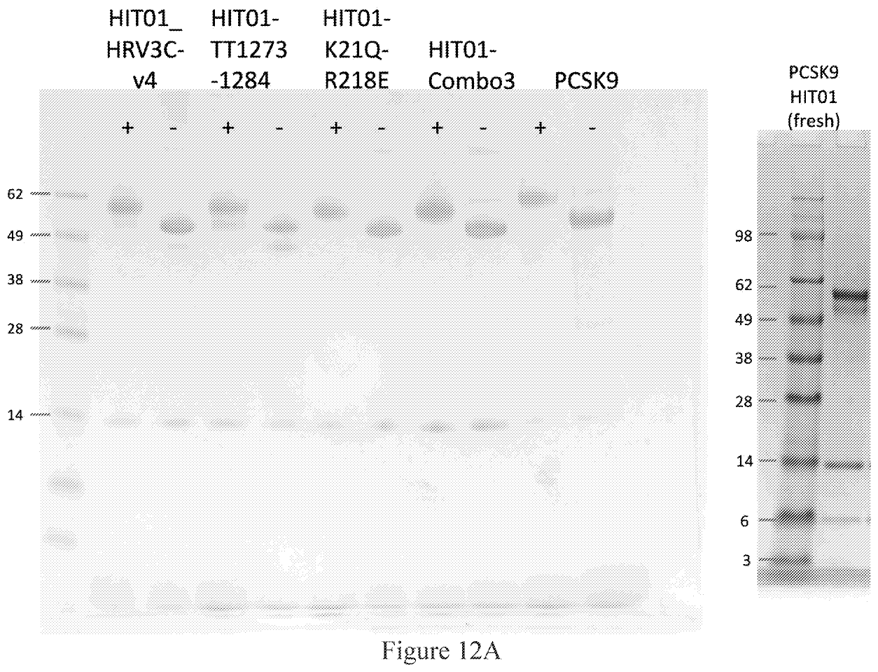

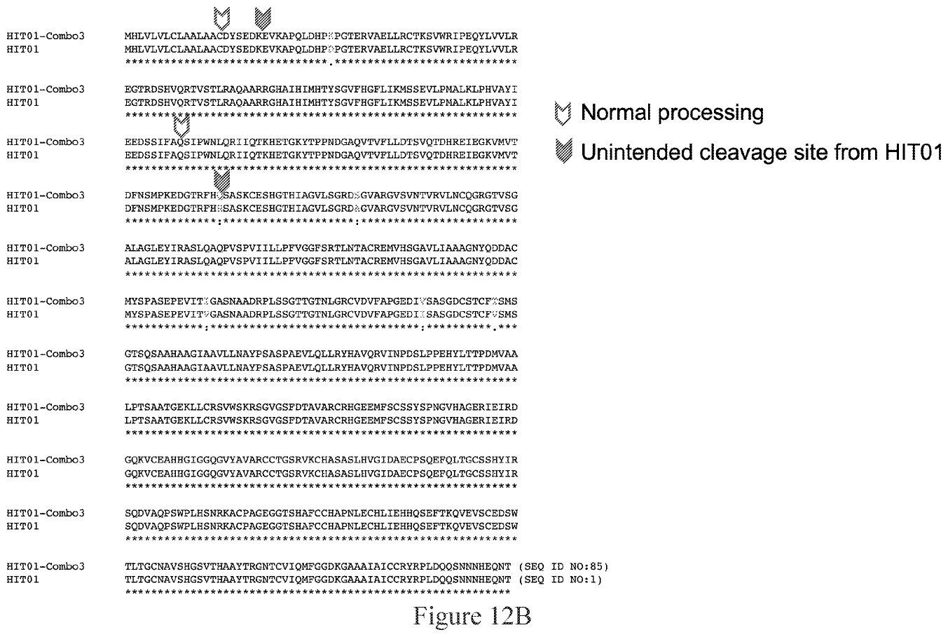

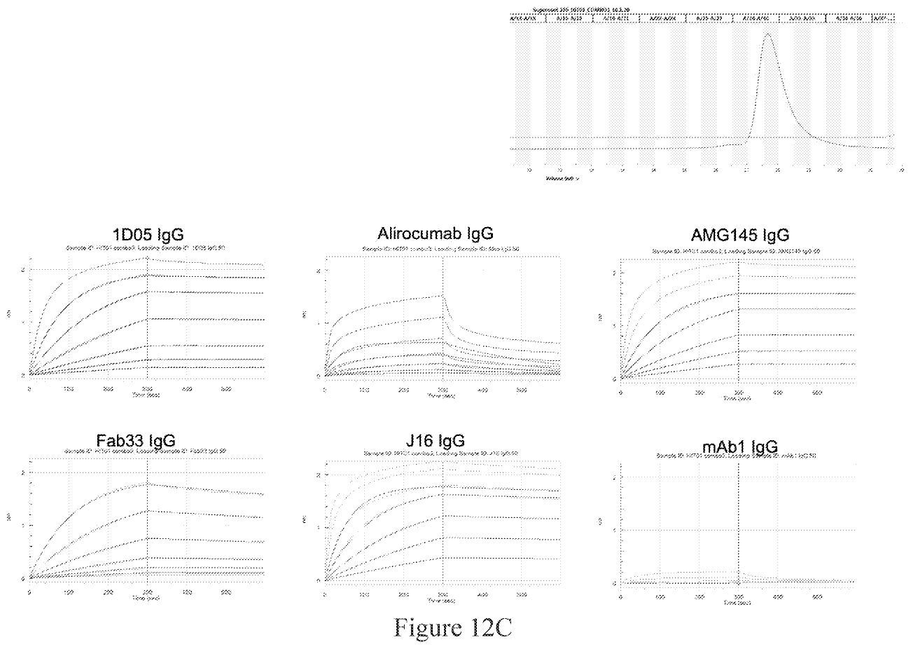

### Example 14—HIT01-Combo3 (FIGS. 12A Through 12C)

To evaluate HIT01 variants with no 8-mer identical to human proteins, such molecules were expressed in EXPI293 cell culture. One such protein is HIT01-Combo3 (SEQ ID NO:85).

After protein purification, the proteins were analyzed on SDS-PAGE gel (FIG. 12A). HIT01-Combo3 shows protein bands without degradation when compared to HIT01 on the right-hand side. Within this Figure, HIT01_HRV-3C-v4 (SEQ ID NO:5) represents the addition of HRV-3C to remove the C-terminal domain: HIT01-K21Q-R218E (SEQ ID NO:14) reflects mutations to reduce self-cleavage: HIT01-TT1273-1284 (SEQ ID NO:12) reflects the addition of T-cell help from tetanus toxoid at the C-terminal, and HIT01-Combo3 (SEQ ID NO:85, FIG. 12B) reflects mutations to remove 8-mers identical to the human sequence. FIG. 12B presents a sequence alignment comparing HIT01-Combo3 with HIT01, with an indication of normal processing and unintended cleavage of HIT01.

HIT01-Combo3 then was evaluated for its size exclusion chromatography profile and antibody binding kinetics (FIG. 12B). 50 μg/ml of antibodies were loaded on AHC sensors. The samples were the respective HIT01 variant 500 nm and 2× series dilutions. The SEC profile at the upper right corner of FIG. 12C reveals a homogenous protein preparation. HIT01-Combo3 binds to 5 our 6 PCSK9 monoclonal antibodies at the similar level to its parent HIT01 molecule (the binding curves at the bottom part of FIG. 12C).

These results demonstrated the engineering of a well-behaved HIT01 variant with no 8-mer identical to human proteins.

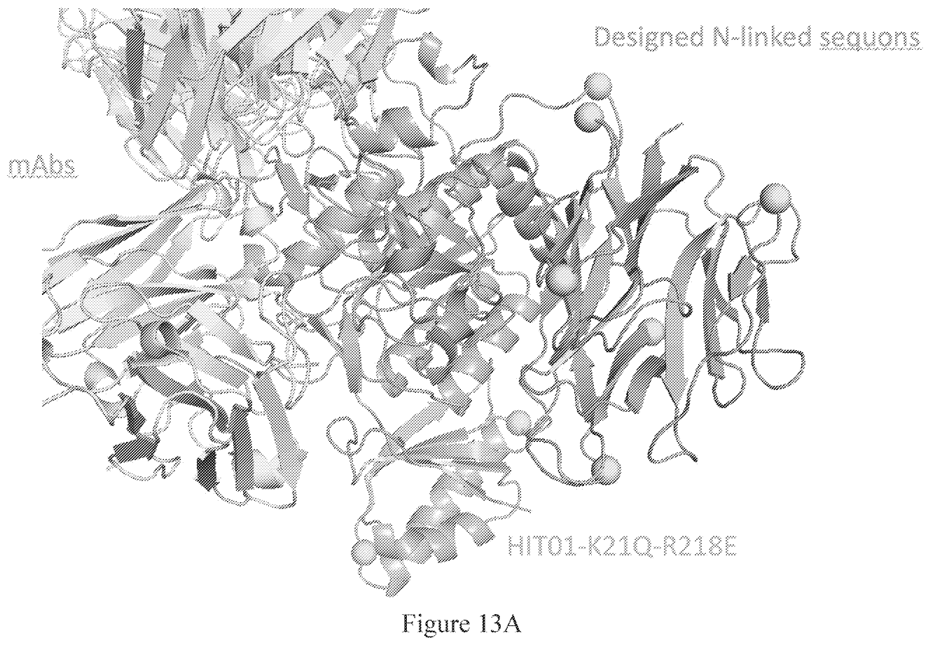

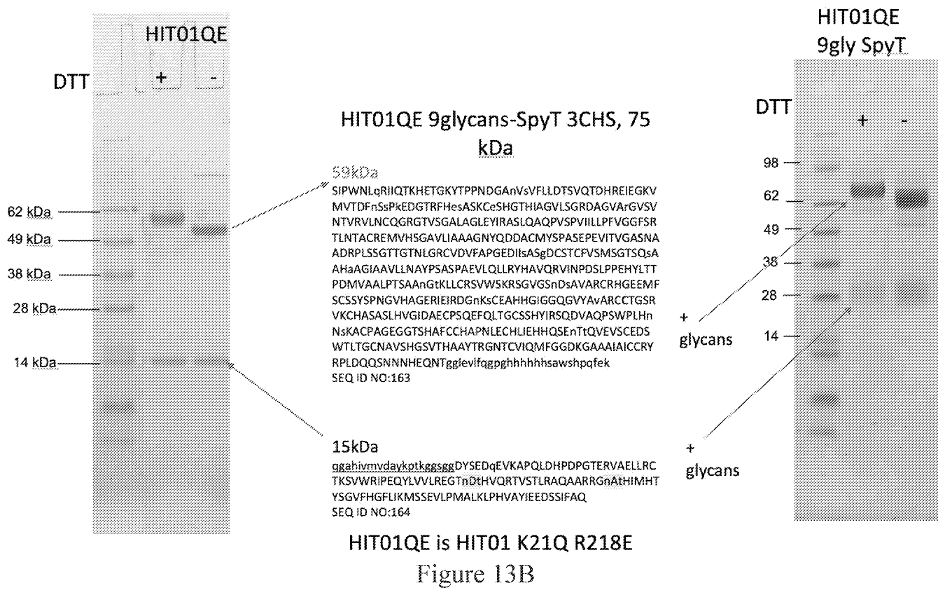

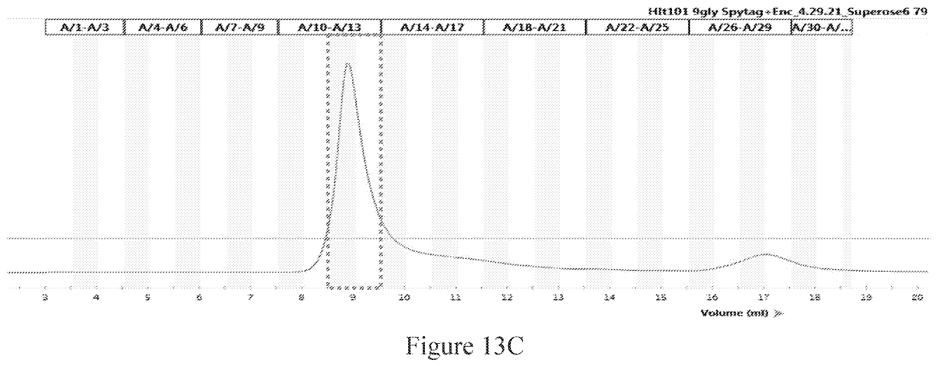

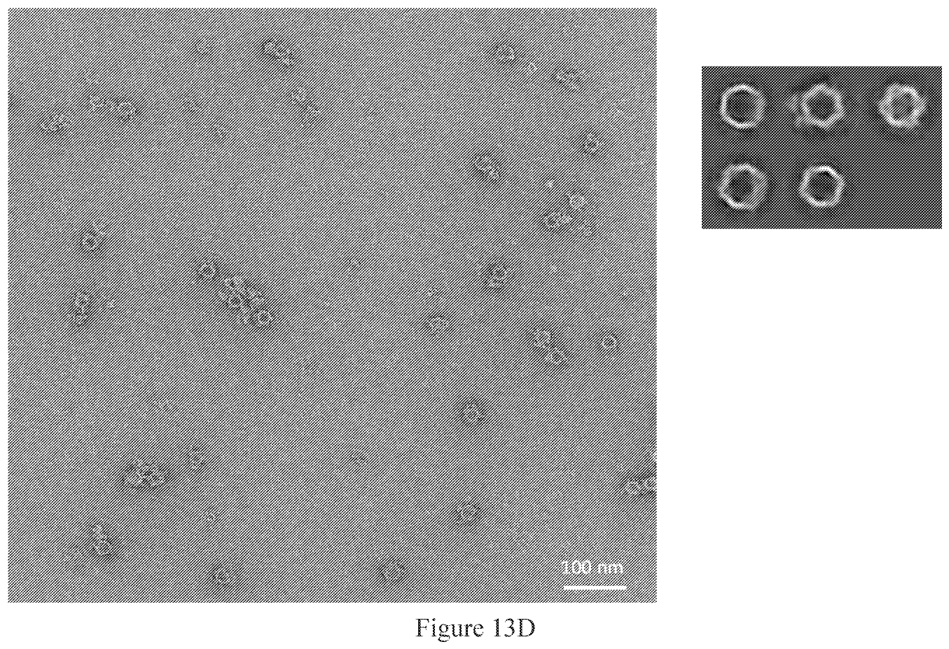

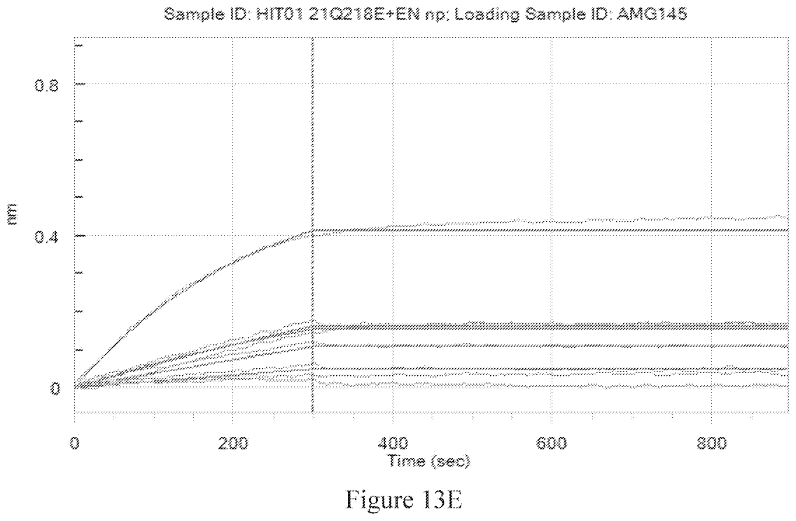

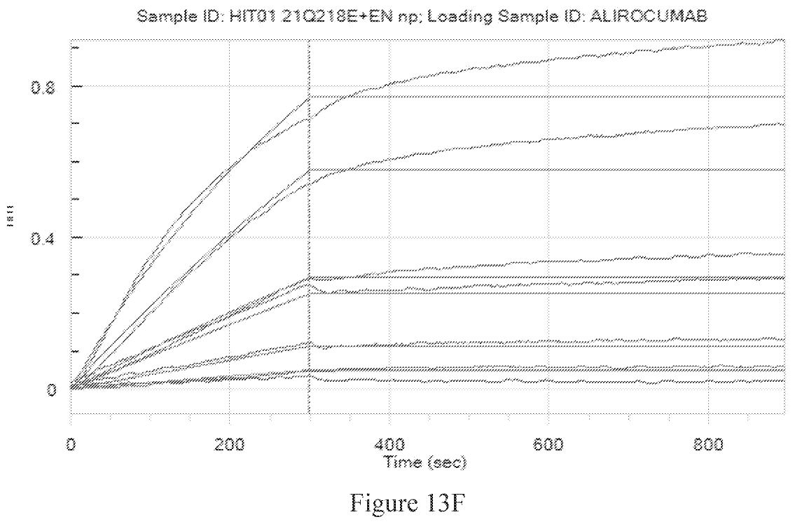

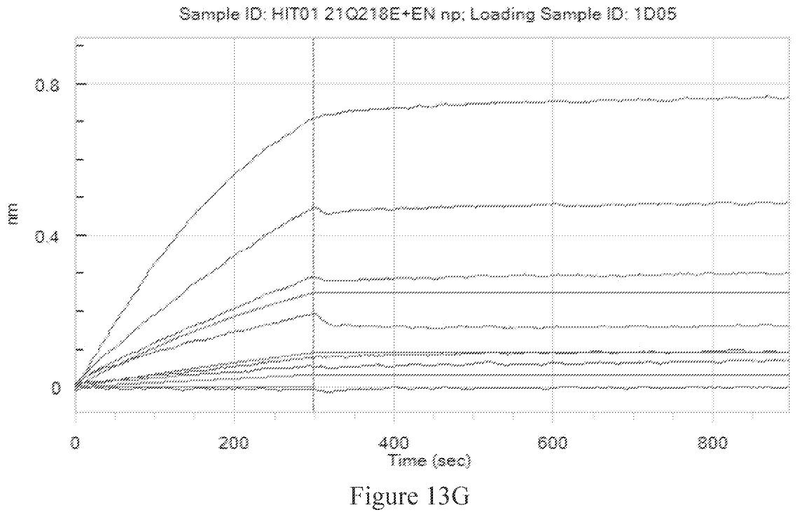

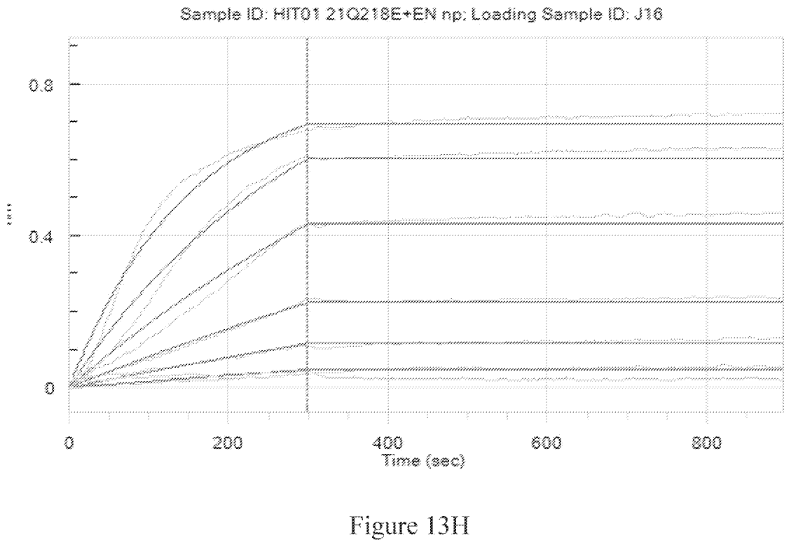

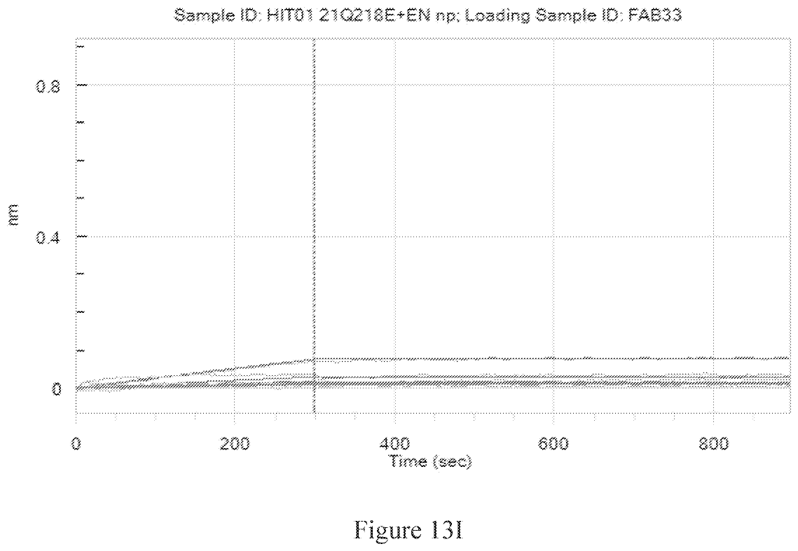

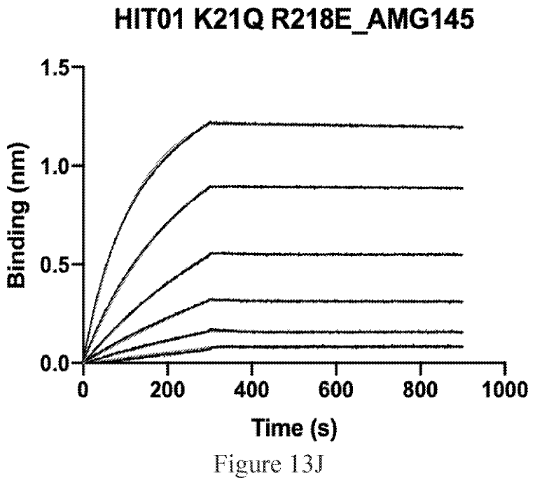

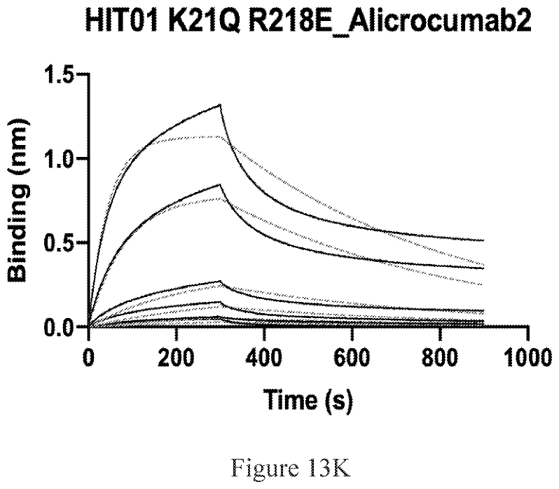

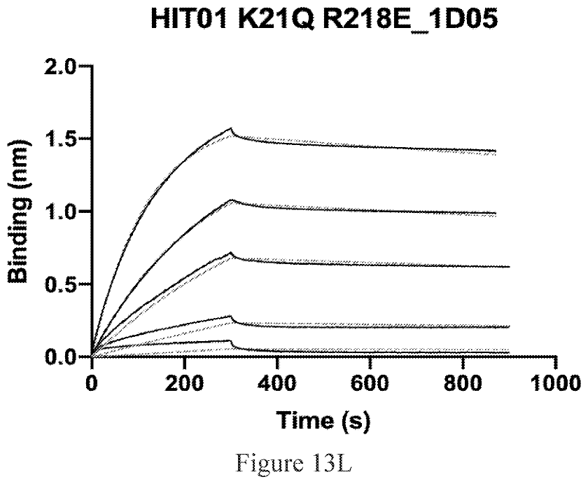

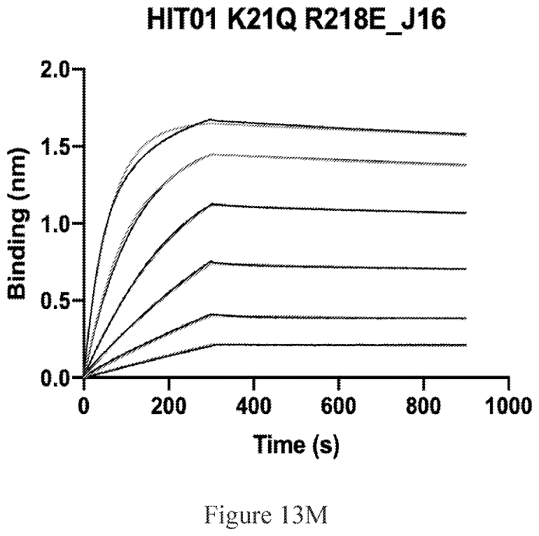

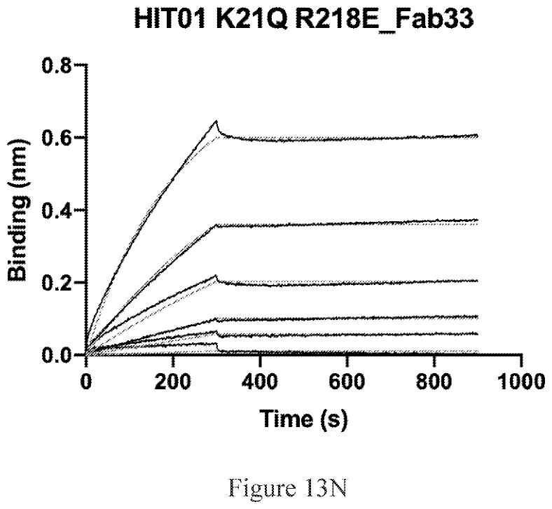

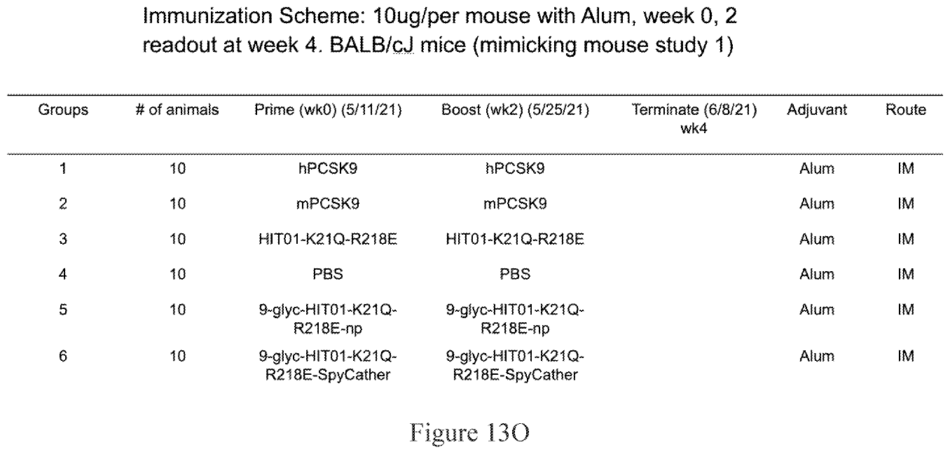

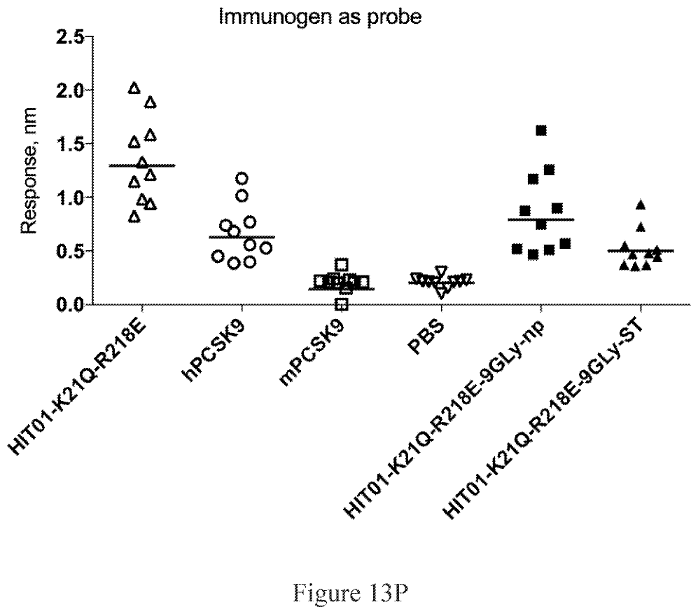

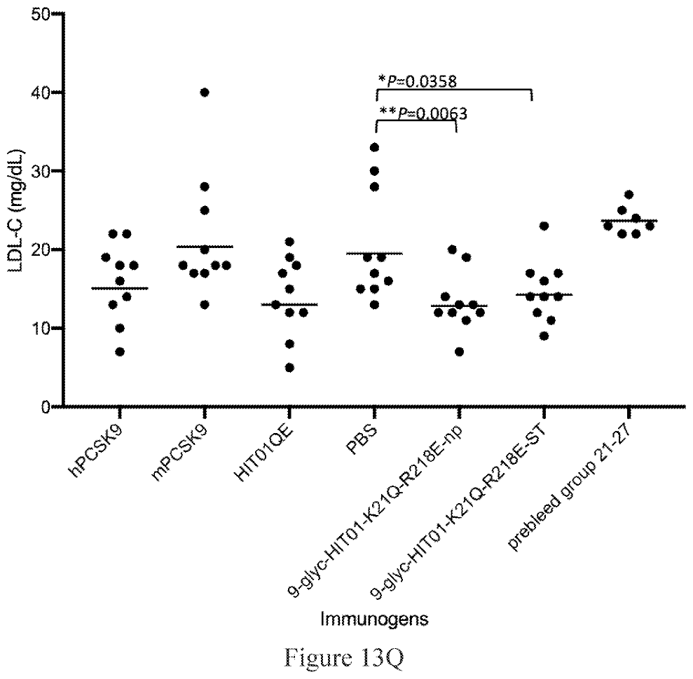

### Example 15—Glycocylation (FIGS. 13A Through 13Q)

This Example reports the engineering of PCDK9 mimics (HIT01 variants) on nanoparticles for improved antigenicity and stability.

FIG. 13A graphically illustrates glycan designs to enhance HIT01-K21Q-R218E nanoparticle solubility. A series of variants with mutations adding N-linked glycosylation sites to HIT01-K21Q-R218E molecules was designed and fabricated. SpyTag (SpyT) or Spy Catcher (SpyC) sequences also were added to the variants for conjugation with Spy Tag or Spy Cather self-assembling nanoparticles. The constructs are listed in Table 11 (SEQ ID Nos: 123-146).

These constructs were tested for their expression. One particular design, HIT01-K21Q-R218E 9glycans-SpyT (SEQ ID NO:146), behaved well in protein expression and nanoparticle conjugation. FIG. 13B presents the results of SDS-PAGE gel analysis for purified HIT01-K21Q-R218E 9glycans-SpyT protein. The gel image on right hand side of the slide indicated a clear protein band shift up when compared to its parent construct HIT01-K21Q-R218E on the left-hand side. This is interpreted to represent glycan addition to the protein.

Another component of the nanoparticle conjugation system is encapsulin- Spy Cather nanoparticle. HIT01-K21Q-R218E 9glycans-SpyT was expressed and purified separately (the encapsulin-Spy Catcher nanoparticle was published in PCT Application No.: PCT/US2019/052419). To make nanoparticle conjugate, HIT01-K21Q-R218E 9glycans-SpyT and encapsulin-Spy Cather were mixed together with 1:1 molar ratio and incubated overnight at 4° C. Assembled HIT01-K21Q-R218E 9glycans-encapsulin nanoparticles were separated from un-conjugated encapsulin-Spy Catcher and HIT01-K21Q-R218E 9glycans-SpyT by loading the mixture to SEC column. As shown in FIG. 13C, the peak at 9 ml elution volume is the conjugated product.

The formation of HIT01-K21Q-R218E 9glycans-encapsulin nanoparticles was further verified using negative stain electron microcopy. The image in FIG. 13D shows clearly the encapsulin core nanoparticle and the PCSK9 protein on the surface of nanoparticle core. Taken together, these results demonstrated the successful addition of glycan on HIT01-K21Q-R218E molecules. Moreover, HIT01-K21Q-R218E nanoparticle immunogens were successfully produced by using HIT01-K21Q-R218E glycan variants and Spy Tag nanoparticle system.

As shown in FIGS. 13E through 13N, HIT01-K21Q-R218E 9glycans-encapsulin nanoparticles immunogen was evaluated for its antibody binding activity. For the experiments, 10 μg/ml of antibodies were loaded on the AHC sensors. The samples consisted of the indicated HIT01 variant 500 nm and 2× series dilutions. For 4 out of 5 antibodies tested, HIT01-K21Q-R218E 9glycans-encapsulin bind tightly to the four antibodies without off-rate. These results demonstrated HIT01-K21Q-R218E 9glycans-encapsulin has higher affinity to a panel of monoclonal antibodies that block LDL-R receptor binding to PCSK9. This suggests the nanoparticle immunogen could elicit better antibody responses in animals than monomeric form of HIT01-K21Q-R218E molecule.

Animal studies in BALB/cJ mice also were performed using the HIT01-K21Q-R218E 9glycans-encapsulin nanoparticles immunogen. The immunization scheme is depicted in FIG. 13O and was similar to the immunization schemes discussed above in Examples 5 and 11. 10 μg/per mouse were injected intramuscularly with Alum, week 0, boosted at 2, with the readout at week 4.

One result of the study is depicted in FIG. 13P. The assay was conducted similarly as described in connection with FIG. 7B in Example 11. Briefly, terminal bleed sera from the mice in the study was diluted 200-fold in 1% BSA PBS. 50 μg/ml of the indicated immunogen was immobilized on a Ni-NTA sensor. For antibody blocking, 50 μg/ml was loaded onto the immunogen-loaded sensor. The results indicated that the best recognition of this sera was to HIT01-K21Q-R281E (first column on left). The reactivity of the HIT01-K21Q-R218E 9glycans-encapsulin nanoparticles and HIT01-K21Q-R218E 9glycans-encapsulin Spy Catcher immunogens, however, is shown to be higher than the PBS control and the mouse PCSK9 immunogen.

Another result of the study is depicted in FIG. 13Q, which depicts the LDL-C level in the mice treated with HIT01-K21Q-R218E 9glycans-encapsulin nanoparticles and HIT01-K21Q-R218E 9glycans-encapsulin Spy Tag immunogens, wild type human PCSK9, wild type mouse PCSK9, HIT01-K21Q-R218E, prebled group 21-27, as well as a PBS control. The analysis was conducted in accordance with a two-tailed Mann-Whitney test. The results in FIG. 13Q show that both HIT01-K21Q-R218E 9glycans-encapsulin nanoparticles and HIT01-K21Q-R218E 9glycans-encapsulin Spy Tag immunogens can reduce LDL-C slightly more than human PCSK9 (first column), and substantially better than mouse PCSK9 (second column) or PBS (fourth column) or the prebled group (seventh column) and were on par with HIT01-K21Q-R218E. Statistical significance relative to PBS is as shown in the figure.

### TABLES OF SEQUENCES

The following tables (Tables 1 through 11) present certain sequences of proteins/polypeptides referred to in this specification. With respect to the indicated sequences, certain of them contain C-terminal “tag” sequences, which are optional. In particular, for sequences containing a C-terminal sequence of “gglvpr/gshhhhhhsawshpqfek” (SEQ ID NO:159), which represents a thrombin cleavage site plus HIS tag and strep tag useful for purification, the protein/polypetide with the associated name can vary from the recited sequence by (a) lacking the entirety of SEQ ID NO: 159 or (b) lacking the portion of SEQ ID NO: 159 following the “/” character. Furthermore, for sequences containing a C-terminal sequence of “gglevlfqg/pghhhhhhsawshpqfek” (SEQ ID NO:160), which represents an HRVC3 cleavage site plus HIS tag and strep tag useful for purification, the protein/polypetide with the associated name can vary from the recited sequence by (a) lacking the entirety of SEQ ID NO: 160 or (b) lacking the portion of SEQ ID NO:160 following the “/” character. Furthermore, for sequences containing a C-terminal sequence of “ggggsggggsmknldcwvdneedidvilkkstilnldinndiisdisgfnssvitypdaqlvpgingkaihlvnnessevivhka mdieyndmfnnftvsfwlrvpkvsashleqygtneysiissmkkhslsigsgwsvslkgnnliwtlkdsagevrqitfrdlpdkf nay lankwwfititndrlssanlyingvlmgsaeitglgairednnitlkldrennnnqyvsidkfrifckalnpkeieklytsylsitfl rdfwgnplry dteyylipvassskdvqlknitdymyltnapsytngklniyyrrlynglkfiikrytpnneidsfvksgdfiklyvsy nnnehivgypkdgnafnnldrilrvgynapgiplykkmeavklrdlktysvqlklyddknaslglvgthngqigndpnrdilias nwyfnhlkdkilgcdwyfvptdegwtndGGGLVPQQSGAHIVMVDAYKPTKgsghhhhhhsawshpqfe k” (SEQ ID NO: 161), which represents rTTHC in lower case, then linker plus Spy Tag in upper case, followed by C-terminal purification tags, the protein/polypetide with the associated name can vary from the recited sequence by lacking the entirety of SEQ ID NO:161. Furthermore, for sequences containing a C-terminal sequence of “ggglvpqqsgAHIVMVDAYKPTKgsghhhhhhsawshpqfek” (SEQ ID NO: 162), which represents a thrombin cleavage site in lower case, then Spy Tag in upper case, then and purification tags in lower case, the protein/polypetide with the associated name can vary from the recited sequence by lacking the entirety of SEQ ID NO: 162.

Lengthy table referenced here
US20240299510A1-20240912-T00001
Please refer to the end of the specification for access instructions.

Lengthy table referenced here
US20240299510A1-20240912-T00002
Please refer to the end of the specification for access instructions.

Lengthy table referenced here
US20240299510A1-20240912-T00003
Please refer to the end of the specification for access instructions.

Lengthy table referenced here
US20240299510A1-20240912-T00004
Please refer to the end of the specification for access instructions.

Lengthy table referenced here
US20240299510A1-20240912-T00005
Please refer to the end of the specification for access instructions.

Lengthy table referenced here
US20240299510A1-20240912-T00006
Please refer to the end of the specification for access instructions.

Lengthy table referenced here
US20240299510A1-20240912-T00007
Please refer to the end of the specification for access instructions.

Lengthy table referenced here
US20240299510A1-20240912-T00008
Please refer to the end of the specification for access instructions.

Lengthy table referenced here
US20240299510A1-20240912-T00009
Please refer to the end of the specification for access instructions.

Lengthy table referenced here
US20240299510A1-20240912-T00010
Please refer to the end of the specification for access instructions.

Lengthy table referenced here
US20240299510A1-20240912-T00011
Please refer to the end of the specification for access instructions.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

LENGTHY TABLES
The patent application contains a lengthy table section. A copy of the table is available in electronic form from the USPTO web site (https://seqdata.uspto.gov/?pageRequest=docDetail&DocID=US20240299510A1). An electronic copy of the table will also be available from the USPTO upon request and payment of the fee set forth in 37 CFR 1.19(b)(3).

1. A composition comprising a polypeptide comprising a sequence of amino acids consisting essentially of SEQ ID NO: 145 or SEQ ID NO: 146.

2-26. (canceled)

27. The composition of claim 1, further comprising an immunogenic carrier.

28. The composition of claim 27, wherein the immunogenic carrier is selected from the group consisting of rTTHc, a nanoparticle, SPYTAG, and SPYCATCHER.

29-33. (canceled)

34. The composition of claim 28, wherein the nanoparticle contains a SPYTAG.

35. (canceled)

36. The composition of claim 28, wherein the nanoparticle contains a SPYCATCHER.

37-62. (canceled)

63. A method of preventing, alleviating, or treating a condition selected from atherosclerosis, coronary artery disease, cardiovascular disease, acute coronary syndrome, a dyslipidemia, and Alzheimer's disease in an individual, comprising administering a therapeutically effective amount of the composition of claim 1 to the individual.

64. The method of claim 63, wherein the composition is administered in combination with at least one additional agent selected from the group consisting of a statin, a bile acid sequestrant, niacin, a fibric acid derivative, and a long chain alpha, omega-dicarboxylic acid.

65-71. (canceled)

72. A nucleic acid encoding a polypeptide comprising a sequence of amino acids consisting essentially of SEQ ID NO: 145 or SEQ ID NO: 146.

73. The nucleic acid of claim 72, wherein the nucleic acid is an RNA molecule.

74. (canceled)

75. The nucleic acid of claim 72, operably linked to a promoter.

76. A genetic vector comprising the nucleic acid of claim 72.

77. The vector of claim 76, wherein the vector is a viral vector.

78. A host cell comprising the vector of claim 76.

79. An immunogenic composition comprising the vector of claim 76 and a carrier.

80. A method of preventing, alleviating, or treating a cancer in an individual, comprising administering a therapeutically effective amount of the composition of claim 1.

81. The method of claim 80, further comprising administering at least one cancer therapeutic agent to the individual.

82. The method of claim 81. wherein the cancer therapeutic agent is an immune checkpoint therapy agent.
